(19) United States
(12) Patent Application Publication
(10) Pub. No.: US 2010/0137294 A1
(43) Pub. Date: Jun. 3, 2010

(54) COMPOUNDS, COMPOSITIONS AND METHODS FOR CONTROLLING INVERTEBRATE PESTS

(75) Inventors: Robin Beat Gasser, Victoria (AU); Adam McCluskey, New South Wales (AU)

Correspondence Address: SHERIDAN ROSS PC, 1560 BROADWAY, SUITE 1200, DENVER, CO, 80202, US

(73) Assignees: GENETIC TECHNOLOGIES LIMITED, Victoria (AU); MEAT & LIVESTOCK AUTRALIA LIMITED, New South Wales (AU)

(21) Appl. No.: 12/514,877

(22) PCT Filed: Nov. 15, 2007
(86) PCT No.: PCT/AU2007/001762

§ 371 Date: Jan. 22, 2010

(30) Foreign Application Priority Data
Nov. 15, 2006 (AU) 2006906383
May 25, 2007 (AU) 2007902820

(51) Int. Cl. A01N 43/60; A01N 43/36; A01N 43/84; C07D 491/08; A01P 15/00

(52) U.S. Cl. 514/228.2; 514/411; 514/232.8; 514/254.02; 548/430; 544/61; 544/142; 544/372

(57) ABSTRACT

The present application discloses the Tv-stp-1 serine/threonine phosphatase gene from Trichostrongylus vitrinus, and compounds that are useful as invertebrate control agents.

### CROSS-REFERENCE TO RELATED APPLICATIONS

The present application claims priority from Australian Provisional Patent Application No 2006906383 filed on 15 Nov. 2006 and Australian Provisional Patent Application No 2007902820 filed on 25 May 2007, the contents of which are incorporated herein by reference.

### FIELD OF THE INVENTION

The present invention relates to invertebrate control agent(s) having pest controlling activity and to methods of controlling invertebrate pests by treatment with the aforementioned agents.

### BACKGROUND OF THE INVENTION

Invertebrates are animals that lack a vertebral column, or backbone. They include the protozoans, annelids, cnidarians, echinoderms, flatworms, nematodes, mollusks, and arthropods. In fact, more than 90% of living animals are invertebrates, with a worldwide distribution.

Amongst the different types of invertebrates, there are many which are considered pests and their control is highly desirable. For example, arthropods are a large phylum of animals and include the insects, arachnids, crustaceans, and others. Control of insects by the use of insecticides has been an integral part of agricultural practices for many years, and often represents the difference between profitable crop production for farmers and no marketable crop at all, and the value of insecticides in controlling human and animal diseases has been dramatic.

Another group of invertebrates which constitute a threat to animal husbandry are the parasitic helminths. For example, nematodes are roundworms with elongated, fusiform, or saclike bodies covered with cuticle, are virtually ubiquitous in nature, inhabiting soil, water and plants, and are, importantly, involved in a wide range of animal and plant parasitic diseases.

The roundworm parasites of mammals belong to the phylum Nematoda. The roundworms include the hookworms (e.g. Necator americanus and Ancylostoma duodenale), common roundworms (e.g. the common roundworm Ascaris lumbricoides), whipworms (e.g. Trichuris trichiura), and pinworms (e.g. Enterobius vermicularus), Strongyloides stercoralis, Trichinella species and filarial worms, such as Wuchereria bancrofti. Other important roundworm parasites include Ancylostoma caninum (of dogs and humans), Strongylus vulgaris (infections of horses), Trichostrongylus colubriformis, Teladorsagia circumcincta (infections of sheep and goats), Haemonchus contortus (infections of sheep and goats), Ostertagia ostertagi, Haemonchus placei (infections of cattle), Ascaris suum (infections of pigs), Toxascaris leonina or Uncinaria stenocephala (infections of dogs), Toxocara species (infections of dogs, cats, humans and other) and Dirofilaria immitis (infections of cats and dogs).

Even subclinical parasitic infections are harmful to the host animal for a number of reasons; e.g. they deprive the host of food, injure organs or obstruct ducts, may elaborate substances toxic to the host, and provide a port of entry for other organisms. In other cases, the host may be a species raised for food and the parasite may be transmitted upon eating to infect the ingesting animal. It is highly desirable to eliminate such parasites as soon as they have been discovered.

More commonly, such infections are not asymptomatic. Helminth infections of mammals, particularly due to parasitic nematodes, are a source of great economic loss, especially of livestock and pets, e.g., sheep, cattle, horses, pigs, goats, dogs, cats, and birds, especially poultry. These animals must be regularly treated with anthelmintic chemicals in order to keep such infections under control, or else the disease may result in anaemia, diarrhoea, dehydration, loss of appetite, and/or even death.

The main currently available means of controlling helminth infections is with the use of anthelmintic chemicals, but these are only effective against resident worms present at the time of treatment. Therefore, treatment must be regular, since the animals are constantly exposed to infection; e.g. anthelmintic treatment with an avermectin is required monthly for most of the year to control Dirofilaria immitis or the dog heartworm. This is an expensive and labour intensive procedure. Due to the widespread use of anthelmintic chemicals, the worms develop resistance and so new classes of chemicals must be developed.

Therefore, it is highly desirable that new invertebrate pest control agents are developed.

### SUMMARY OF THE INVENTION

The present invention provides compound(s) for controlling invertebrate pest, compositions containing these compound(s), and methods for using same.

The present invention provides a method of controlling an invertebrate pest, comprising contacting said pest with at least one invertebrate control agent(s) having the Formula (I):

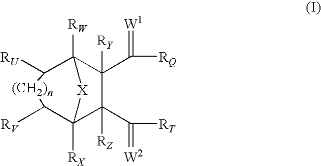

wherein:

n is 0, 1 or 2;

R_{Q} and —R_{T} are independently selected from —OR⁴ and —YA;

or R_{Q} and R_{T} together form a

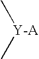

group;

A is independently selected from hydrogen, a substituted or unsubstituted aliphatic, alicyclic or aryl group, in which the carbon chain is optionally interrupted by one or more heteroatom(s), or is absent;

X is independently selected from O, S, CR¹R² or NR³;

Y is independently selected from N, NH, O or S;

each of W¹ and W² is independently selected from O, S, NR³ or CR¹R²;

each of R_{U} and R_{V} is independently selected from hydrogen, NR⁵R⁶, OR⁷, a substituted or unsubstituted aliphatic, alicyclic or aryl group, in which the carbon chain is optionally interrupted by one or more heteroatom(s), or together form an epoxide ring,

each of R¹, R², R³, R⁴, R⁵, R⁶, R⁷, R_{W}, R_{X}, R_{Y} and R_{Z} is independently selected from hydrogen or a substituted or unsubstituted aliphatic group in which the carbon chain is optionally interrupted by one or more heteroatom(s), or a salt or solvate thereof.

All invertebrate control agent(s) described herein may be used in pure form or in the form of a salt or solvate thereof of the agent. In a preferred embodiment, the salts or solvate include pharmaceutically and/or veterinarily acceptable salts or solvates thereof of the agent(s).

In another aspect, the present invention provides a method of treating and/or preventing a disease in a subject caused by an invertebrate pest comprising administering to the subject at least one invertebrate control agent(s) having the Formula (I):

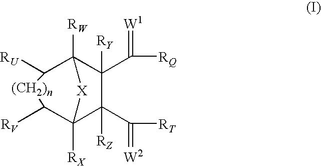

wherein n, X, R_{U}, R_{V}, R_{W}, R_{X}, R_{Y}, R_{Z}, W¹, W², R_{Q} and R_{T} have the same meaning as above, or a salt or solvate thereof.

In yet a further aspect, the present invention provides a use of a compound having the Formula (I):

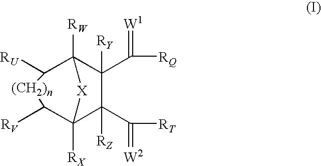

wherein n, X, R_{U}, R_{V}, R_{W}, R_{X}, R_{Y}, R_{Z}, W¹, W², R_{Q} and R_{T} are as specified above, or a salt or solvates thereof, for the manufacture of a medicament for treating and/preventing a disease in a subject caused by an invertebrate.

In another aspect, the present invention provides a compound according to Formula (I):

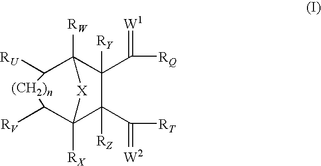

wherein:

n is 0, 1 or 2;

R_{Q} and —R_{T} are independently selected from —OR⁴ and —YA;

or R_{Q} and R_{T} together form a

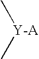

group;

A is independently selected from hydrogen, a substituted or unsubstituted aliphatic, alicyclic or aryl group, in which the carbon chain is optionally interrupted by one or more heteroatom(s), or is absent;

X is independently selected from O, S, CR¹R² or NR³;

Y is independently selected from N, NH, O or S;

each of W¹ and W² is independently selected from O, S, NR³ or CR¹R²;

each of R_{U} and R_{V} is independently selected from hydrogen, NR⁵R⁶, OR⁷, a substituted or unsubstituted aliphatic, alicyclic or aryl group, in which the carbon chain is optionally interrupted by one or more heteroatom(s), or together form an epoxide ring,

each of R¹, R², R³, R⁴, R⁵, R⁶, R⁷, R_{W}, R_{X}, R_{Y}, and R_{Z} is independently selected from hydrogen or a substituted or unsubstituted aliphatic group in which the carbon chain is optionally interrupted by one or more heteroatom(s);

or a salt or solvate thereof;

with the proviso that the compound is not selected from the group comprising:
N-(2-bromoethyl)-7-Oxabicyclo(2.2.1)heptane-2,3-dicarboximide;
N-(2-hydroxypropyl)-exo-(Z)-7-Oxabicyclo(2.2.1)heptane-2,3-dicarboximide;
N-phenyl-exo-(Z)-7-Oxabicyclo(2.2.1)heptane-2,3-dicarboximide;
N-exo-(Z)-7-Oxabicyclo(2.2.1)heptane-2,3-dicarboximide;
N-(o-methylphenyl)-exo-(Z)-7-Oxabicyclo(2.2.1)heptane-2,3-dicarboximide;
N-(p-chlorophenyl)-exo-(Z)-7-Oxabicyclo(2.2.1)heptane-2,3-dicarboximide;
N-(p-fluorophenyl)-exo-(Z)-7-Oxabicyclo(2.2.1) heptane-2,3 dicarboximide;
N-(o-chlorobenzyl)-exo-(Z)-7-Oxabicyclo(2.2.1)heptane-2,3-dicarboximide;
exo-7-oxabicyclo(2.2.1)heptane-2,3-dicarboximide;
N-(o-chlorobenzyl)-exo-(Z)-7-Oxabicyclo(2.2.1)heptane-2,3-dicarboximide;
N-(o-methoxyphenyl)-exo-(Z)-7-Oxabicyclo(2.2.1)heptane-2,3-dicarboximide;
N-(m-fluorophenyl)-exo-(Z)-7-Oxabicyclo(2.2.1)heptane-2,3-dicarboximide;
N-(m-ethoxyphenyl)-exo-(Z)-7-Oxabicyclo(2.2.1)heptane-2,3-dicarboximide;
N-(m-chloro-o-methylphenyl)-exo-(Z)-7-Oxabicyclo(2.2.1)heptane-2,3-dicarboximide;
N-(hydroxymethyl-acetate)-7-Oxabicyclo(2.2.1)heptane-2,3-dicarboximide;
N-(o-fluorophenyl)-exo-(Z)-7-Oxabicyclo(2.2.1)heptane-2,3-dicarboximide;
N-(2,4-dichlorophenyl)-exo-(Z)-7-Oxabicyclo(2.2.1)heptane-2,3-dicarboximide;
2,3-dimethyl-7-Oxabicyclo(2.2.1)heptane-2,3-dicarboxylic anhydride;
(3a-α,4-β,7-β,7a-α)-Hexahydro-3a-methyl-4,7-epoxyisobenzofuran-1,3-dione;
2-methyl-7-Oxabicyclo(2.2.1)heptane-2,3-dicarboxylic anhydride (palasonin);
hexahydro-3a,7a-dimethyl- (3a-α,4-β,7-β,7a-α) -4,7-Epoxybenzo (c) thiophene-1,3-dione;
hexahydro-3a-methyl-, (3a-α,4-β,7a-α)-4,7-Epoxybenzo(c)thiophene-1,3-dione;
N-[2-hydroxy-3-{4-(2-pyridyl)piperazin-1-yl}propyl]endo-7-oxabicyclo[2,2,1]heptane-2,3-dicarboximide;
N-[2-n-caproyloxy-3-{4-(2-pyridyl)piperazin-1-yl}propyl]endo-7-oxabicyclo[2,2,1]heptane-2,3-dicarboximide;
N-[2-cyclohexylcarbonyloxy-3-{4-(2-pyridyl)piperazin-1-yl}propyl]endo-7-oxa bicyclo[2,2,1]heptane-2,3-dicarboximide;
N-[2-n-hexadecanoyloxy-3-{4-(2-pyridyl)piperazin-1-yl}propyl]endo-7-oxabicyclo[2,2,1]heptane-2,3-dicarboxylmide;
N-[2-hexadecanoyloxy-3-[4-(3-trifluoromethylphenyl)piperazin-1-yl]propyl]endo-7-oxabicyclo[2,2,1]heptane-2,3-dicarboximide;
3-(N-sec-butylcarbamoyl)-7-Oxabicyclo(2.2.1)heptane-2-carboxylic acid;
3-(pentylcarbamoyl)-7-Oxabicyclo(2.2.1)heptane-2-carboxylic acid;
3-(dodecylcarbamoyl)-7-Oxabicyclo(2.2.1)heptane-2-carboxylic acid;
3-((4-chlorophenyl)amino)carbonyl)-(exo,exo)-7-Oxabicyclo(2.2.1)heptane-2-carboxylic acid;
monoethyl ester-(exo,exo)-7-Oxabicyclo(2.2.1)heptane-2,3-dicarboxylic acid;
3-carbamoyl-7-Oxabicyclo(2.2.1) heptane-2-carboxylic acid, ammonia salt;
7-Oxabicyclo(2.2.1)heptane-2,3-dicarboxylic acid;
(endo,exo)-7-Oxabicyclo(2.2.1)heptane-2,3-dicarboxylic acid;
(endo,endo)-7-Oxabicyclo(2.2.1)heptane-2,3-dicarboxylic acid;
monomethyl ester-7-Oxabicyclo(2.2.1) heptane-2,3-dicarboxylic acid;
isopropyl ester, exo-(Z)-7-Oxabicyclo(2.2.1)heptane-2,3-dicarboxylic acid;
   and
diethyl ester, (exo,exo)-7-Oxabicyclo(2.2.1)heptane-2,3-dicarboxylic acid.

Preferably, n is 0.

Preferably, X is selected from O, S or NR³. In an embodiment, X is O. In another embodiment, X is S. In a further embodiment, X is NR³. Preferably, R³ is selected from hydrogen or a substituted or unsubstituted aliphatic group. In an embodiment, R³ is H.

Preferably, Y is N. Preferably both W¹ and W² are O.

In an embodiment, A is an aliphatic group selected from a substituted or unsubstituted alkyl or alkene, in which the carbon chain is optionally interrupted by one or more heteroatom(s). Preferably, the aliphatic group is a substituted or unsubstituted alkyl group. In one embodiment, the alkyl group is a C₁₋₂₀ alkyl. Preferably, the alkyl group is a C₃₋₁₀ alkyl. Examples of such C₃₋₁₀ alkyl include, but are not limited to, —CH₂CH₂OH, —CH₂CH(OCH₃)CH₂OH, —CH₂CH₂CH₃, —CH₂(CH₂)₂CH₃, —CH₂(CH₂)₄CH₃, —CH₂(CH₂)₆CH₃ or —CH₂(CH₂)₈CH₃ groups.

In another embodiment, the alkyl group is a substituted alkyl group. In one preferred form, the alkyl is substituted with an alicyclic group. Examples of such alicyclic groups, but are not limited to,

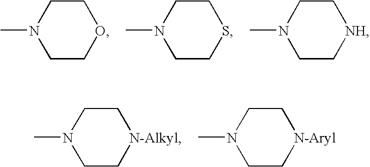

group.

In another embodiment, the alkyl group is substituted with a carboxylic acid group. Examples of such substituted alkyls include, but are not limited to, —(CH₂)₂CO₂H, —(CH₂)₃CO₂H, —(CH₂)₅CO₂H, or —(CH₂)₇CO₂H.

In yet another embodiment, the alkyl group is substituted with aryl ring. Examples of such alkylaryl groups include, but are not limited to,

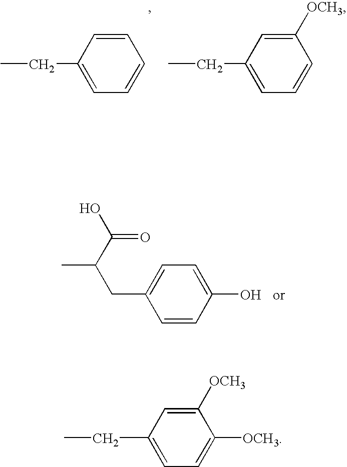

In yet another embodiment, the aliphatic group is an alkene, in which the carbon chain is optionally interrupted by one or more heteroatom(s). Preferably, the alkene is a C₃₋₂₀ alkene with one or more unsaturations. Preferably, the alkene has one unsaturation. Examples of such alkenes include, but are not limited to, —CH₂CH═CH₂; —CH₂CH₂CH═CH₂, —CH₂(CH₂)₃CH═CH₂ or —CH₂(CH₂)₅CH═CH₂.

In another embodiment, A is a substituted or unsubstituted alicyclic group. In one embodiment, the alicyclic group is a heterocyclic ring. Preferably, the heterocyclic ring has 3 to 20 ring members. More preferably, 6 ring members.

In yet a further embodiment, A is a substituted or unsubstituted aryl group, in which the carbon chain is optionally interrupted by one or more heteroatom(s). In one embodiment, the aryl group is a substituted or unsubstituted phenyl, biphenyl, naphthyl, indanyl, or indenyl. Preferably, the aryl group is a phenyl. More preferably, the phenyl is a substituted phenyl. Even more preferably, the substituted phenyl is a hydroxylmethyl phenyl. In another embodiment, the aryl group is a heteroaryl group.

In a further embodiment, the invertebrate control agent(s) has a structure according to Formula (II):

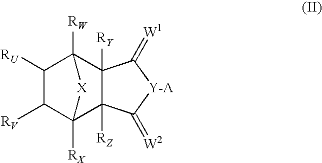

wherein A, Y, X, W¹, W², R_{U}, R_{V}, R_{W}, R_{X}, R_{Y} and R_{Z} are as specified above,

or a salt or solvate thereof.

In another embodiment, the invertebrate control agent(s) has a structure according to Formula (III):

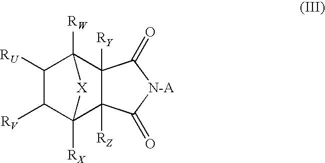

wherein A, X, R_{U}, R_{V}, R_{W}, R_{X}, R_{Y} and R_{Z} are as specified above,

or a salt or solvate thereof.

In a further embodiment, the invertebrate control agent(s) has a structure according to Formula (IV):

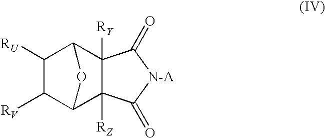

wherein A, R_{U}, R_{V}, R_{Y} and R_{Z} are as specified above,

or a salt or solvate thereof.

In a yet a further embodiment, the invertebrate control agent(s) has a structure according to Formula (V):

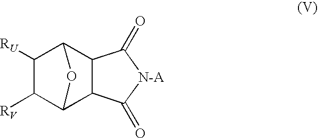

wherein A, R_{U} and R_{V} are as specified above,

or a salt or solvate thereof.

In a further embodiment, the invertebrate control agent(s) has a structure according to Formula (VI):

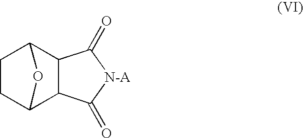

wherein A is as specified above,

or a salt or solvate thereof.

In a further embodiment, the invertebrate control agent(s) has a structure according to Formula (VII):

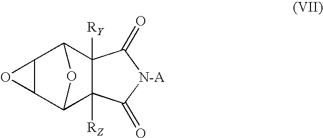

wherein A, R_{Y} and R_{Z} are as specified above,

or a salt or solvate thereof.

In yet another embodiment, the invertebrate control agent(s) has a structure according to Formula (VIII):

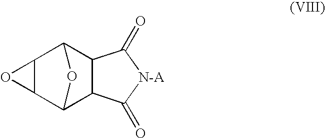

wherein A is as specified above,

or a salt or solvate thereof.

In another embodiment, the invertebrate control agent(s) has a structure according to Formula (IX):

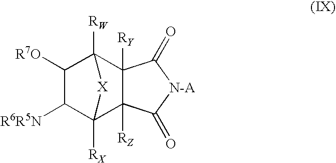

wherein A, X, R_{W}, R_{X}, R_{Y}, R_{Z}, R⁵, R⁶ and R⁷ are as specified above,

or a salt or solvate thereof.

Preferably, R⁷ is H.

In another embodiment, the invertebrate control agent(s) has a structure according to Formula (X):

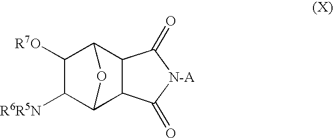

wherein A, R⁵, R⁶ and R⁷ are as specified above,

or a salt or solvate thereof.

In yet another embodiment, the invertebrate control agent(s) has a structure according to Formula (XI):

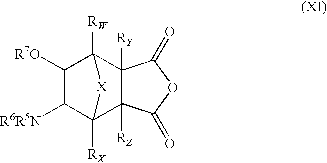

wherein X, R_{W}, R_{X}, R_{Y}, R_{Z}, R⁵, R⁶ and R⁷ are as specified above,

or a salt or solvate thereof.

In yet another embodiment, the invertebrate control agent(s) has a structure according to Formula (XII):

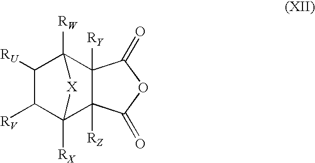

wherein X, R_{U}, R_{V}, R_{W}, R_{X}, R_{Y} and R_{Z} are as specified above,

or a salt or solvate thereof.

In yet a further embodiment, the invertebrate control agent(s) has a structure according to Formula (XIII):

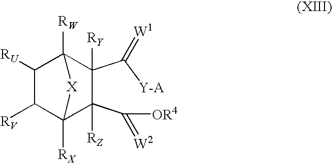

wherein X, Y, A, R_{U}, R_{V}, R_{W}, R_{X}, R_{Y}, R_{Z}, W¹, W² and R⁴ are as specified above,

or a salt or solvate thereof.

In yet a further embodiment, the invertebrate control agent(s) has a structure according to Formula (XIV):

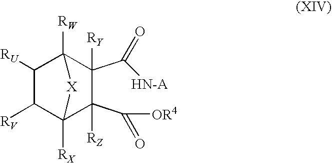

wherein A, X, R_{U}, R_{V}, R_{W}, R_{X}, R_{Y}, R_{Z} and R⁴ are as specified above,

or a salt or solvate thereof.

In an embodiment, the invertebrate control agent(s) has a structure according to Formula (XV):

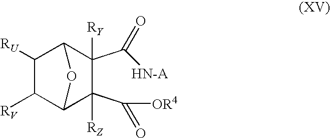

wherein A, R_{U}, R_{V}, R_{Y}, R_{Z} and R⁴ are as specified above,

or a salt or solvate thereof.

In yet another embodiment, the invertebrate control agent(s) has a structure according to Formula (XVI):

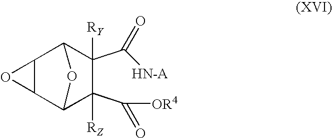

wherein A, R_{Y}, R_{Z} and R⁴ are as specified above,

or a salt or solvate thereof.

In a further embodiment, the invertebrate control agent(s) has a structure according to Formula (XVII):

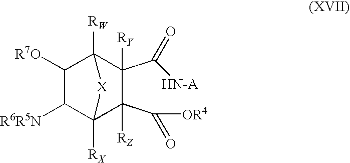

wherein A, X, R_{W}, R_{X}, R_{Y}, R_{Z}, R⁴, R⁵, R⁶ and R⁷ are as specified above;

or a salt or solvate thereof.

In another embodiment, the invertebrate control agent(s) has a structure according to Formula (XVIII):

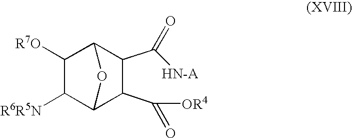

wherein A, R⁴, R⁵, R⁶ and R⁷ are as specified above;

or a salt or solvate thereof.

Preferably, the invertebrate pest is selected from the group comprising: arthropods and helminths. More preferably, the invertebrate pest is a helminth. In particularly preferred form, the helminth is selected from nematodes, cestodes or trematodes. Examples of such nematodes include, but are not limited to, Trichostrongylus sp or Haemonchus sp.

In an embodiment, the disease is parasitosis.

In one embodiment, the subject is an animal. Examples of such animals include, but are not limited to, humans, non-human simian, bovine, porcine, equine, ovine, canine, feline, avian, murine and caprine species.

Preferably, compounds of the present invention may be selected from compounds, or their salt or solvates thereof, but are not limited, to the following:

TABLE 1
Non-Limiting Examples of compounds of the present invention and/or
compounds useful in the practice of the methods of present invention.

| Name | Structure |
|---|---|
| N-propyl-7-Oxabicyclo(2.2.1)heptane-2,3-dicarboximide (4) | 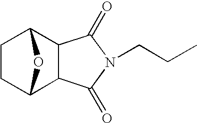 |
| N-butyl-7-Oxabicyclo(2.2.1)heptane-2,3-dicarboximide (5) | 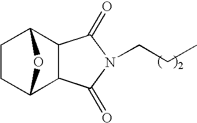 |
| N-octyl-7-Oxabicyclo(2.2.1)heptane-2,3-dicarboximide (6) | 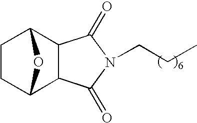 |
| N-decyl-7-Oxabicyclo(2.2.1)heptane-2,3-dicarboximide (7) | 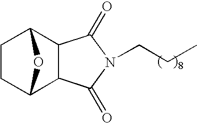 |
| N-(3-butenyl)-7-Oxabicyclo(2.2.1)heptane-2,3- dicarboximide (8) 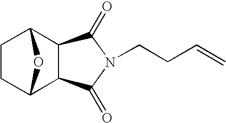 | 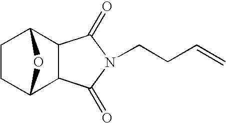 |
| 3-(butylcarbamoyl)-7-Oxabicyclo(2.2.1)heptane-2- carboxylic acid (9) | 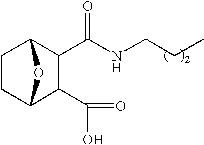 |
| 3-(decylcarbamoyl)-7-Oxabicyclo(2.2.1)heptane-2- carboxylic acid (10) | 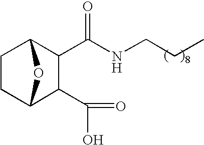 |
| 3-(4-benzylcarbamoyl)-7-Oxabicyclo(2.2.1)heptane-2- carboxylic acid (11) | 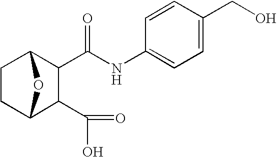 |
| 3-(Ethylmorpholine carbamoyl)-7- Oxabicyclo(2.2.1)heptane-2-carboxylic acid (12) | 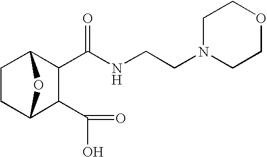 |
| N-Butanoic acid-7-Oxabicyclo(2.2.1)heptane-2,3- dicarboximide (13) | 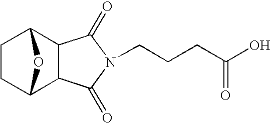 |
| N-Hexanoic acid-7-Oxabicyclo(2.2.1)heptane-2,3- dicarboximide (14) | 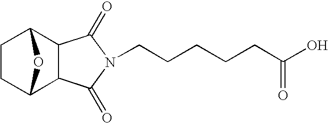 |
| N-Ethyl alcohol-7-Oxabicyclo(2.2.1)heptane-2,3- dicarboximide (15) | 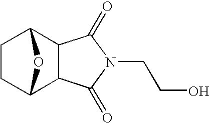 |
| N-2-Methoxypropyl-1-ol-7-Oxabicyclo(2.2.1)heptane- 2,3-dicarboximide (16) | 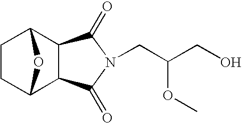 |
| 3-(methyl-3,4 dimethoxyphenyl carbamoyl)-7- Oxabicyclo(2.2.1)heptane-2-carboxylic acid (17) | 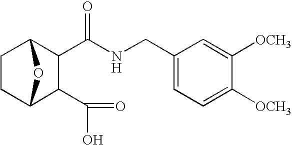 |
| 3-(Octanoic acid carbamoyl)-7- Oxabicyclo(2.2.1)heptane-2-carboxylic acid (18) | 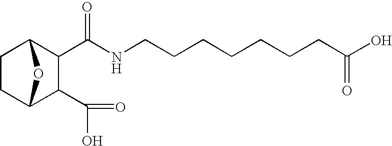 |
| 3-(4-hydroxyphenyl)-2-propionic acide)-7-Oxabicyclo (2.2.1)heptane-2,3-dicarboximide (19) | 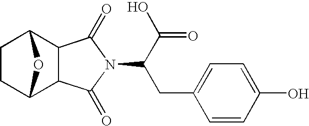 |
| exo-7-Oxabicyclo(2.2.1)heptane-2,3-dicarboximide (20) | 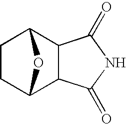 |

Compounds of the present invention may also be selected from compounds, or their salt or solvates thereof, and/or for use in accordance with the present invention include, but are not limited, to the following:

TABLE 2
Non-Limiting Examples of compounds of the present invention and/or
compounds useful in the practice of the methods of present invention.

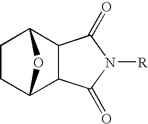

| Name | R |
|---|---|
| 4-Ethyl-7-Oxabicyclo(2.2.1)heptane-2,3-dicarboximide (9e) | 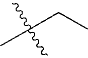 |
| 4-Sec-butyl-7-Oxabicyclo(2.2.1)heptane-2,3-dicarboximide (12a) | 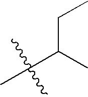 |
| 4-Hexyl-7-Oxabicyclo(2.2.1)heptane-2,3-dicarboximide (13a) | 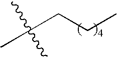 |
| 4-Cyclohexyl-7-Oxabicyclo(2.2.1)heptane-2,3-dicarboximide (14a) | 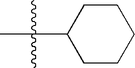 |
| 4-Dodecyl-7-Oxabicyclo(2.2.1)heptane-2,3-dicarboximide (17a) | 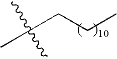 |
| 4-Tetradecyl-7-Oxabicyclo(2.2.1)heptane-2,3-dicarboximide (18a) | 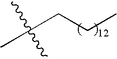 |
| 4-Octadecyl-7-Oxabicyclo(2.2.1)heptane-2,3-dicarboximide (19a) | 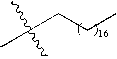 |
| 4-Allyl-7-Oxabicyclo(2.2.1)heptane-2,3-dicarboximide (20a) | 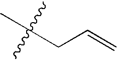 |
| 4-Oxiranylmethyl-7-Oxabicyclo(2.2.1)heptane-2,3-dicarboximide (22a) | 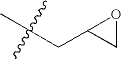 |
| 4-(3-Hydroxypropyl)-7-Oxabicyclo(2.2.1)heptane-2,3- dicarboximide (25a) | 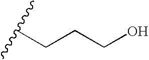 |
| 4-(6-Hydroxyhexyl)-7-Oxabicyclo(2.2.1)heptane-2,3- dicarboximide (26a) | 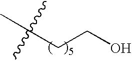 |
| 4-(2-Hydroxy-1-methylethyl)-7-Oxabicyclo(2.2.1)heptane-2,3- dicarboximide (27a) | 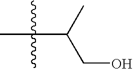 |
| 4-(2-Hydroxy-1,1-dimethylethyl)-7-Oxabicyclo(2.2.1)heptane- 2,3-dicarboximide (28a) | 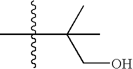 |
| 4-(1-Hydroxymethylpropyl)-7-Oxabicyclo(2.2.1)heptane-2,3- dicarboximide (29a) | 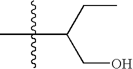 |
| 4-(3,5-Dioxo-10-oxa-4-azatricyclo[5.2.1]dec-4-yl)-butyric acid (30a) | 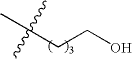 |
| 6-(3,5-Dioxo-10-oxa-4-azatricyclo[5.2.1]dec-4-yl)-hexanoic acid (31a) 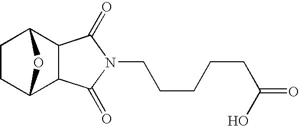 | 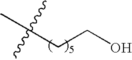 |
| 4-(2,3-Dihydroxypropyl)-10-oxa-4-azatricyclo[5.2.1]decane-3,5- dione (23a) ( 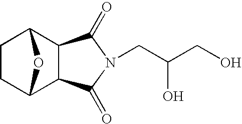 | 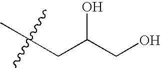 |
| 4-(3-Hydroxy-2-methoxypropyl)-10-oxa-4- azatricyclo[5.2.1]decane-3,5-dione (24a) | 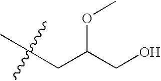 |
| 4-Morpholin-4-yl-7-Oxabicyclo(2.2.1)heptane-2,3-dicarboximide (32a) | 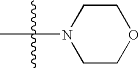 |
| 4-(2-Morpholin-4-ylethyl)-7-Oxabicyclo(2.2.1)heptane-2,3- dicarboximide dione (33a) | 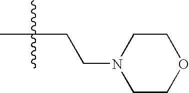 |
| 4-(3-Morpholin-4-ylpropyl)-7-Oxabicyclo(2.2.1)heptane-2,3- dicarboximide (34a) | 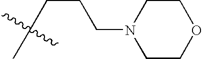 |
| 4-Phenyl-7-Oxabicyclo(2.2.1)heptane-2,3-dicarboximide (35a) | 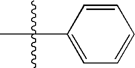 |
| 4-(4-Hydroxyphenyl)-10-oxa-4-azatricyclo[5.2.1]decane-3,5-dione | |
| (36a) | |
| 4-(4-Nitrophenyl)-7-Oxabicyclo(2.2.1)heptane-2,3-dicarboximide (37a) | 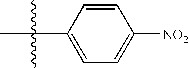 |
| 4-(3,5-Dioxo-10-oxa-4-azatricyclo[5.2.1]dec-4-yl)benzoic acid (38) | 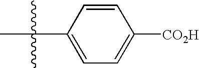 |
| 4-Benzyl-10-oxa-4-azatricyclo[5.2.1]decane-3,5-dione (39a) | 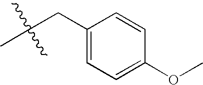 |
| 4-(4-Methoxybenzyl)-7-Oxabicyclo(2.2.1)heptane-2,3- dicarboximide (40a) | 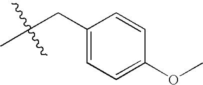 |
| 4-(3,4-Dimethoxybenzyl)-7-Oxabicyclo(2.2.1)heptane-2,3- dicarboximide (41a) | 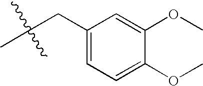 |
| 4-(2-Aminobenzyl)-7-Oxabicyclo(2.2.1)heptane-2,3-dicarboximide (42a) | 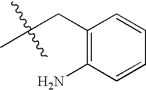 |
| Bis-3,6-epoxycyclohexane-1,2-dicarboximido)-trimethylene (Bis- | |
| norcantharimide-propyl linker) (43a) | |
| 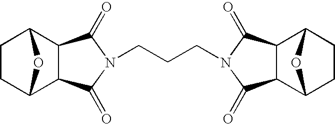 | |
| Bis-3,6-epoxycyclohexane-1,2-dicarboximido)-dodecylmethyle | |
| (Bis-norcantharimide-dodecyl linker (44a) | |
| 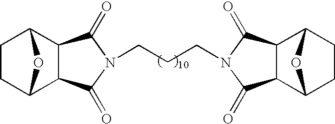 | |
| 3-[3-(Benzyloxy)pyridin-2-yl]-10-oxa-4-azatricyclo[5.2.1]decane- | |
| 3,5-dione (77) | |
| 4-{4-[2-(Dimethylamino)ethoxy]phenyl}-10-oxa-4- | |
| azatricyclo[5.2.1]decane-3,5-dione (78) | |
| 4,{2,[2-(Dimethylamino)ethoxy]phenyl}-10-oxa-4- | |
| azatricyclo[5.2.1]decane-3,5-dione (79) | |
| Compound (111) 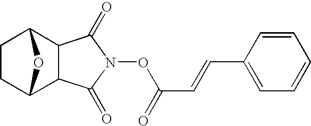 | 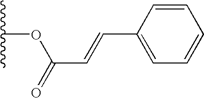 |
| Compound (112) | 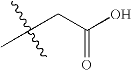 |
| Compound (115) 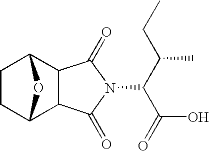 | 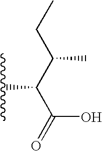 |
| Compound (116) 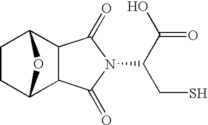 | 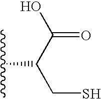 |
| Compound (117) 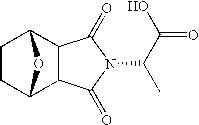 | 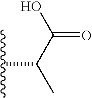 |
| Compound (121) 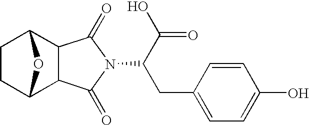 | 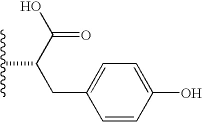 |
| Compound (123) 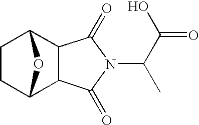 | 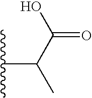 |
| Compound (124) 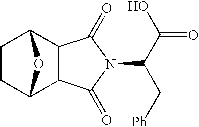 | 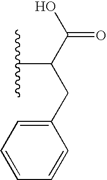 |
| Compound (126) 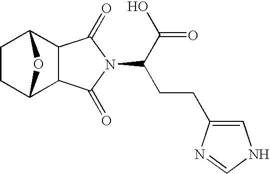 | 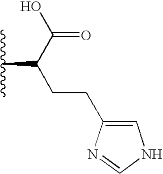 |
| Compound (129) 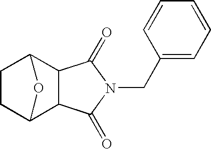 | 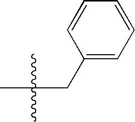 |

Compounds of the present invention and/or for use in the present invention may also be selected from compounds, or their salt or solvates thereof, include, but are not limited, to the following:

TABLE 3
Non-Limiting Examples of compounds of the present invention and/or
compounds useful in the practice of the methods of present invention.

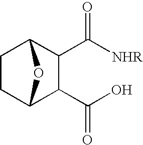

| Name | R |
|---|---|
| 3-(Butylcarbamoyl)-7-Oxabicyclo(2.2.1)heptane-2-carboxylic acid (9) | 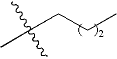 |
| 3-Propylcarbamoyl-7-oxabicyclol[2.2.1]heptane-2-carboxylic acid (9a) | 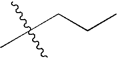 |
| 3-Hexylcarbamoyl-7-oxabicyclo[2.2.1]heptane-2-carboxylic acid (9b) | 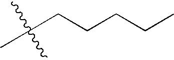 |
| 3-Octylcarbamoyl-7-oxabicyclo[2.2.1]heptane-2-carboxylic acid (9c) | 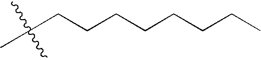 |
| 3-(Decylcarbamoyl)-7-Oxabicyclo(2.2.1)heptane-2-carboxylic acid (10) | 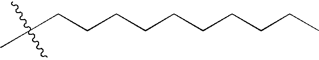 |
| 3-Dodecylcarbamoyl-7-oxabicyclo[2.2.1]heptane-2-carboxylic | |
| acid (10a) | |
| 3-Tetradecylcarbamoyl-7-oxabicyclo[2.2.1]heptane-2-carboxylic | |
| acid (10b) | |
| 3-(4-Benzylcarbamoyl)-7-Oxabicyclo(2.2.1)heptane-2-carboxylic | |
| acid (11) | |
| 3-(Nonylcarbamoyl)-7-Oxabicyc1o(2.2.1)heptane-2-carboxylic acid (10c) | 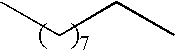 |
| 3-(Ethylmorpholine carbamoyl)-7-Oxabicyclo(2.2.1)heptane-2- | |
| carboxylic acid (12) | |
| 3-(methyl-3,4 dimethoxyphenyl carbamoyl)-7- | |
| Oxabicyclo(2.2.1)heptane-2-carboxylic acid (17) | |
| 3-(7-Carboxyheptylcarbamoyl)-7-oxabicyclo[2.2.1]heptane-2- | |
| carboxyhc acid (18) | |
| 3-(Octanoic acid carbamoyl)-7-Oxabicyclo(2.2.1)heptane-2- carboxylic acid (10) | 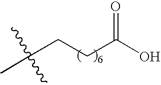 |
| 3-(4-hydroxyphenyl)-2-propionic acid)-7- | |
| Oxabicyclo(2.2.1)heptane-2,3-dicarboximide (19) | |
| 3-Octadecylcarbamoyl-7-oxabicyclo[2.2.1]heptane-2-carboxylic | |
| acid 21 | |
| 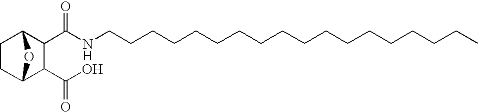 | |
| 3-Allylcarbamoyl-7-oxabicyclo[2.2.1]heptane-2-carboxylic acid (22) | 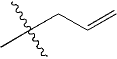 |
| 3-(3-Carboxpropylcarbamoyl)-7-oxabicyclo[2.2.1]heptane-2- carboxylic acid (23) | 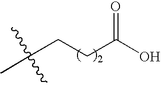 |
| 3-(5-Carboxypentylcarbamoyl)-7-oxabicyclo[2.2.1]heptane-2- carboxylic acid (24) 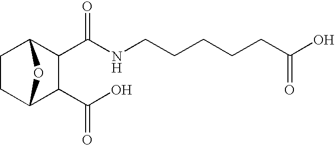 | 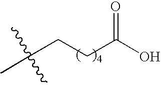 |
| 3-(6-Hydroxyhexylcarbamoyl)-7-oxabicyclo[2.2.1]heptane-2- carboxylic acid (26) | 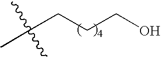 |
| 3-[2-(3H-Imidazol-4-yl)-ethylcarbamoyl]-7-Oxa- bicyclo[2.2.1]heptane-2-carboxylic acid (28) | 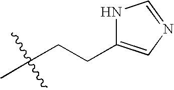 |
| 3-(4-Phenylcarbamoyl)-7-Oxabicyclo(2.2.1)heptane-2-carboxylic acid (32) | 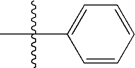 |
| 3-(2-Chlorophenylcarbamoyl)-7-Oxabicyclo(2.2.1)heptane-2- carboxylic acid (33) | 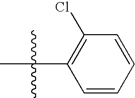 |
| 3-(3-Chlorophenylcarbamoyl)-7-Oxabicyclo(2.2.1)heptane-2- carboxylic acid (34) | 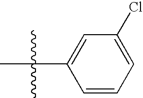 |
| 3-(4-Bromophenylcarbamoyl)-7-Oxabicyclo(2.2.1)heptane-2- carboxylic acid (35) | 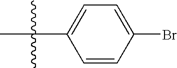 |
| 3-(3-Iodophenylcarbamoyl)-7-Oxabicyclo(2.2.1)heptane-2- carboxylic acid (36) | 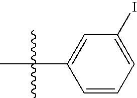 |
| 3-(4-Iodophenylcarbamoyl)-7-Oxabicyclo(2.2.1)heptane-2- carboxylic acid (37) | 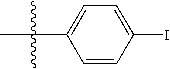 |
| 3-(4-Nitrophenylcarbamoyl)-7-Oxabicyclo(2.2.1)heptane-2- carboxylic acid (38) | 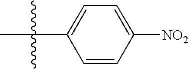 |
| 3-(2-hydroxyphenylcarbarnoyl)-7-Oxabicyclo(2.2.1)heptane-2- carboxylic acid (39) | 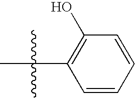 |
| 3-(4-Hydroxyphenyl carbamoyl)-7-Oxabicyclo(2.2.1)heptane-2- carboxylic acid (40) | 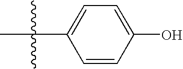 |
| 3-(4′-Benzoic acid carbamoyl)-7-Oxabicyclo(2.2.1)heptane-2- carboxylic acid (41) | 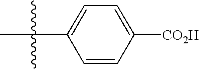 |
| 3-(4′-Methoxyphenyl carbamoyl)-7-Oxabicyclo(2.2.1)heptane-2- carboxylic acid (42) | 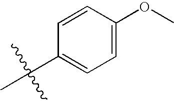 |
| 3-(4′-Methylthio benzene carbamoyl)-7-Oxabicyclo(2.2.1)heptane- 2-carboxylic acid (43) | 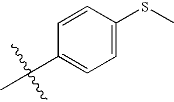 |
| 3-(2′-Methylthio benzene carbamoyl)-7-Oxabicyclo(2.2.1)heptane- 2-carboxylic acid (43) | 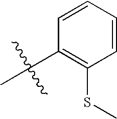 |
| 3-(4′-Pentyloxy benzene carbamoyl)-7-Oxabicyclo(2.2.1)heptane- 2-carboxylic acid (45) 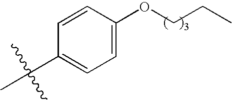 | |
| 3-(4′-Octylloxy benzene carbamoyl)-7-Oxabicyclo(2.2.1)heptane- 2-carboxylic acid (46) | 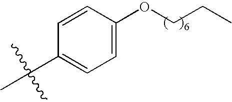 |
| 3-(2′-Benzyl alcohol carbamoyl)-7-Oxabicyclo(2.2.1)heptane-2- carboxylic acid (47) | 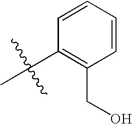 |
| 3-(4′-Benzyl alcohol carbamoyl)-7-Oxabicyclo(2.2.1)heptane-2- carboxylic acid (48) | 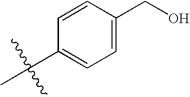 |
| 3-(3′-Benzyl alcohol carbamoyl)-7-Oxabicyclo(2.2.1)heptane-2- carboxylic acid (49) | 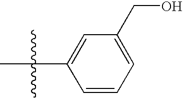 |
| 3-(4′-Ethylene benzene carbamoyl)-7-Oxabicyclo(2.2.1)heptane-2- carboxylic acid (50) | 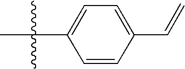 |
| 3-(3′-Ethynyl benzene carbamoyl)-7-Oxabicyclo(2.2.1)heptane-2- carboxylic acid (51) | 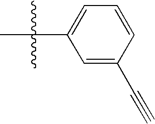 |
| 3-(4′-Benzene acetic acid carbamoyl)-7-Oxabicyclo(2.2.1)heptane- 2-carboxylic acid (52) | 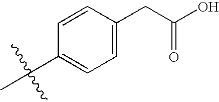 |
| 3-(3′-Benzene acetic acid carbamoyl)-7-Oxabicyclo(2.2.1)heptane- 2-carboxylic acid (53) | 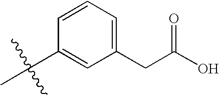 |
| 3-(4′-Benzene propanoic acid carbamoyl)-7- Oxabicyclo(2.2.1)heptane-2-carboxylic acid (54) | 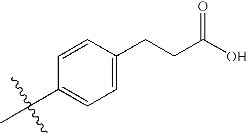 |
| 3-(4′-Morpholino-benzene carbamoyl)-7- Oxabicyclo(2.2.1)heptane-2-carboxylic acid (55) | 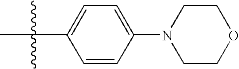 |
| 3-(2′-Ethyl-phenylcarbamoyl)-7-oxa-bicyclol[2.2.1]heptane-2- carboxylic acid (57) | 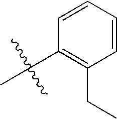 |
| 3-(2′,6′-Dimethylphenylcarbamoyl)7-oxa-bicyclo[2.2.1]heptane-2- carboxylic acid (58) | 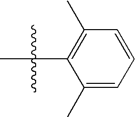 |
| 3-(2′,4′-Dimethylphenylcarbamoyl)7-oxa-bicyclo[2.2.1]heptane-2- carboxylic acid (59) | 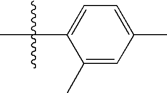 |
| 3-(2′,3′-Dimethylphenylcarbamoyl)7-oxa-bicyclo[2.2.1]heptane-2- carboxylic acid (60) | 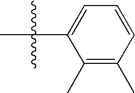 |
| 3-(2′,4′,6′-Trimethylphenylcarbamoyl)7-oxa- bicyclo[2.2.1]heptane-2-carboxylic acid (61) | 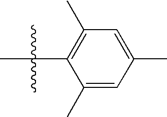 |
| 3-(3′,5′-Di-tert-butylphenylcarbamoyl)7-oxa- bicyclo[2.2.1]heptane-2-carboxylic acid (62) | 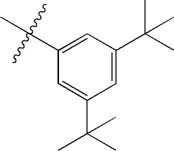 |
| 3-(2′-Naphthylcarbamoyl)7-oxa-bicyclo[2.2.1]heptane-2- carboxylic acid (63) | 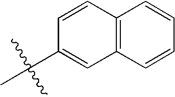 |
| Compound (64) | 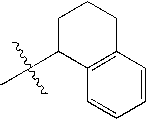 |
| 3-[(Pyridin-2-ylmethyl)-carbamoyl]-7-oxa-bicyclo[2.2.1]heptane- 2-carboxylic acid (65) ( | 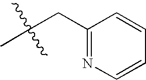 |
| 3-(Benzylcarbamoyl)-7-oxa-bicyclo[2.2.1]heptane-2-carboxylic acid (66) | 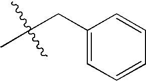 |
| 3-(4′-Methoxy-benzylcarbamoyl)-7-oxa-bicyclo[2.2.1]heptane-2- carboxylic acid (67) | 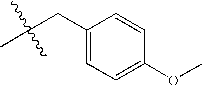 |
| 3-(3′,4′-Dimethoxy-benzylcarbamoyl)-7-oxa- bicylo[2.2.1]heptane-2-carboxylic acid (68) | 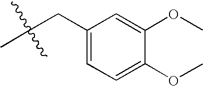 |
| 3-(4′-Chlorobenzylcaramoyl)-7-oxa-bicyclo[2.2.1]heptane-2- carboxylic acid (69) | 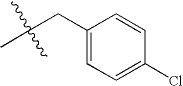 |
| 3-(Phenethylcarbamoyl)-7-oxa-bicyclo[2.2.1]heptane-2-carboxylic acid (70) | 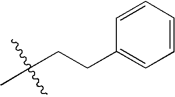 |
| 3-(Phenyl-propylcarbamoyl)-7-oxa-bicyclo[2.2.1]heptane-2- carboxylic acid (71) | 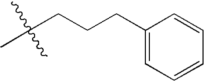 |
| 3-(Phenyl-butylcarbamoyl)-7-oxa-bicyclo[2.2.1]heptane-2- carboxylic acid (72) | 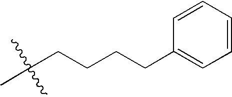 |
| 3-(4′-Carboxybenzylcarbamoyl)-7-oxa-bicyclo[2.2.1]hepane-2- carboxylic acid (73) | 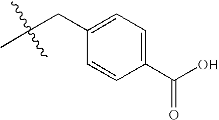 |
| Compound (74) | 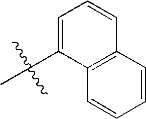 |
| 3-(1-benzylpiperidin-4-ylcarbamoyl]-7-oxabicyclo[2.2.1]heptane- | |
| 2-carboxylic acid (75) | |
| 3-(4-Carboxy-3-chlorophenylcarbamoyl)-7- | |
| oxabicyclo[2.2.1]heptane-2-carboxylic acid (76) | |
| 3-[(1-Methyl-1H-indol-4-yl)methylcarbamoyl]-7- | |
| oxabicyclo[2.2.1]heptane-2-carboxylic acid (80) | |
| 3,[(6,7-Dihydro-4H-thieno[3,2-c]pyran-4-yl)methylcarbamoyl]-7- | |
| oxabicyclo[2.2.1]heptane-2-carboxylic acid (81) | |
| 3-(Benzo[d]thiazol-5-ylcarbamoyl)-7-oxabicyclo[2.2.1]heptane-2- | |
| carboxylic acid (82) | |
| 3-Octylcarbamoyl-7-oxabicyclo[2.2.1]heptane-2-carboxylic acid | |
| (83) | |
| 3-Decylcarbamoyl-7-oxabicyclo[2.2.1]heptane-2-carboxylic acid | |
| (85) | |
| 3-Allylcarbamoyl-7-oxabicyclo[2.2.1]heptane-2-carboxylic acid | |
| (86) | |
| 3-(4-hydroxyphenylcarbamoyl)-7-oxabicyclo[2.2.1]heptane-2- | |
| carboxylic acid (87) | |
| 3-(2-Ethylphenylcarbamoyl)-7-oxa-bicyclo[2.2.1]heptane-2- | |
| carboxylic acid (88) | |
| Compound (89) | 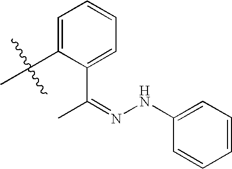 |
| 3-[4-(Ethoxycarbonyl)-1H-pyrazol-3-ylcarbamoyl]-7- | |
| oxabicyclo[2.2.1]heptane-2-carboxylic acid (90) | |
| 3-(4-tert-Butylphenylcarbamoyl)-7-oxabicyclo[2.2.1]heptane-2- carboxylic acid (91) | 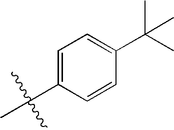 |
| 3-(2,4-Di-tert-Butylphenylcarbamoyl)-7-oxabicyclo[2.2.1]heptane- 2-carboxylic acid (92) | 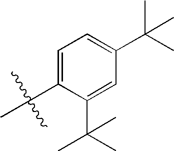 |
| 3-(2-Dimethylaminoethylcarbamoyl)-7-oxabicyclo[2.2.1]heptane- 2-carboxylic acid (93) | 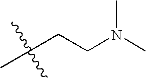 |
| 3-(3-Dimethylaminopropylcarbamoyl)-7- oxabicyclo[2.2.1]heptane-2-carboxylic acid (94) | 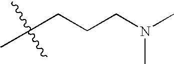 |
| 3-(2-Morpholin-4-ylethylcarbamoyl)-7-oxabicyclo[2.2.1]heptane- 2-carboxylic acid (95) | 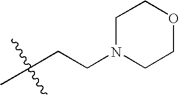 |
| 3-(3,4-Difluorophenylcarbamoyl)-7-oxabicyclo[2.2.1]heptane-2- carboxylic acid (96) | 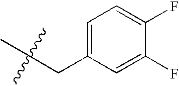 |
| 3-(4-Trifluoromethylphenylcarbamoyl)-7- oxabicyclo[2.2.1]heptane-2-carboxylic acid (97) | 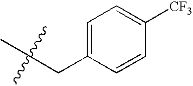 |
| Compound (98) | 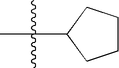 |
| Compound (99) | 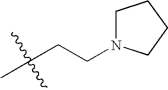 |
| Compound (100) | 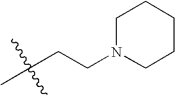 |
| Compound (102) | 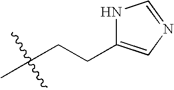 |
| Compound (103) | 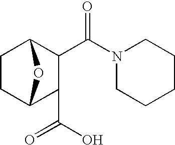 |
| Compound (104) | 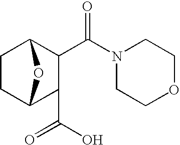 |
| Compound (105) | 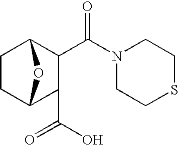 |
| Compound (106) | 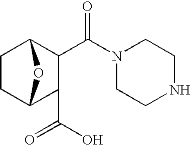 |
| Compound (107) | 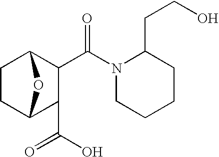 |
| Compound (108) | 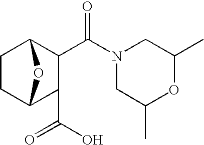 |
| Compound (109) | 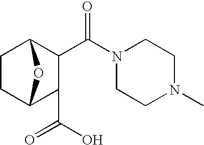 |
| Compound (110) | 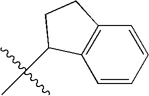 |
| Compound (118) 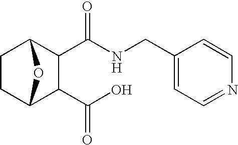 | 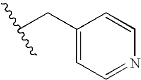 |
| Compound (119) 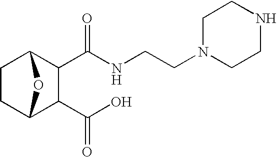 | 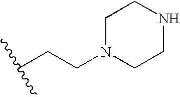 |
| Compound (120) | |
| 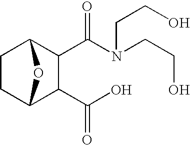 | |
| Compound (128) 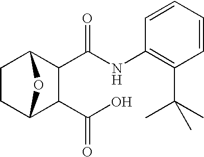 | 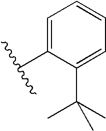 |
| Compound (129) | |
| 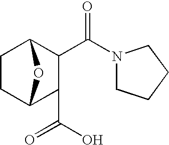 | |
| Compound (130) 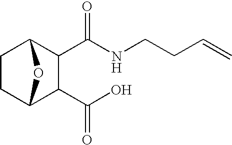 | 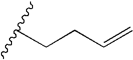 |

Compounds of the present invention may also be selected from compounds, or their salt or solvates thereof, and/or for use in accordance with the present invention include, but are not limited, to the following:

TABLE 4
Non-Limiting Examples of compounds of the present invention and/or
compounds useful in the practice of the methods of present invention.

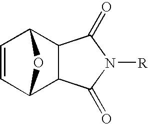

| Name | R |
|---|---|
| Compound (113) | H |
| 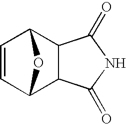 | |
| Compound (114) 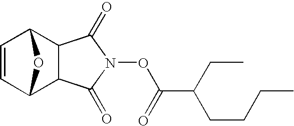 | 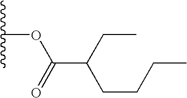 |
| 4-Hexyl-7-Oxabicyclo(2.2.1)heptane-2,3-dicarboximide (13a) | 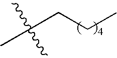 |
| Compound (122) 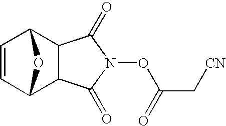 | 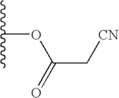 |
| Compound (125) | |
| 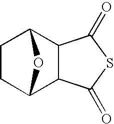 | |
| Compound (127) | |
| 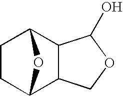 | |

The compounds described in the tables above maybe used in the methods of the present invention.

In particular, the pesent invention provides a method for controlling invertebrate pest(s), comprising contacting the invertebrate pest(s) with at least one invertebrate control agent(s) selected from the compound(s) described in the tables above.

In yet another aspect, the present invention provides a pharmaceutical and/or veterinary composition comprising a pharmaceutically and/or veterinarily acceptable carrier or diluent together with at least one invertebrate control agent(s) as specified above.

In yet a further aspect, the present invention provides a composition for controlling an invertebrate pest comprising an effective pest-controlling amount of at least one compound as specified above and at least one additional component selected from the group consisting of surfactants, solid diluents and liquid diluents.

The present inventors have also identified polypeptides, and polynucleotides encoding thereof, which can be used as targets for controlling invertebrates.

Thus, in yet another aspect, the present invention provides a substantially purified and/or recombinant polypeptide selected from:
i) a polypeptide comprising an amino acid sequence as provided in SEQ ID NO:1,
ii) a polypeptide comprising an amino acid sequence which is at least 60% identical to SEQ ID NO:1, and
iii) a biologically active fragment of i) or ii),

wherein the polypeptide has phosphatase activity.

Preferably, the polypeptide can be purified from an invertebrate. More preferably, the polypeptide can be purified from a Nematode. More preferably, the polypeptide can be purified from a species of the Genus Trichostrongylus.

In an embodiment, the polypeptide is a fusion protein further comprising at least one other polypeptide sequence.

In yet another aspect, the present invention provides an isolated and/or exogenous polynucleotide comprising a sequence of nucleotides selected from:
i) a sequence of nucleotides as provided in SEQ ID NO:2,
ii) a sequence of nucleotides encoding a polypeptide of the invention,
iii) a sequence of nucleotides which is at least 60% identical to SEQ ID NO:2, and
iv) a sequence which hybridises to any one of i) to iii) under stringent conditions.

In a further aspect, the present invention provides an oligonucleotide which comprises at least 19 contiguous nucleotides of a polynucleotide of the invention.

Preferably, the oligonucleotide comprises at least 19 contiguous nucleotides of SEQ ID NO:2.

In yet a further aspect, the present invention provides a polynucleotide which, when present in a cell of an invertebrate, down-regulates the level of phosphatase activity in the cell when compared to a cell that lacks said polynucleotide. Examples of such polynucleotides include, but are not limited to, an antisense polynucleotide, a sense polynucleotide, a catalytic polynucleotide, a microRNA and a double stranded RNA.

In an embodiment, the polynucleotide is a catalytic polynucleotide capable of cleaving a polynucleotide encoding a polypeptide of the invention.

In another embodiment, the polynucleotide is a double stranded RNA (dsRNA) molecule comprising an oligonucleotide of the invention, wherein the portion of the molecule that is double stranded is at least 19 basepairs in length and comprises said oligonucleotide.

Preferably, the dsRNA is expressed from a single promoter, wherein the strands of the double stranded portion are linked by a single stranded portion.

Also provided is a vector comprising and/or encoding the polynucleotide of the invention. Preferably, the polynucleotide is operably linked to a promoter.

In yet a further aspect, the present invention provides a host cell comprising a vector of the invention, and/or a polynucleotide of the invention.

In another aspect, the present invention provides a method of making the polypeptide of the invention, comprising the steps of:

(a) inserting the nucleotide sequence of the invention in an expression vector such that said nucleotide sequence is operably linked to a promoter; and

(b) introducing said expression vector in to a host cell whereby said host cell produces the polypeptide encoded by said nucleotide sequence.

Preferably, the method further comprises the step of isolating said polypeptide.

Also provided is a non-human transgenic organism comprising an exogenous polynucleotide of the invention.

Preferably, the non-human transgenic organism comprises a cell of the invention.

In an embodiment, the antisense polynucleotide, catalytic polynucleotide or dsRNA down-regulates the production of a polypeptide of the invention which is endogenously produced by the organism or a parasite thereof.

In another aspect, the present invention provides a substantially purified antibody, or fragment thereof, that specifically binds a polypeptide of the invention.

In another aspect, the present invention provides a method of identifying an invertebrate control agent, the method comprising

a) exposing a polypeptide of the invention to a candidate agent, and

b) assessing the ability of the candidate agent to modulate the activity of the polypeptide.

Typically, the activity is phosphatase activity. Examples of assays for detecting phosphatase activity are decsribed herein.

In yet a further aspect, the present invention provides a method of identifying an invertebrate control agent, the method comprising

a) exposing a polypeptide of the invention to a binding partner which binds the polypeptide, and a candidate agent, and

b) assessing the ability of the candidate agent to compete with the binding partner for binding to the polypeptide.

In a preferred embodiment, the binding partner is a protein which can be dephosphorylated by a polypeptide of the invention.

Preferably, the binding partner is detectably labeled.

In a further aspect, the present invention provides a method of identifying an invertebrate control agent, the method comprising using the structural coordinates of a crystal of a polypeptide of the invention to computationally evaluate a candidate agent for its ability to bind to the polypeptide, and testing the candidate agent identified for its ability to modulate the activity of the polypeptide.

Preferably, the agent is an antagonist of the polypeptide.

In yet another aspect, the present invention provides a method of identifying an invertebrate control agent, the method comprising

a) exposing a polynucleotide encoding a polypeptide of the invention to a candidate agent under conditions which allow expression of the polynucleotide, and

b) assessing the ability of the candidate agent to modulate levels of polypeptide produced by the polynucleotide.

In another aspect, the present invention provides a method of identifying an invertebrate control agent, the method comprising

a) exposing a polynucleotide encoding a polypeptide of the invention to a candidate agent, and

b) assessing the ability of the candidate agent to hybridize and/or cleave the polynucleotide.

In a further aspect, the present invention provides an invertebrate control agent identified using a method of the invention.

In yet a further aspect, the present invention provides a composition comprising the polypeptide of the invention, a polynucleotide of the invention, the vector of the invention, the host cell of the invention, the antibody of the invention and/or the invertebrate control agent of the invention, and an acceptable carrier.

In another aspect, the present invention provides a method of controlling an invertebrate organism, the method comprising contacting the organism with a polynucleotide of the invention, the vector of the invention, the host cell of the invention, the antibody of the invention and/or the invertebrate control agent of the invention and/or the composition of the invention.

In a further aspect, the present invention provides a method of treating and/or preventing a disease in a subject caused by an invertebrate, the method comprising administering to the subject a polynucleotide of the invention, the vector of the invention, the host cell of the invention, the antibody of the invention and/or the invertebrate control agent of the invention and/or the composition of the invention.

In yet another aspect, the present invention provides for the use of a polynucleotide of the invention, the vector of the invention, the host cell of the invention, the antibody of the invention and/or the invertebrate control agent of the invention and/or the composition of the invention for the manufacture of a medicament for treating and/or preventing a disease in a subject caused by an invertebrate.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1 Nucleotide sequence of the cDNA of the Trichostrongylus vitrinus serine/threonine phosphatase gene (Tv-stp1) and its predicted amino acid sequence. The nucleotide sequence determined from the original EST Tvm02_C07 is in bold text (accession no. NP_{—}498617.1). The untranslated regions are in lower-case and the protein coding nucleotide sequence is in upper-case. ATG and TGA (asterisk) are the inferred translation initiation and termination signals, respectively (TGA); the putative polyadenylation signal sequence is underlined. The amino acid residues representing the serine/threonine specific protein phosphatase protein pattern PS00125, [LIVMN]-[KR]-G-N-H-E, are shaded.

FIG. 2 Alignment of the inferred amino acid sequence (Tv-STP1) for the Trichostrongylus vitrinus serine/threonine phosphatase with selected those predicted from Caenorhabditis elegans genes C47A4.3, C09H_{5.7}, T03F1.5, WO9C3.6, F56C9.1 and F29F11.6 (Ce; accession nos. CAB62794, AAB65386, NP_{—}491237, AAC24414, NP_{—}001022616 and CAA98273, respectively) and from Oesophagostomum dentatum (accession no. AF496634); Trypanosoma brucei (Tb; accession no. EAN79771); Debaryomyces hansenii (Dh; accession no. CAG87702); Nicotiana tabacum (Nt; accession no. CAB078703) and Homo sapiens (Hs; accession no. AAV38549). Amino acid predicted to be involved in the catalytic pocket (metal binding) of the enzyme are indicated by asterisks, and the histidine residue predicted to be involved in the active site is indicated with an arrow. Dashes indicate gaps in the sequence, included for alignment purposes. The Prosite motif PS00125 is indicated with a bar.

FIG. 3 Phylogenetic relationships of serine/threonine phosphatases from all organisms listed in Table 3 (panel A) and nematodes only (panel B). Neighbor-joining trees displayed, with bootstrap values exceeding % indicated above the branches. Bootstrap values obtained for concordant branches using the maximum parsimony method (100 replicates) are indicated below the branches.

FIG. 4 Diagrammatic representation of the genomic organization of Tv-stp1 compared with the full-length C. elegans genes C47A4.3, C09H5.7, T03F1.5, WO9C3.6, F56C9.1 and F29F11.6 (Ce; accession nos. Z82263, AF016433, U88169, U88178, U00063, Z73974, respectively) and from O. dentatum (Od-mpp1; accession no. AF496634; Boag et al., 2003). The structures of the C. elegans genes were determined based on the comparisons and alignments with their ‘expressed sequence tags’ (ESTs) (see WormBase at http://www.wormbase.org/). Grey rectangles represent exons. The lines joining the exons indicate introns. Numbers above the gene refer to lengths of introns and those below refer to the lengths of exons determined based on comparison with respective cDNA sequences (cf. FIG. 1). Drawn to scale.

FIG. 5 Analysis of transcription of Tv-stp1 in different developmental stages and both sexes of Trichostrongylus vitrinus by reverse-transcription PCR, including a positive control PCR using primers for the amplification of a region (187 bp) from the large subunit (LSU) of nuclear ribosomal RNA. Developmental stages of T. vitrinus were the first- to fourth-stage larvae (L1-L4; mixed sexes) as well as male and female adults (AM and AF, respectively). A transcript representing the full-length cDNA of Tv-stp1 (951 bp; see FIG. 1) was amplified exclusively from the adult male of T. vitrinus using primer set STALLF3-STALLR3; no other products were amplified from any of the samples using these primers, inferring the absence of a transcript and of genomic DNA in the samples. Genomic DNA (G) and no-DNA (N) controls included.

FIG. 6 illustrates the synthetic scheme for the synthesis of the 7-Oxabicyclo(2.2.1)heptane-2 dicarboximide derivatives and 7-Oxabicyclo(2.2.1)heptane-2-carboxylic acid derivatives.

FIG. 7 illustrates the synthetic scheme for the synthesis of epoxide derivatives.

FIG. 8 shows the loading of 96-well microtitre plate for primary screening of forty-five compounds at highest concentration of 5 mM to kill all T. vitrinus larvae added to the well.

FIG. 9 shows the results of the screening, where each well was scored for the % of T. vitrinus L3 larvae killed by the compound present. +++=100% killed, ++=75% killed, +=50% killed, −=< or equal to 25% killed, C=the compound formed crystals or a precipitate and was not scored.

FIG. 10 shows the loading of 96-well microtitre plate for secondary screening of eight compounds from the primary screening which appeared to kill all T. vitrinus L3 larvae at a concentration of 5 mM. Sequential ten-fold dilutions of 5 mM, down to 0.5 μM, were screened for the ability to kill all larvae added to the well. This allowed direct comparisons of toxicity between compounds.

FIG. 11 shows the secondary screening results where each well was scored for the % of T. vitrinus L3 larvae killed by the compound present. +++=100% killed, ++=75% killed, +=50% killed, −=< or equal to 25% mortality, C=the compound formed crystals or a precipitate and was not scored.

FIG. 12 shows the loading of Plate 1 for primary screening of various compounds at highest concentration of 5 mM to kill H. contortus L3 larvae added to the well.

FIG. 13 shows the loading of Plate 2 for primary screening of various compounds at highest concentration of 5 mM to kill H. contortus L3 larvae added to the well.

FIG. 14 shows the results of the screening of Plate 1 and Plate 2, where each well was scored for the % of H. contortus L3 larvae killed by the compound present, where −=<10% of larvae killed, ++=50% killed, +++=90% killed, ++++=all larvae killed, C=the compound crystallized, P=the compound formed a precipitate.

FIG. 15 shows the loading of Plate 3 for secondary screening of compounds which appeared to kill all H. contortus L3 larvae at a concentration of 5 mM from the primary screening. Sequential ten-fold dilutions of 5 mM, down to 0.5 μM, were screened for the ability to kill all larvae added to the well. This allowed direct comparisons of toxicity between compounds.

FIG. 16 shows the loading of Plate 4 for secondary screening of compounds which appeared to kill all H. contortus L3 larvae at a concentration of 5 mM from the primary screening. Sequential ten-fold dilutions of 5 mM, down to 0.5 μM, were screened for the ability to kill all larvae added to the well. This allowed direct comparisons of toxicity between compounds.

FIG. 17 shows the secondary screening results of Plate 3 and Plate 4 where each well was scored for the % of H. contortus L3 larvae killed by the compound present, where −=<10% of larvae killed, ++=50% killed, +++=90% killed, ++++=all larvae killed, C=the compound crystallized, P=the compound formed a precipitate.

FIG. 18 shows the loading of Plate 5 for primary screening of various compounds at highest concentration of 5 mM to kill Trichostrongylus colubriformis L3 larvae added to the well.

FIG. 19 shows the loading of Plate 6 for primary screening of various compounds at highest concentration of 5 mM to kill Trichostrongylus colubriformis L3 larvae added to the well.

FIG. 20 shows the secondary screening results of Plate 5 and Plate 5 where each well was scored for the % of Trichostrongylus colubriformis L3 larvae killed by the compound present, C=the compound crystallized, P=the compound formed a precipitate.

### KEY TO THE SEQUENCE LISTING

SEQ ID NO:1—Tv-stp1 polypeptide from Trichostrongylus vitrinus.
SEQ ID NO:2—Open reading frame encoding Tv-stp1 polypeptide from Trichostrongylus vitrinus.
SEQ ID NO:3—cDNA encoding Tv-stp1 polypeptide from Trichostrongylus vitrinus.
SEQ ID NO:4—Phosphatase from Caenorhabditis elegans (GenBank Accession No. CAB62794).
SEQ ID NO:5—Phosphatase from Caenorhabditis elegans (GenBank Accession No. AAB65386).
SEQ ID NO:6—Phosphatase from Caenorhabditis elegans (GenBank Accession No. NP 491237).
SEQ ID NO:7—Phosphatase from Caenorhabditis elegans (GenBank Accession No. AAC24414).
SEQ ID NO:8—Phosphatase from Caenorhabditis elegans (GenBank Accession No. NP_{—}001022616).
SEQ ID NO:9—Phosphatase from Caenorhabditis elegans (GenBank Accession No. CAA98273).
SEQ ID NO:10—Phosphatase from Oesophagostomum dentatum (GenBank Accession No. AF496634).
SEQ ID NO:11—Phosphatase from Trypanosoma brucei (GenBank Accession No. EAN79771).
SEQ ID NO:12—Phosphatase from Debaryomyces hansenii (GenBank Accession No. CAG87702).
SEQ ID NO:13—Phosphatase from Nicotiana tabacum (GenBank Accession No. CAB07873).
SEQ ID NO:14—Phosphatase from Homo sapiens (GenBank Accession No. AAV38549).
SEQ ID NO's 15 to 20—Oligonucleotide primers.
SEQ ID NO:21—siRNA.

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, molecular genetics, invertebrate control biology, chemistry, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### DEFINITIONS

Listed below are definitions of various terms used to describe this invention. These definitions apply to the terms as they are used throughout this specification and claims, unless otherwise limited in specific instances, either individually or as part of a larger group.

The term “independently selected” is used herein to indicate that the choices can be identical or different. In the case of R groups, for example, the term “independently selected” indicates that the R groups (e.g., R′, R², R³) can be identical (e.g., R¹, R² and R³ all may be substituted alkyl groups) or different (e.g., R′ and R² may be substituted alkyl groups and R³ may be an aryl group).

An “aliphatic group,” as used herein, is an acyclic, non-aromatic moiety that may contain any combination of carbon atoms, hydrogen atoms, halogen atoms, oxygen, nitrogen, sulfur, phosphorus or other atoms, and optionally contain one or more units of unsaturation, e.g., double and/or triple bonds. An aliphatic group may be straight chained, or branched and preferably contains between about one and about 20 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but are not limited to, cyclopropyl, cyclobutyl, cyclopentyl, cyclohexyl, bicyclo [2.2.1]heptyl, bicyclo [2.2.2]octyl and the like.

The term “substituted alicyclic,” as used herein, refers to an alicyclic group as previously defined, substituted by one, two, three or more substituents.

The term “alkyl,” as used herein, refers to saturated, straight chain or branched hydrocarbon moieties containing up to twenty carbon atoms. The terms “C₁₋₂₀ alkyl” and “C₃₋₁₀ alkyl,” as used herein, refer to saturated, straight chain or branched hydrocarbon moieties containing one, to twenty carbon atoms and three to ten carbon atoms respectively. Examples of alkyl groups include, but are not limited to, methyl, ethyl, propyl, isopropyl, n-hexyl, octyl, decyl, dodecyl and the like.

The terms “aryl” or “aromatic,” as used herein, refer to a mono- or polycyclic carbocyclic ring system having one or more aromatic rings. Examples of aryl groups include, but not limited to, phenyl, naphthyl, tetrahydronaphthyl, indanyl, idenyl and the like.

The terms “substituted aryl” or “substituted aromatic,” as used herein, refer to an aryl or aromatic group as previously defined substituted by one, two, three or more substituents.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, allylamino, acylamino, alkylthio, alkylsulfinyl, and alkylsulfonyl. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The terms “heterocyclic,” or “heterocycloalkyl” as used herein, refer to a non-aromatic ring, comprising three or more ring atoms, or a bi- or tri-cyclic fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Examples of heterocyclic groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like.

The term “substituted heterocyclic,” as used herein, refers to a heterocyclic group, as previously defined, substituted by one, two, three or more substituents.

The term “saturated” refers to lack of double and triple bonds between atoms of a radical group such as ethyl, cyclohexyl, pyrrolidinyl, and the like.

The term “unsaturated” refers to the presence one or more double and triple bonds between atoms of a radical group such as vinyl, acetylenyl, oxazolinyl, cyclohexenyl, acetyl and the like.

The synthesized compounds can be separated from a reaction mixture and further purified by a method such as column chromatography, high pressure liquid chromatography, precipitation, or recrystallization. Further methods of synthesizing the compounds of the formulae herein will be evident to those of ordinary skill in the art. Additionally, the various synthetic steps may be performed in an alternate sequence or order to give the desired compounds.

Compounds of the invention can exist as one or more stereoisomers. The various stereoisomers include enantiomers, diastereomers, atropisomers and geometric isomers. A person skilled in the art will appreciate that one stereoisomer may be more active and/or may exhibit beneficial effects when enriched relative to the other stereoisomer(s) or when separated from the other stereoisomer(s). Additionally, the person skilled in the art knows how to separate, enrich, and/or to selectively prepare said stereoisomers. Accordingly, the compounds of the invention may be present as a mixture of stereoisomers, individual stereoisomers, or as an optically active form.

The terms “pest controlling amount” or “controlling an invertebrate pest,” used throughout the specification and claims, are meant to include any pesticidal (killing) or pestistatic (preventing the host plant or host animal from being eaten, or inhibiting, maiming or generally interfering) activities of a composition against a given pest at any stage in its life cycle. Thus, these terms not only include killing, but also include the production of behavioural abnormalities (e.g., tremor, inco-ordination, hyperactivity, anorexia, leaf walk-off behaviour) which interfere with activities such as, but note limited to, eating, molting, hatching, mobility or plant attachment. The terms also include chemosterilant activity which produces sterility in insects by preventing the production of ova or sperm, by causing death of sperm or ova, or by producing severe injury to the genetic material of sperm or ova, so that the larvae that are produced do not develop into mature progeny.

As used herein, the terms “control”, “controlling” or variations thereof refer to a sufficient reduction in the invertebrate density and maintenance of that density over a period of time and/or prevention of an increase in invertebrate density and/or delay in the increase of invertebrate density.

“Treatment” or “treating” as used herein means any therapeutic intervention in a subject, usually a mammalian subject, including:
(i) prevention, that is, causing the clinical symptoms not to develop, e.g., preventing infection and/or preventing progression to a harmful state;
(ii) inhibition, that is, arresting the development or further development of clinical symptoms, e.g., mitigating or completely inhibiting an active (ongoing) infection so that pathogen load is decreased to the degree that it is no longer harmful, which decrease can include complete elimination of an infectious dose of the pathogen from the subject; and/or
(iii) relief, that is, causing the regression of clinical symptoms e.g., causing a relief of fever, inflammation, and/or other symptoms caused by an infection.

The term “effective amount” or “therapeutically effective amount” means a dosage sufficient to provide for treatment for the disease state being treated or to otherwise provide the desired effect (e.g., induction of an effective immune response). The precise dosage will vary according to a variety of factors such as subject-dependent variables (e.g., age, immune system health, etc.), the disease (e.g., the species of the infecting pathogen), and the treatment being effected. In the case of an intracellular pathogen infection, an “effective amount” is that amount necessary to substantially improve the likelihood of treating the infection, in particular that amount which improves the likelihood of successfully preventing infection or eliminating infection when it has occurred.

The compositions of the invention may be formulated together with an acceptable carrier or diluent as required. In a preferred form, the carrier or diluent is a pharmaceutically and/or veterinarary acceptable carrier or diluent.

The term “pharmaceutically carrier or diluent” and/or “a veterinarily acceptable carrier or diluent” as used herein means a pharmaceutically and/or veterinarily acceptable material, composition or vehicle, such as a liquid or solid filler, diluent, excipient, solvent or encapsulating material, involved in carrying or transporting the subject compounds from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable and/or veterinarily acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as ethylene glycol, propylene glycol; polyols, such as glycerin, sorbitol, mannitol and polyethylene glycol; esters, such as ethyl oleate and ethyl laurate; agar; buffering agents, such as magnesium hydroxide and aluminium hydroxide; alginic acid; pyrogen-free water; isotonic saline; Ringer's solution; ethyl alcohol; phosphate buffer solutions; and other non-toxic compatible substances employed in pharmaceutical and or veterinary formulations.

By “phosphatase activity” we mean a polypeptide that catalyses the removal of a phosphate group from a substrate. Phosphatase activity may be assessed by any technique known in the art. Such techniques are described and explained in Gupta et al. (1997) and Hart et al. (2004). Furthermore, commercial kits for testing phosphatase activity are avilable, for example from Promega.

### Invertebrates

By “invertebrate” we mean any animal that lacks a backbone or spinal column. They include but are not limited to the protozoans, annelids, cnidarians, echinoderms, flatworms, nematodes, molluscs, and arthropods. In a preferred embodiment, the invertebrate is a nematode.

Invertebrates can be further divided into “free living” versus “non-free living.” Non-free living invertebrates, often called parasites, live in or on a host organism and derive subsistence from it without rendering it any service in return. In a preferred embodiment, the invertebrate is non-free living. More preferably, the invertebrate is non-free living nematode.

In a preferred embodiment the invertebrate is selected from the genus Trichostrongylus, including Trichostrongylus axei, Trichostrongylus capricola, Trichostrongylus colubriformis, Trichostrongylus probolurus, Trichostrongylus retortaeformis, Trichostrongylus rugatus, Trichostrongylus tenuis and Trichostrongylus vitrinus.

In a further preferred embodiment the invertebrate is selected from the genus Haemonchus, including Haemonchus contortus, Haemonchus longistipes, Haemonchus placei, Haemonchus similis.

In a further preferred embodiment the invertebrate is selected from the genus Ostertagia/Teladorsagia, including Ostertagia arctica, Ostertagia dikmansi, Ostertagia gruehneri, Ostertagia kokhida, Ostertagia leptospicularis, Ostertagia lyrata, Ostertagia mossi, Ostertagia nianqingtanggulansis, Ostertagia ostertagi and Ostertagia trifurcata.

In a further preferred embodiment the invertebrate is selected from the genus Dirofilaria, including Dirofilaria immitis and Dirofilaria repens.

In a further preferred embodiment the invertebrate is selected from the genus Ancylostoma, including Ancylostoma braziliense, Ancylostoma caninum, Ancylostoma ceylanicum, Ancylostoma duodenale and Ancylostoma tubaeforme.

In a further preferred embodiment the invertebrate is selected from the genus Strongylus, including Strongylus asini, Strongylus edentatus, Strongylus equinus, Strongylus vulgaris.

In a further preferred embodiment the invertebrate is selected from the genus Trichuris, including Trichuris arvicolae, Trichuris leporis, Trichuris muris, Trichuris ovis, Trichuris skrjabini, Trichuris suis, Trichuris trichiura and Trichuris vulpis.

In a further preferred embodiment the invertebrate is selected from the genus Fasciola, including Fasciola gigantica and Fasciola hepatica.

In a further preferred embodiment the invertebrate is selected from the genus Heterophyes, including Heterophyes nocens

In a further preferred embodiment the invertebrate is selected from the genus Opisthorchis, including Opisthorchis felineus, Opisthorchis sinensis, Opisthorchis viverrini and Clonorchis sinensis.

In a further preferred embodiment the invertebrate is selected from the genus Taenia, including Taenia asiatica, Taenia crassiceps, Taenia hydatigena, Taenia martis, Taenia multiceps, Taenia ovis, Taenia parva, Taenia pisiformis, Taenia saginata, Taenia saginata asiatica, Taenia serialis, Taenia solium, Taenia taeniaeformis and enchinococcus.

In a further preferred embodiment the invertebrate is selected from the genus Diphyllobothrium, including Diphyllobothrium cordatum, Diphyllobothrium dendriticum, Diphyllobothrium ditremum, Diphyllobothrium lanceolatum, Diphyllobothrium latum, Diphyllobothrium nihonkaiense, Diphyllobothrium pacificum and Diphyllobothrium stemmacephalum.

The arthropod can be any organism classified in this taxonomical group. Preferably, the arthropod is selected from the group consisting of: Crustacea, Insecta and Arachnida.

Examples of preferred Insecta include, but are not limited to, members of the orders Coleoptera (e.g. Anobium, Ceutorhynchus, Rhynchophorus, Cospopolites, Lissorhoptrus, Meligethes, Hypothenemus, Hylesinus, Acalymma, Lema, Psylliodes, Leptinotarsa, Gonocephalum, Agriotes, Dermolepida, Heteronychus, Phaedon, Tribolium, Sitophilus, Diabrotica, Anthonomus or Anthrenus spp.), Lepidoptera(e.g. Ephestia, Mamestra, Earias, Pectinophora, Ostrinia, Trichoplusia, Pieris, Laphygma, Agrotis, Amathes, Wiseana, Tryporyza, Diatraea, Sporganothis, Cydia, Archips, Plutella, Chilo, Heliothis, Helicoverpa, Spodoptera or Tineola ssp), Diptera (e.g. Musca, Aedes, Anopheles, Culex, Glossina, Simulium, Stomoxys, Haematobia, Tabanus, Hydrotaea, Lucilia, Chrysomia, Callitroga, Dermatobia, Gasterophilus, Hypoderma, Hylemyia, Atherigona, Chlorops, Phytomyza, Ceratitis, Liriomyza, and Melophagus spp.), Phthiraptera, Hemiptera (e.g. Aphis, Bemisia, Phorodon, Aeneoplamia, Empoasca, Parkinsiella, Pyrilla, Aonidiella, Coccus, Pseudococcus, Helopeltis, Lygus, Dysdercus, Oxycarenus, Nezara, Aleurodes, Triatoma, Rhodnius, Psylla, Myzus, Megoura, Phylloxera, Adelyes, Niloparvata, Nephrotettix or Cimex spp.), Orthoptera (e.g. Locusta, Gryllus, Schistocerca or Acheta spp.), Dictyoptera (e.g. Blattella, Periplaneta or Blatta spp.), Hymenoptera (e.g. Athalia, Cephus, Atta, Lasius, Solenopsis or Monomorium spp.), Isoptera (e.g. Odontotermes and Reticulitermes spp.), Siphonaptera (e.g. Ctenocephalides or Pulex spp.), Thysanura (e.g. Lepisma spp.), Dermaptera (e.g. Forficula spp.) and Psocoptera (e.g. Peripsocus spp.) and Thysanoptera (e.g. Thrips tabaci).

Examples of preferred Arachnida include, but are not limited to, ticks, e.g. members of the genera Boophilus, Ornithodorus, Rhipicephalus, Amblyomma, Hyalomma, Ixodes, Haemaphysalis, Dermocentor and Anocentor, and mites and manges such as Acarus, Tetranychus, Psoroptes, Notoednes, Sarcoptes, Psorergates, Chorioptes, Demodex, Panonychus, Bryobia and Eriophyes spp.

Examples of preferred Crustaceans include, but are not limited to, crayfish, prawns, shrimps, lobsters and crabs.

Examples of Protozoa include, but are not limited to, Cryptosporidium sp., Giardia sp., Ameba (Entamoeeba) Toxoplasma, Isospora sp., Pneumocystiis sp.,

Dientamoeba sp., Chilomastix sp., Sarcocystis sp., Balantidium sp., Babesia sp., Retortamonas sp., Neopora sp., Trichomanas sp., Aegieria sp., Acanthamoeba sp., Entamoeb sp., Anaplasma sp. and Plasmodium sp.

Other, non-limiting, examples include Schistosomes (important in humans), Onchocerca (humans and animals), sandflies (as vector of leishmania), argas species (ticks), Eimeria, Trypanosomatids.

Non-limiting examples of compounds useful in the methods of the present invention comprise any one or more compound(s) selected from compounds: (19), (20), (7), (5), (4), (10c), (8), (11), (95), (16), (13), (68), (15), (18), (65), (123), (13a), (130), (95), (24), (65), (18), (65), (53), (71), (70), (10a), (10b), (23), (24), (13a), (129), (40a), (66), (67), (41), (42a), (57), (58), (102), (54), (53), (109), (103), (69), (72), (73), (117), (38), (46), (49), (26), (124), (31a), (112), (121), (113), (112), (23a), (22), (40), (33), (32), (24a), (39), (37), (50), (60), (47), (17a), (19a), (69), (71), (72) and/or (96).

### Preparation of Invertebrate Control Agents

General Procedure for the preparation of 7-Oxabicyclo(2.2.1)heptane-2,3-dicarboximide derivatives

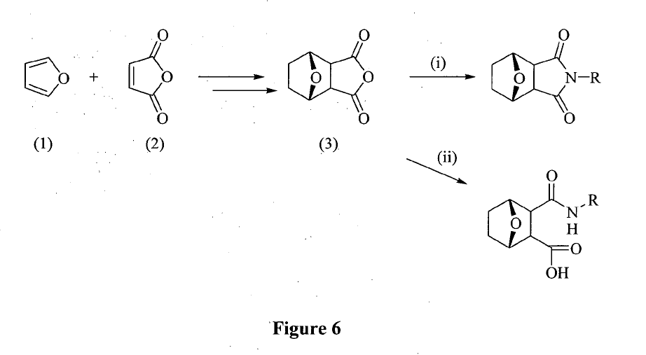

As shown in FIG. 6 (i), the general synthetic methodology involves reaction of furan (1) with maleic anhydride (2) to afford after dehydrogenation and purification 7-oxabicyclo(2.2.1)heptane-2,3-dicarboxylic anhydride (3). To a stirred solution of (3) with triethylamine in toluene, the required amine is added and the solution refluxed for 36 hours, cooled and diluted with EtOAc and washed with a NaHCO₃ solution to afford the crude 7-oxabicyclo(2.2.1)heptane-2,3-dicarboximide. After purification of the crude by either recrystallisation from EtOAc/Hexane or subjection to flash chromatography (−40% EtOAc/Hexane) the pure 7-oxabicyclo(2.2.1)heptane-2,3-dicarboximide analogue in 30-90% yield (depending on amine).

General Procedure for the preparation of 7-Oxabicyclo(2.2.1)heptane-2-carboxylic acid derivatives

As shown in FIG. 6 (ii), to a stirred solution of (3) in THF with, the required amine is added and the solution stirred at room temperature for 12 hours. After a standard workup, the crude 7-Oxabicyclo(2.2.1)heptane-2-carboxylic acid was purified by either recrystallisation or column chromatography to afford the pure 7-Oxabicyclo(2.2.1)heptane-2-carboxylic acid derivative.

General Procedure for the preparation of 7-Oxabicyclo(2.2.1)heptane-2-carboxylic epoxide derivatives

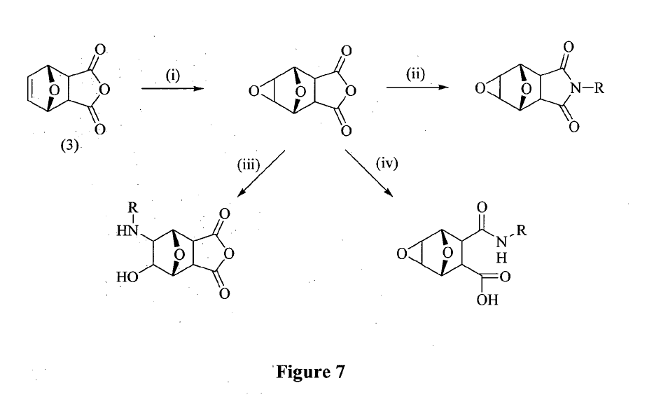

By a slight modification of the synthetic methodology employed to prepare the dicarboximide and carboxylic acid derivatives, the epoxide derivatives can be prepared. As shown in FIG. 7, to a stirred solution of 7-oxabicyclo(2.2.1)heptane-2,3-dicarboxylic anhydride an epoxidizing agent was added to yield the epoxide anhydride derivative. This was then reacted with the appropriate amine to produce the dicarboimide derivative as shown in FIG. 7 (ii) or the carboxylic acid derivative, FIG. 7(iv). Alternativly, the epoxide was ring opened by reaction with an amine via Figure (iii) to produce the substituded alcohol.

### Polypeptides

By “substantially purified polypeptide” we mean a polypeptide that has generally been separated from the lipids, nucleic acids, other polypeptides, and other contaminating molecules with which it is associated in its native state. With the exception of other proteins of the invention, it is preferred that the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. The terms “proteins” and “polypeptides” as used herein also include variants, mutants, modifications, analogous and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

As used herein a “biologically active” fragment is a portion of a polypeptide of the invention which maintains a defined activity of the full-length polypeptide, namely phosphatase activity. Biologically active fragments can be any size as long as they maintain the defined activity.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to, arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques include sub-cloning the polynucleotide into a suitable vector; transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they possess phosphatase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 2 under the heading of “exemplary substitutions”:

TABLE 4
Exemplary substitutions.

| Original | Exemplary |
|---|---|
| Residue | Substitutions |
| Ala (A) | val; leu; ile; gly; cys; ser; thr |
| Arg (R) | lys |
| Asn (N) | gln; his |
| Asp (D) | glu |
| Cys (C) | Ser; thr; ala; gly; val |
| Gln (Q) | asn; his |
| Glu (E) | asp |
| Gly (G) | pro; ala; ser; val; thr |
| His (H) | asn; gln |
| Ile (I) | leu; val; ala; met |
| Leu (L) | ile; val; met; ala; phe |
| Lys (K) | arg |
| Met (M) | leu; phe |
| Phe (F) | leu; val; ala |
| Pro (P) | gly |
| Ser (S) | thr; ala; gly; val; gln |
| Thr (T) | ser; gln; ala |
| Trp (W) | tyr |
| Tyr (Y) | trp; phe |
| Val (V) | ile; leu; met; phe; ala; ser; thr |

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Oligonucleotides and/or polynucleotides of the invention hybridize to a phosphatase gene of the present invention, or a region flanking said gene, under stringent conditions. The term “stringent hybridization conditions” and the like as used herein refers to parameters with which the art is familiar, including the variation of the hybridization temperature with length of an oligonucleotide. Nucleic acid hybridization parameters may be found in references which compile such methods, Sambrook, et al. (supra), and Ausubel, et al. (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2.xSSC, 0.01% BSA at 50° C. Alternatively, the nucleic acid and/or oligonucleotides (which may also be referred to as “primers” or “probes”) hybridize to the region of an invertebrate genome of interest, such as the genome of a nematode, under conditions used in nucleic acid amplification techniques such as PCR.

Oligonucleotides of the present invention can be RNA, DNA, or derivatives of either. Although the terms polynucleotide and oligonucleotide have overlapping meaning, oligonucleotides are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a polynucleotide or oligonucleotide are linked by phosphodiester bonds or analogs thereof to form oligonucleotides ranging in size from relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

The present invention includes oligonucleotides that can be used as, for example, probes to identify nucleic acid molecules, or primers to produce nucleic acid molecules. Oligonucleotides of the present invention used as a probe are typically conjugated with a detectable label such as a radioisotope, an enzyme, biotin, a fluorescent molecule or a chemiluminescent molecule.

### Antisense Polynucleotides

The term “antisense polynucleotide” shall be taken to mean a DNA or RNA, or combination thereof, molecule that is complementary to at least a portion of a specific mRNA molecule encoding a polypeptide of the invention and capable of interfering with a post-transcriptional event such as mRNA translation. The use of antisense methods is well known in the art (see for example, G. Hartmann and S. Endres, Manual of Antisense Methodology, Kluwer (1999)).

As used herein, the term “an antisense polynucleotide which hybridises under physiological conditions” means that the polynucleotide (which is fully or partially single stranded) is at least capable of forming a double stranded polynucleotide with mRNA encoding a protein, such as a protein comprising an amino acid sequence provided in SEQ ID NO:1, under normal conditions in a cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition. The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988, Perriman et al., 1992) and the hairpin ribozyme (Shippy et al., 1999).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for the ribozymes of this invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides of the invention should also be capable of hybridizing a target nucleic acid molecule (for example an mRNA encoding a polypeptide provided as SEQ ID NO:1, under “physiological conditions”, namely those conditions within a cell (especially conditions in an invertebrate cell)).

### RNA Interference

RNA interference (RNAi) is particularly useful for specifically inhibiting the production of a particular protein. Although not wishing to be limited by theory, Waterhouse et al. (1998) have provided a model for the mechanism by which dsRNA can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous animal system that destroys both the double stranded RNA and also the homologous RNA transcript from the target animal gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of an RNA polymerase II or RNA polymerase III promoter. Examples of the latter include tRNA or snRNA promoters.

Preferred small interfering RNA (‘siRNA”) molecules comprise a nucleotide sequence that is identical to about 19-21 contiguous nucleotides of the target mRNA. Preferably, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the organism in which it is to be introduced, e.g., as determined by standard BLAST search. An example of siRNA molecules that may be used to down-regulate the production of a polypeptide with phosphatase activity comprise a sequence selected from, but not limited to, the nucleotide sequences provided in SEQ ID NO: 21.

microRNA

MicroRNA regulation is a clearly specialized branch of the RNA silencing pathway that evolved towards gene regulation, diverging from conventional RNAi/post-transcriptional gene silencing (PTGS). MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RNA induced silencing complex (RISC)-like complexes containing a particular subset of Argonaute proteins that exert sequence-specific gene repression (see, for example, Millar and Waterhouse, 2005; Pasquinelli et al., 2005; Almeida and Allshire, 2005).

### Cosuppression

Another molecular biological approach that may be used is co-suppression. The mechanism of co-suppression is not well understood but is thought to involve PTGS and in that regard may be very similar to many examples of antisense suppression. It involves introducing an extra copy of a gene or a fragment thereof into an invertebrate in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target animal gene. Reference is made to WO 97/20936 and EP 0465572 for methods of implementing co-suppression approaches.

### Recombinant Vectors

One embodiment of the present invention includes a recombinant vector, which comprises at least one isolated polynucleotide molecule of the present invention, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises a polynucleotide molecule of the present invention operatively linked to an expression vector. The phrase operatively linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Particularly preferred expression vectors of the present invention can direct gene expression in animal cells. Vectors of the invention can also be used to produce the polypeptide in a cell-free expression system, such systems are well known in the art.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, plant or mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Particularly preferred transcription control sequences are promoters active in directing transcription in invertebrates, either constitutively or stage and/or tissue specific.

Recombinant molecules of the present invention may also (a) contain secretory signals (i.e., signal segment nucleic acid sequences) to enable an expressed polypeptide of the present invention to be secreted from the cell that produces the polypeptide and/or (b) contain fusion sequences which lead to the expression of nucleic acid molecules of the present invention as fusion proteins. Examples of suitable signal segments include any signal segment capable of directing the secretion of a polypeptide of the present invention. Preferred signal segments include, but are not limited to, tissue plasminogen activator (t-PA), interferon, interleukin, growth hormone, viral envelope glycoprotein signal segments, Nicotiana nectarin signal peptide (U.S. Pat. No. 5,939,288), tobacco extensin signal, the soy oleosin oil body binding protein signal, Arabidopsis thaliana vacuolar basic chitinase signal peptide, as well as native signal sequences of a polypeptide of the invention. In addition, a nucleic acid molecule of the present invention can be joined to a fusion segment that directs the encoded polypeptide to the proteosome, such as a ubiquitin fusion segment. Recombinant molecules may also include intervening and/or untranslated sequences surrounding and/or within the nucleic acid sequences of the present invention.

### Host Cells

Another embodiment of the present invention includes a recombinant cell comprising a host cell transformed with one or more recombinant molecules of the present invention, or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides of the present invention or can be capable of producing such polypeptides after being transformed with at least one polynucleotide molecule of the present invention. Host cells of the present invention can be any cell capable of producing at least one protein of the present invention, and include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells. Particularly preferred host cells are animal cells such as those available from Deutsche Sammlung von Mikroorganismen and Zellkulturen GmbH (German Collection of Microorganisms and Cell Cultures).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Target plants include, but are not limited to, the following: cereals (wheat, barley, rye, oats, rice, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers).

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

Constitutive plant promoters are well known. Further to previously mentioned promoters, some other suitable promoters include but are not limited to the nopaline synthase promoter, the octopine synthase promoter, CaMV 35S promoter, the ribulose-1,5-bisphosphate carboxylase promoter, Adhl-based pEmu, Actl, the SAM synthase promoter and Ubi promoters and the promoter of the chlorophyll a/b binding protein. Alternatively it may be desired to have the transgene(s) expressed in a regulated fashion. Regulated expression is possible by placing the coding sequence under the control of promoters that are tissue-specific, developmental-specific, or inducible. Several tissue-specific regulated genes and/or promoters have been reported in plants. These include genes encoding the seed storage proteins (such as napin; cruciferin, β-conglycinin, glycinin and phaseolin), zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development (such as Bce4). Particularly useful for seed-specific expression is the pea vicilin promoter. Other useful promoters for expression in mature leaves are those that are switched on at the onset of senescence, such as the SAG promoter from Arabidopsis). A class of fruit-specific promoters expressed at or during anthesis through fruit development, at least until the beginning of ripening, is discussed in U.S. Pat. No. 4,943,674. Other examples of tissue-specific promoters include those that direct expression in tubers (for example, patatin gene promoter), and in fiber cells (an example of a developmentally-regulated fiber cell protein is E6 fiber).

Other regulatory sequences such as terminator sequences and polyadenylation signals include any such sequence functioning as such in plants, the choice of which would be obvious to the skilled addressee. The termination region used in the expression cassette will be chosen primarily for convenience, since the termination regions appear to be relatively interchangeable. The termination region which is used may be native with the transcriptional initiation region, may be native with the polynucleotide sequence of interest, or may be derived from another source. The termination region may be naturally occurring, or wholly or partially synthetic. Convenient termination regions are available from the Ti-plasmid of A. tumefaciens, such as the octopine synthase and nopaline synthase termination regions or from the genes for β-phaseolin, the chemically, inducible lant gene, pIN.

Several techniques are available for the introduction of an expression construct containing a nucleic acid sequence encoding a polypeptide of interest into the target plants. Such techniques include but are not limited to transformation of protoplasts using the calcium/polyethylene glycol method, electroporation and microinjection or (coated), particle bombardment. In addition to these so-called direct DNA transformation methods, transformation systems involving vectors are widely available, such as viral and bacterial vectors (e.g. from the genus Agrobacterium). After selection and/or screening, the protoplasts, cells or plant parts that have been transformed can be regenerated into whole plants, using methods known in the art. The choice of the transformation and/or regeneration techniques is not critical for this invention.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Transgenic Hon-Human Animals

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Antibodies

The invention also provides monoclonal or polyclonal antibodies to polypeptides of the invention or fragments thereof. Thus, the present invention further provides a process for the production of monoclonal or polyclonal antibodies to polypeptides of the invention.

The term “binds specifically” refers to the ability of the antibody to bind to at least one polypeptide of the present invention but not other known phosphatase proteins.

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

Preferably, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect polypeptides of the invention.

### Methods of Use

The invertebrate controlling agents of the present invention can be formulated as dusts, water dispersions, emulsions, and solutions. They may comprise accessory agents such as dust carriers, solvents, emulsifiers, wetting and dispersing agents, stickers, deodorants and masking agents.

Dusts generally will contain low concentration, 0.1-20%, of the compounds, although ground preparations may be used and diluted. Carriers commonly include sulfur, silicon oxides, lime, gypsum, talc, pyrophyllite, bentonites, kaolins, attapulgite, and volcanic ash. Selection of the carrier can be made on the basis of compatibility with the desired pest control composition (including pH, moisture content, and stability), particle size, abrasiveness, absorbability, density, wettability, and cost. The agent of the invention alone or in combination and diluent is made by a variety of simple operations such as milling, solvent-impregnations, fusing and grinding. Particle sizes usually range from 0.5-4.0 microns in diameter.

Wettable powders can be prepared by blending the agents of the invention in high concentrations, usually from 15-95%, with a dust carrier such as bentonite which wets and suspends properly in water. 1 to 2% of a surface-active agent is usually added to improve the wetting and suspendibility of the powder.

The invertebrate controlling agent(s) can also be used in granules, which are pelleted mixtures of the agents, usually at 2.5-10%, and a dust carrier, e.g., adsorptive clay, bentonite or diatomaceous earth, and commonly within particle sizes of 250 to 590 microns. Granules can be prepared by impregnations of the carrier with a solution or slurry of the agents and can be used principally for mosquito larvae treatment or soil applications.

The agents can also be applied in the form of an emulsion, which comprises a solution of the agents in water immiscible organic solvents, commonly at 15-50%, with a few percent of surface active agent to promote emulsification, wetting, and spreading. The choice of solvent is predicated upon solubility, safety to plants and animals, volatility, flammability, compatibility, odour and cost. The most commonly used solvents are kerosene, xylenes, and related petroleum fractions, methylisobutylketone and amyl acetate. Water emulsion sprays from such emulsive concentrates can be used for plant protection and for household insect control.

The agents can also be mixed with baits, usually comprising 1-5% of agents with a carrier especially attractive to insects. Carriers include sugar for house flies, protein hydrolysate for fruit flies, bran for grasshoppers, and honey, chocolate or peanut butter for ants.

One embodiment of the present invention is a controlled release formulation that is capable of slowly releasing a composition of the present invention into/onto an animal, plant or the environment. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

In another embodiment of the present invention, the present invention is a sustained release formulation which is capable of releasing a composition of the present invention over an extended period.

A preferred controlled release formulation of the present invention is capable of releasing a composition of the present invention into the blood of an animal at a constant rate sufficient to attain effective dose levels of the composition to protect an animal from invertebrate infestation. The composition is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

Another preferred administration method of the present invention is via a topical administration of the invertebrate control agent(s) to the subject, preferably by application to the skin of the subject. For example, in the case of a cat or dog, the invertebrate control agent(s) may be applied to the skin for absorption or alternatively, the compositions and formulations of the present invention may be applied to the fur of the subject, for example a dog, for the control of ecto parasites.

The invertebrate controlling agent(s) of the present invention may be applied depending on the properties of the particular invertebrate controlling compound, the habits of the pest to be controlled and the site of the application to be made. It can be applied by spraying, dusting or fumigation.

Sprays are the most common means of application and generally will involve the use of water as the principal carrier, although volatile oils can also be used. The pest-control agents of the invention can be used in dilute sprays (e.g., 0.001-10%) or in concentrate sprays in which the composition is contained at 10-98%, and the amount of carrier to be applied is quite reduced. The use of concentrate and ultra low volume sprays will allow the use of atomizing nozzles producing droplets of 30 to 80 microns in diameter. Spraying can be carried out by airplane or helicopter.

Aerosols can also be used to apply the invertebrate controlling agent(s). These are particularly preferred as space sprays for application to enclosures, particularly against flying insects. Aerosols are applied by atomizing amounts of a liquefied gas dispersion or bomb but can be generated on a larger scale by rotary atomizers or twin fluid atomizers.

A simple means of invertebrate control agent(s) dispersal is by dusting. The invertebrate controlling agent(s) is/are applied by introducing a finely divided carrier with particles typically of 0.5-3 microns in diameter into a moving air stream.

Compositions of the present invention can be administered to any animal susceptible to invertebrate infestation (i.e., a host animal), including warm-blooded animals. Preferred animals to treat include mammals and birds, with cats, dogs, humans, cattle, chinchillas, ferrets, goats, mice, minks, rabbits, raccoons, rats, sheep, squirrels, swine, chickens, ostriches, quail and turkeys as well as other furry animals, pets, zoo animals, work animals and/or food animals, being more preferred. Particularly preferred animals to protect are sheep and cattle.

The optimal mode(s) of application will vary with the type of invertebrate pest and specific environmental conditions present at an infestation site. In some cases it is desirable to use a direct contact mode of application and, e.g., a spraying technique will be employed. Where pests are located in less accessible places such as in structural cracks in or behind structural gaps in a building, pavement, fixture, article of furniture, or in tree bark, either an injection or a pressurized spraying technique is typically preferred. A preferred direct mode of application for structure damaging pests comprises injecting the composition, e.g., into “galleries” within the structure where the pest colonies are located.

The concentration of the invertebrate control agent that will be required to produce effective compositions for the control of an arthropod pest will depend on the type of invertebrate and the formulation of the composition. The effective concentration of the invertebrate control agent within the composition can readily be determined experimentally, as will be understood by the skilled artisan. For example, the effective concentration of a virus can be readily determined using techniques known to the art.

Acceptable protocols to administer compositions of the present invention to animals in an effective manner include individual dose size, number of doses, frequency of dose administration, and mode of administration. Determination of such protocols can be accomplished by those skilled in the art. A suitable single dose is a dose that is capable of protecting an animal from invertebrate infestation when administered one or more times over a suitable time period. For example, a preferred single dose of a composition comprising a polypeptide, polynucleotide or invertebrate control agent of the present invention is from about 1 microgram to about 10 milligrams of the composition per kilogram body weight of the animal. Boosters can be administered from about 2 weeks to several years after the original administration. A preferred administration schedule is one in which from about 10 μg to about 1 mg of the composition per kg body weight of the animal is administered from about one to about two times over a time period of from about 2 weeks to about 12 months. Modes of administration can include, but are not limited to, subcutaneous, intradermal, intravenous, intranasal, oral, transdermal, intraocular and intramuscular routes.

### Agonists and Antagonists—Assays and Molecules

Polypeptides of the invention may be employed in a screening process for compounds which activate (agonists) or inhibit (antagonists) the phosphatase activity of the polypeptide.

Examples of potential antagonists include antibodies, oligosaccharides and derivatives thereof. A potential antagonist includes a small molecule which binds to the phosphatase, making it inaccessible to a substrate of the polypeptide. Examples of small molecules include, but are not limited to, small peptides or peptide-like molecules. The small molecules may mimic the structure of a substrate of the phosphatase.

In general, agonists or antagonists which can be used to regulate phosphatase activity are employed for purposes such as an agent for controlling invertebrate organism or organisms, typically those which are pests.

The invention also comprehends high-throughput screening (HTS) assays to identify compounds that interact with or inhibit the biological activity (i.e., affect enzymatic activity) of a phosphatase of the invention. HTS assays permit screening of large numbers of compounds in an efficient manner. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the polypeptide.

### Protein-Structure Based Design of Agonists and Antagonists

The three-dimensional structure of a crystal comprising a polypeptide having phosphatase activity (such as that provided as SEQ ID NO:1), or a fragment thereof, can be used to identify antagonists or agonists through the use of computer modeling using a docking program such as GRAM, DOCK, or AUTODOCK (Dunbrack et al., 1997). Computer programs can also be employed to estimate the attraction, repulsion, and steric hindrance of a candidate compound to the polypeptide. Generally the tighter the fit (e.g., the lower the steric hindrance, and/or the greater the attractive force) the more potent the potential agonist or antagonist will be since these properties are consistent with a tighter binding constant. Furthermore, the more specificity in the design of a potential agonist or antagonist the more likely that it will not interfere with other proteins.

Initially a potential compound could be obtained, for example, by screening a random peptide library produced by a recombinant bacteriophage, or a chemical library. A compound selected in this manner could be then be systematically modified by computer modeling programs until one or more promising potential compounds are identified.

Such computer modeling allows the selection of a finite number of rational chemical modifications, as opposed to the countless number of essentially random chemical modifications that could be made, and of which any one might lead to a useful agonist or antagonist. Each chemical modification requires additional chemical steps, which while being reasonable for the synthesis of a finite number of compounds, quickly becomes overwhelming if all possible modifications needed to be synthesized. Thus through the use of the three-dimensional structure and computer modeling, a large number of these compounds can be rapidly screened, and a few likely candidates can be determined without the laborious synthesis of untold numbers of compounds.

The prospective agonist or antagonist can be placed into the activity assay described herein to test its effect on the activity of a polypeptide having phosphatase activity.

For all of the screening assays described herein further refinements to the structure of the agonist or antagonist will generally be necessary and can be made by the successive iterations of any and/or all of the steps provided by the particular screening assay.

### NON-LIMITING EXAMPLES

### Example 1

### Cloning of a Phosphatase Gene from Trichostronevlus vitrinus

### Materials and Methods

### Parasites

Lambs (males; 8-12 weeks of age), maintained under helminth-free conditions, were infected intra-ruminally with 30,000 infective third-stage larvae (L3) of T. vitrinus. The patency of the infection (˜24 days after inoculation) was established via the detection of strongylid eggs in the faeces using the McMaster flotation method (MAFF, 1977). First stage (L1), second stage (L2) and L3 were collected from faecal cultures after incubation for 1, 3 or 7 days, respectively at 28° C. in an oxygen-rich, humid environment and were purified by sedimentation, followed by migration through a sieve (20 μm mesh size) for 16 h. For the collection of fourth-stage larvae (L4) and adults, lambs were euthanased with an overdose of pentobarbitone sodium (Virbac) administered intravenously 8 days (L4) and 30 days (adults) after inoculation with L3s. Worms were removed (using a fine forceps) from the chyme of the first 4 m of the small intestine, washed extensively in phosphate-buffered saline (PBS), males and females (adults) separated prior to snap freezing in liquid nitrogen and subsequent storage at −70° C. The specific identity of the parasite material was confirmed by PCR amplification of the internal transcribed spacer 2 (ITS-2) (as described by Hoste et al., 1995) and automated sequencing. The sequences determined were required to be identical to the ITS-2 sequence with GenBank accession number X78064 (Hoste et al., 1995).

### Isolation, Purification, Treatment and Storage of Nucleic Acids

Total genomic DNA was extracted from ±0.5 g of single sex (male or female) adult worms using a small-scale sodium-dodecyl-sulphate/proteinase K extraction procedure (Gasser et al., 1993), followed by mini-column (Wizard™ Clean-Up, Promega) purification. Total RNA was extracted separately from different developmental stages (L1, L2, L3, L4 or adults) or sexes of T. vitrinus (homogenized in liquid nitrogen using mortar and pestle) using the Tripure™ isolation reagent (Roche Molecular Biochemicals). RNA yields were estimated spectrophotometrically, and the integrity of RNA was confirmed via the detection of discrete 18S and 28S rRNA bands on ethidium bromide-stained gels. Each RNA sample (˜10 μg) was treated with 2 U of DNase I (Promega) and incubated at 37° C. for 30 min prior to heat denaturation of the enzyme (75° C. for 5 min). Both DNA and RNA were stored at −70° C.

Isolation of Full-Length cDNA of Tv-stp1 from T. vitrinus

A library was constructed from adult male T. vitrinus total RNA using the SMART™ PCR cDNA library construction kit (Clontech), as described in the manufacturer's instructions with the following modifications: (i) 19 cycles were determined to be optimal for the amplification of cDNA by long distance PCR, following first strand synthesis; (ii) column chromatography of the PCR products prior to digestion with the endonuclease SfI was replaced by purification using the Wizard® PCR Preps DNA purification system (Promega) to minimise cDNA loss. For library screening, 25,000 plaque forming units (pfu) were plated and lifted on to positively-charged nylon membranes (Roche Molecular Biochemicals). The membranes were pre-hybridised at 55° C. for 1 h in pre-hybridised buffer [0.25 M NaHPO₄, pH 6.5, 1 mM EDTA, 7% (w/v) sodium dodecyl-sulphate (SDS) and 100 μg/ml sonicated herring sperm DNA (Roche Molecular Biochemicals)]. The denatured probe, labelled by random priming with [α-³²P]dCTP (GeneWorks), was added to the pre-hybridisation solution and incubated overnight at 55° C. The filters were washed 3 times in 2×SSC (0.3 M NaCl, 30 mM Na citrate, pH 7.0), 0.1% SDS at 60° C. for 20 min and then subjected to autoradiography for 16-24 h.

Positively-hybridizing plaques were picked, the phage was eluted into 500 μl of SM buffer and re-screened by hybridization to ensure that they were clonal (Sambrook et al. 1989). A clone (Tv-1) with the longest insert (585 bp) was isolated and sequenced. Using gene-specific primers STRF2 (5′-CGAGGTGCCTCGTATGGTTTTGGA-3′) (SEQ ID NO:15) and STRR2 (5′-GGCTGAGAAAGAAACATTTCGCGTGCT-3′) (SEQ ID NO:16) designed to this sequence, two partially overlapping cDNA fragments were generated from total RNA from adult male worms using 5′ and 3′-rapid amplification of cDNA ends (RACE) (SMARTT″ RACE cDNA Amplification Kit, BD Biosciences). The cDNAs were ligated into the pGEM-T Easy vector (Promega), and recombinant plasmids were transformed to competent Escherichia coli (strain JM109) (10⁸ colony forming units/μg) via heat shock and grown overnight at 37° C. on Luria Bertani (LB) plates containing 10 mg/ml ampicillin, 0.5 mM isopropyl-β-D-thiogalactopyranoside (IPTG) and 80 μg/ml X-gal (5-bromo-4-chloro-3-indolyl-β-galactosidase. Plasmid DNA was isolated from recombinant clones and column-purified (Wizard, Promega) from overnight cultures and sequenced in both directions using vector primers (M13 and SP6), employing Big Dye Terminator v.3.1 chemistry in an automated ABI-PRISM sequencer (Applied Biosystems). Based on the resultant sequences, selected oligonucleotide primers were designed to amplify the full-length Tv-stp1 gene from T. vitrinus adult male cDNA, which was subsequently cloned and sequenced.

### Bioinformatic and Phylogenetic Analyses

The full-length cDNA sequence of Tv-stp1 was conceptually translated (six different frames) into amino acid sequences using the BCM Search Launcher (available at http://searchlauncher.bcm.tmc.edu/seq-util/Options/sixframe.html) and aligned using the ClustalW program (Thompson et al. 1994; available at http://www.ebi.ac.uk/clustalw/index.html). Pairwise comparisons at the amino acid level were achieved using EMBOSS: needle (available at http://www.ebi.ac.uk/emboss/align/via the European Molecular Biology Laboratory, EMBL). Sequences were compared with those available in public non-redundant databases using BLASTn and BLASTx algorithms available at the National Centre for Biotechnology Information databases (http://www.ncbi.nlm.nih.gov/blast/), and the parasite genome WU-BLAST2 Nematoda database (from the European Bioinformatics Institute, http://www.ebi.ac.uk/blast2/parasites.html). The alignment of predicted protein sequences was carried out using the Clustal W program (Thompson et al., 1994).

Phylogenetic analyses of inferred amino acid sequence data were conducted using the program PAUP*4.0b10, as described previously (Hu et al., 2005). In brief, the neighbor-joining method was used to construct trees from distance data. The maximum parsimony method, based on character state analysis, was also used. Characters were treated as unordered and were equally weighted; alignment gaps were treated as ‘missing’ in all analysis. Exhaustive searches with TBR branch-swapping were used to infer the shortest trees. The length, consistency index, excluding uninformative characters, and the retention indices of each most parsimonious tree were recorded. Bootstrap analyses (1000 replicates) were conducted using heuristic searches and TBR branch-swapping, with the MulTrees option, to determine the relative support for clades in the consensus trees.

### Determination of the Full-Length Genomic Sequence of Tv-stp1 and its Structure

The entire Tv-stp1 gene was amplified by long-PCR (Expand™ 20 kbplus kit, Roche) from ˜100 ng of total genomic DNA purified from pooled T. vitrinus, employing primers 5′-STALLF3 (5′-ATGGACACTACTCAATTGATCACTAAC-3′) (SEQ ID NO:17) and STALLR3′ (5′-TCATTGACAAGGAGGCGCTG-′3) (SEQ ID NO:18) located at the 5′- and 3′-terminal regions of the Tv-stp1 cDNA, respectively. The cycling conditions in a 2400 thermocycler (ABI) were: 92° C., 2 min (initial denaturation); then 92° C., 10 s (denaturation); 60° C., 30 s (annealing); 68° C., 10 min (extension) for 10 cycles, followed by 92° C., 10 s; 60° C., 30 s; 68° C., 10 min for 20 cycles, with a cycle elongation of 10 s for each cycle, and a final extension at 68° C. for 7 min. Each PCR reaction yielded a single amplicon, detected in 1% (w/v) agarose gel upon ethidium-bromide staining. An abundant amplicon of ˜5.2 kb was excised from the gel, purified over a mini-spin column (PCR-Prep™, Promega), cloned into the plasmid vector pGEM-T-Easy and then used as a templates for automated sequencing (as described above), employing a panel of 11 different primers using a “walking strategy”. Sequences obtained were assembled using the CAP program (Huang et al., 1992) at the Resources Centre INFOBIOGEN (http://www.infobiogen.fr/services/analyseq/cgi-bin/cap_in.pl). The genomic sequence of Tv-stp1 was compared with those of selected (most closely related) C. elegans serine/threonine phosphatase genes (accession numberes: Z82263, AF016433, U88169, U88178, U00063, Z73974) and of Od-mpp1 from Oesophagostomum dentatum (accession number AF496634) obtained from NCBI databases (http://www.ncbi.nlm.nih.gov/entrez/query.fcgi?db=Nucleotide&itool=toolbar) or WormBase (http://www.wormbase.org/). The exon/intron boundaries of Tv-stp1 and related full-length genes were inferred based on the alignment of the Tv-stp1 cDNA with genomic DNA sequences, following the AG-GT rule.

### Analysis of Tv-stp1 Transcripts by Reverse Transcription PCR (RT-PCR)

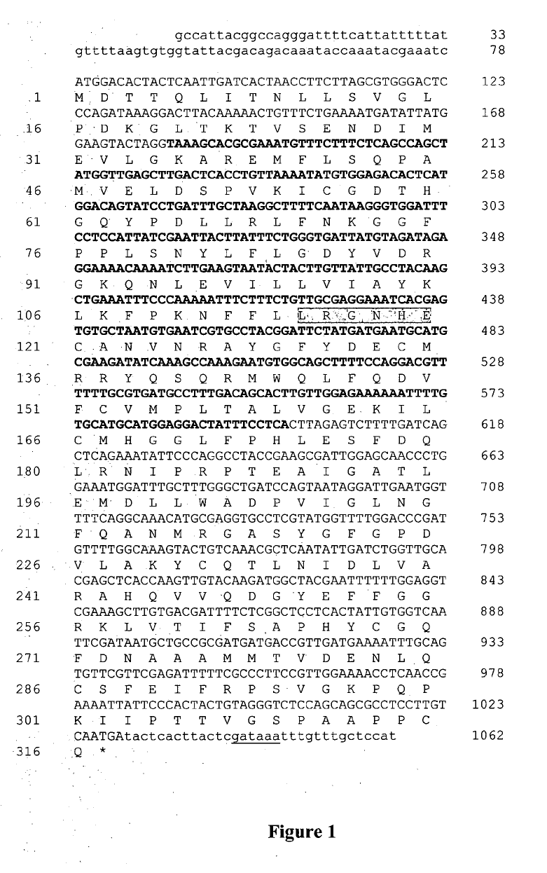

First-strand cDNA was synthesized from DNase I-treated total RNA (˜3 μg) from different developmental stages and both sexes of T. vitrinus by random priming using Superscript II reverse transcriptase (cat. no. 18064-022, Invitrogen), as recommended by the manufacturer. Thereafter, 10% of the cDNA synthesized was subjected to the PCR (50 μl reaction volume) in a 2400 thermocycler (ABI, Australia) under the following conditions: 1 cycle of 94° C. for 5 min, followed by 30 cycles of 92° C. for 30 s, 60° C. for 30 s, 68° C. for 45 s, followed by 1 cycle of 68° C. for 7 min. The transcript representing Tv-stp1 (951 bp; FIG. 1) was amplified using primers STALLF3 (5′-ATGGACACTACTCAATTGATCACTAAC-3′) (SEQ ID NO:17) and STALLR3 (5′-TCATTGACAAGGAGGCGCTG-3′) (SEQ ID NO:18). Using these primers, no product of >5 kb (cf. section 2.5) was amplified from any of the cDNA samples, inferring the absence of genomic DNA from them. The primers 28S1/F (5′-GCATAAGCTCTCGCGTTACC-3′; forward) (SEQ ID NO:19) and 28S3/R (5′-GAGAGGGACAGCAGGTTCAC-3′; reverse) (SEQ ID NO:20) were used in the PCR (positive control) for the amplification of a short region (187 bp) from the large subunit of nuclear ribosomal RNA. No DNA control samples were included in each PCR run. Following the PCR, 10% of each amplicon were loaded and resolved in a 2% w/v ethidium bromide-stained agarose gel and photographed upon transillumination. The specificity and identity of individual amplicons were confirmed by direct sequencing using the same primers (individually) as employed for their amplification.

### Results

Characterisation of the Tv-stp1 cDNA and Comparison with Related Molecules

The full-length cDNA (designated Tv-stp1) isolated was 951 by (FIG. 1). This cDNA contained a 5′-UTR of 78 by and a 3′-UTR of 33 by which included a putative polyadenylation signal (GATAAA), commencing 14 nucleotides 5′ to the TGA (stop codon) (FIG. 1). The uninterrupted ORF of 951 nucleotides encoded a predicted protein of 316 amino acids, which contained a specific serine/threonine protein phosphatase pattern [LIVMN]-[KR]-G-N-H-E; the Prosite motif PS00125 was consistent with the gene C47A4.3 from C. elegans (FIG. 1).

Comparison with sequences in non-redundant databases by BLASTx analysis showed that the predicted protein representing Tv-stp 1 had homologies/identities/similarities to sequences from a range of organisms, including other nematodes, protists, vertebrates and plants (see Table 3). The greatest similarity/homology (E-values: 2e-100 to 4e-104) was to amino acid sequences predicted for the full-length C. elegans genes C47A4.3, C09H5.7, T03F1.5 and WO9C3.6, F56C9.1 and F29F11.6 and from Odmpp1 (accession no. AF496635; Boag et al., 2003) from the parasitic nematode Oesophagostomum dentatum, all of which represent the PP1 class of PPs (Barford et al., 1998). The highest BLASTx score (380) was to the yeast glc seven-like phosphatases protein 3 gene (gsp-3; C47A4.3) from C. elegans (see Table 3).

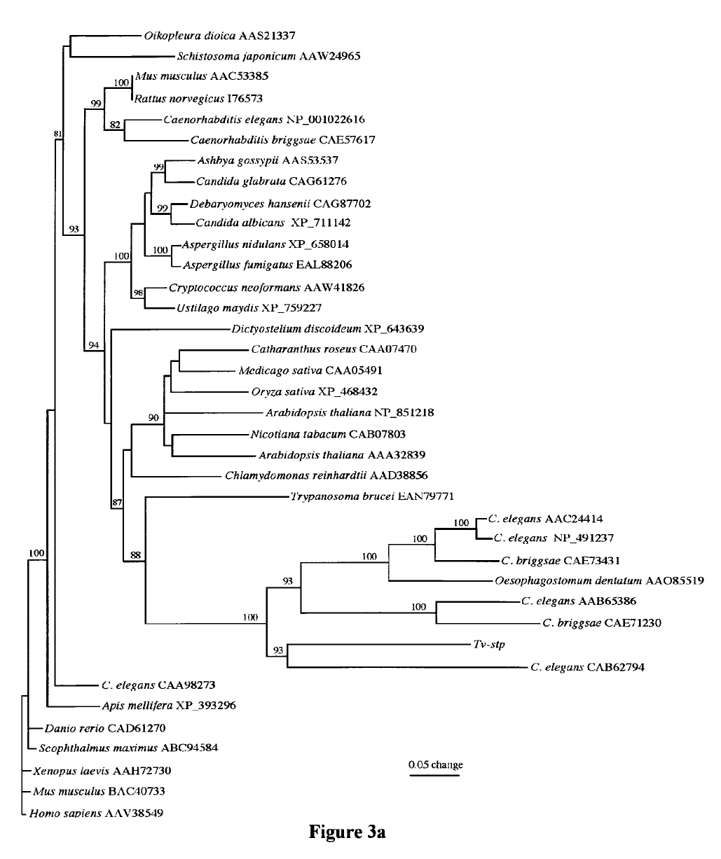

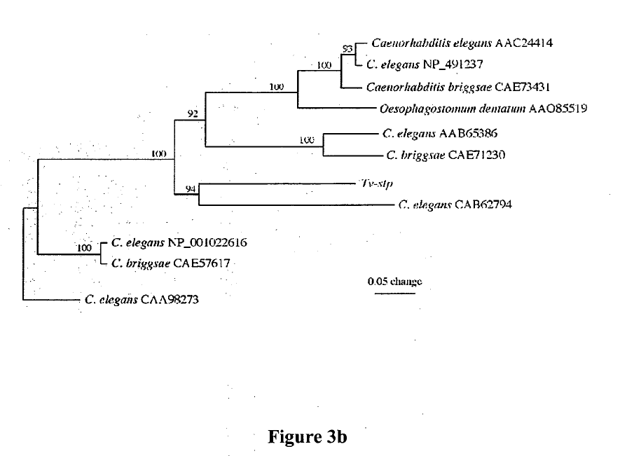

TABLE 3
Selected serine/threonine phosphatase amino acid sequences
(and relevant information from BLASTx analysis) used for the analysis
of phylogenetic relationship to Tv-STP1 from Trichostrongylus vitrinus (see FIG. 3)

| Group | Species | Accession no. | Gene locus | Gene name | Score | Expect | Identity (%) |
|---|---|---|---|---|---|---|---|
| Nematode | Caenorhabditis elegans | CAB62794 | C47A4.3 | Not available | 370 | 3e−101 | 60 |
| | C. elegans | AAB65386 | C09H5.7 | Not available | 374 | 4e−102 | 57 |
| | C. elegans | NP_491237 | T03F1.5 | gsp-4 | 378 | 2e−103 | 57 |
| | C. elegans | AAC24414 | W09C3.6 | gsp-3 | 380 | 4e−104 | 57 |
| | C. elegans | NP_001022616 | F56C9.1 | gsp-2 | 362 | 1e−98 | 56 |
| | C. elegans | CAA98273 | F29F11.6 | gsp-1 | 365 | 2e−99 | 56 |
| | C. briggsae | CAE73431 | CBG20874 | Not available | 373 | 5e−102 | 57 |
| | C. briggsae | CAE71230 | CBG18099 | Not available | 373 | 6e−102 | 58 |
| | C. briggsae | CAE57617 | CBG00598 | Not available | 362 | 1e−98 | 56 |
| | Oesophagostomum dentatum | AAO85519 | Od-mpp1 | Od-mpp1 | 374 | 4e−102 | 57 |
| Platyhelminth | Schistosoma japonicum | AAW24965 | AY813233 | Not available | 360 | 6e−98 | 57 |
| Insect | Apis mellifera | XP_393296 | XM_393296 | L0C409804 | 359 | 7e−98 | 53 |
| Protozoa | Trypanosoma brucei | EAN79771 | Tb11.01.0450 | Not available | 365 | 1e−99 | 55 |
| | Dictyostelium discoideum | XP_643639 | DOBO185058 | PPPB | 358 | 2e−97 | 55 |
| Fungus | Ashbya gossypii | AAS53537 | AFR166C | Not available | 363 | 4e−99 | 55 |
| | Aspergillus nidulans | XP_658014 | AN0410.2 | Not available | 361 | 2e−98 | 54 |
| | A. fumigatus | EAL88206 | Afulg04950 | Not available | 358 | 2e−97 | 54 |
| | Candida albicans | XP_711142 | CaO19.6285 | glc-7 | 359 | 7e−98 | 56 |
| | C. glabrata | CAG61276 | CAGLOK02079g | Not available | 358 | 2e−97 | 56 |
| | Cryptococcus neoformans | AAW41826 | CNB02030 | Not available | 359 | 7e−98 | 55 |
| Yeast | Debaryomyces hansenii | CAG87702 | DEHAOE04400g | Not available | 362 | 1e−98 | 54 |
| | Ustilago maydis | XP_759227 | UMO3080 | Not available | 358 | 2e−97 | 53 |
| Alga | Chlamydomonas reinhardtii | AAD38856 | NA | Not available | 359 | 1e−97 | 57 |
| Plant | Catharanthus roseus | CAA07470 | CRO7332 | Not available | 363 | 7e−99 | 59 |
| | Medicago sativa | CAA05491 | MSAJ2485 | PP1-beta | 363 | 7e−99 | 58 |
| | Arabidopsis thaliana | AAA32839 | ATHPPHAC | TOPP2 | 363 | 7e−99 | 58 |
| | A. thaliana | NP_851218 | AT5G59160 | TOPP2 | 360 | 4e−98 | 57 |
| | Oryza sativa | XP_468432 | XM_468432 | OJ1202_E07.27 | 360 | 3e−98 | 57 |
| | Nicotiana tabacum | CAB07803 | NTNPP1 | npp-1 | 358 | 1e−97 | 60 |
| Chordate | Oikopleura dioica | AAS21337 | OO2-15 | gsp-1 | 360 | 4e−98 | 54 |
| Fish | Scophthalmus maximus | ABC94584 | DQ364569 | PP1cb | 360 | 3e−98 | 54 |
| | Danio rerio | CAD61270 | 2C214P16.4-001 | Not available | 360 | 6e−98 | 54 |
| Amphibian | Xenopus laevis | AAH72730 | BC072730 | MGC79074 | 358 | 1e−97 | 54 |
| Mammal | Mus musculus | BAC40733 | NA | Not available | 360 | 3e−98 | 54 |
| | M. musculus | AAC53385 | AH006785S1-S6 | PP1cgamma | 359 | 7e−98 | 55 |
| | Rattus norvegicus | I76573 | NA | Not available | 359 | 7e−98 | 55 |
| | Homo sapiens | AAV38549 | BTO19744 | Not available | 358 | 1e−97 | 54 |

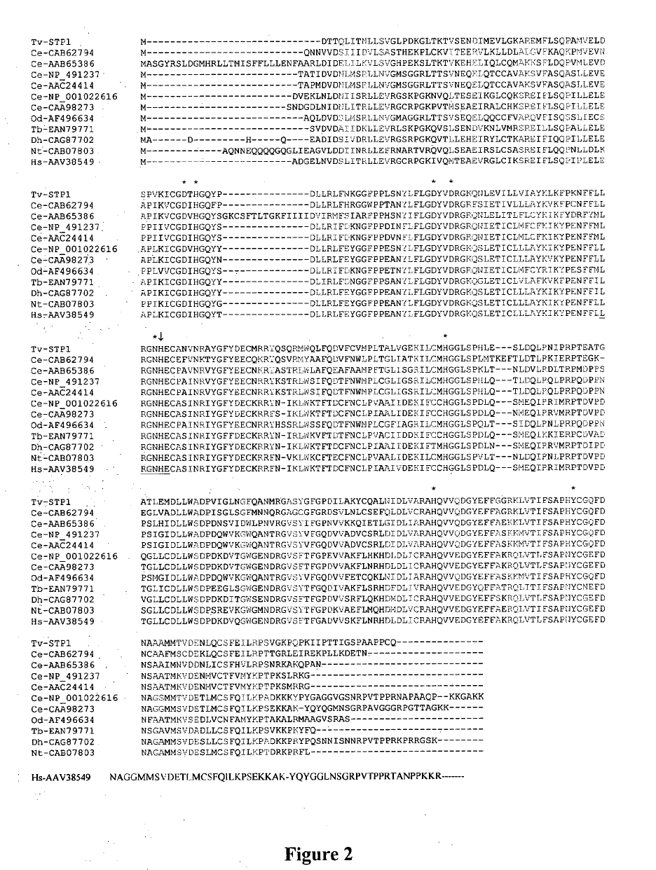

Comparison of the predicted amino acid sequence for TV-STP 1 with that of gsp-3 revealed 57% identity (FIG. 2). The second most similar protein predicted was from another C. elegans gene (C09H5.7, accession no. AAB65386.1), which contained insertions of 30 amino acid residues at the N-terminus (positions 1-32) and of 15 residues (positions 63-76) in relation to Tv-STP1 (FIG. 2). An alignment of the predicted serine phosphatase sequences from nematodes with selected representatives from different eukaryotic groups (FIG. 2) also demonstrated sequence identity (52.7-56.2%) with plant, animal and yeast proteins. Sequence similarity was most pronounced in the central region of the protein, where the catalytic activity of the PPs is predicted to be modulated by eight conserved residues (Asp 61, His 63, Asp 92, Asp 95, Asn 121, His 171, His 246 and Tyr 270; FIGS. 1 and 2), known to coordinate the binding of two metal ions (Mn²⁺and Fe²⁺) in a range of species (Barford et al., 1998). A seventh residue (His 122) acts as a proton donor to catalyse the cleavage of the phosphate group from phospho-serine or -threonine (Barford et al., 1998). The alignment also shows that the N- and C-terminal regions of the proteins are more variable than the catalytic part. These regions are suggested to interact with proteins which regulate the phosphatase activity (Egloff et al., 1997).

Phylogenetic Relationship of the Predicted Protein Tv-STP1 with Selected Molecules

The amino acid sequences predicted for selected molecules (n=37) (with an e-value ≧1e-97; score 358) together with that of Tv-stp1 were used to conduct phylogenetic analyses to establish which molecule(s) showed the closest relationship to TV-STP1 (FIG. 3A). There was concordance in the topology of the NJ and PAUP trees in that selected serine-threonine phosphatases from C. elegans, considered to represent gsp-3 or gsp-4, and from O. dentatum OD-MPP-1 grouped with TV-STP1 from T. vitrinus (with 100% bootstrap support) to the exclusion of those from other organisms (including protists, vertebrates and plants) and also to the exclusion of GSP-1 (F29F11.6; accession no. CAA98273.1) and GSP-2 (F56C9.1; accession no. NP_{—}001022616.1) from C. elegans (FIG. 3A). Consistent with the amino acid alignment presented (FIG. 2) and the analyses of selected nematode sequences (n=11) (using both algorithms; FIG. 3B), TV-STP1 had the closest relationship to GSP-3 (316 aa) predicted from C. elegans gene C47A4.3 (93% bootstrap support for NJ and 69% for PAUP), followed by C. elegans C09H5.7, O. dentatum Odmpp1 and C. elegans T03F1.5 and WO9C3.6 (91-100% bootstrap support for both methods).

### Sequence Analysis of Genomic Clones Representing Tv-stp1 and Genomic Organization

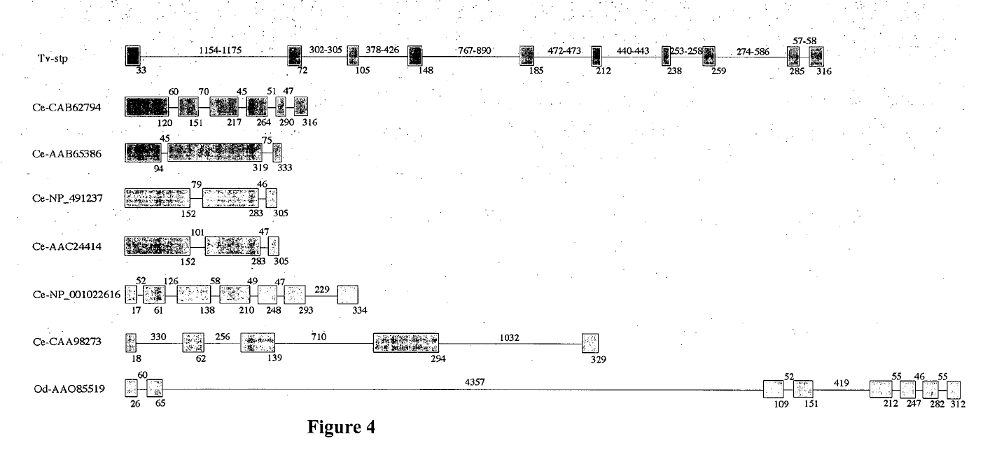

After the cloning of a long-range PCR product of ˜5.2 kb amplified from genomic DNA from T. vitrinus, four clones (nos. 5, 7, 10, 12) were isolated and fully sequenced. The four sequences were determined to be 5041 (clone no. 12), 5184 (clone no. 5), 5353 (clone no. 10) and 5362 (clone no. 7) nucleotides in length. The four genomic sequences were aligned (in the exon regions) with the Tv-stp1 cDNA sequence and the intron-exon boundaries determined. Ten exons and nine introns were defined (FIG. 4). For individual exons, nucleotide variation of 0-5.2% was recorded (upon pairwise comparison) among all four clones; this variation was represented by transitions (4 A<->G and 10 C<->T) and transversions (2 G<-T and 2 A<->C), 16 of which were silent (i.e. did not result in a change in the amino acid predicted; cf. FIG. 1). A substitution caused an amino acid change from Asn (AAT) to Ser (AGT) in exon 3 and another caused a change from Phe (TTC) to Ser (TCC) in exon 4. Intron sizes ranged from 58-1175 nucleotides in length, of which intron 1 was the longest and intron 9 the shortest. For individual introns, length/sequence variation of 1-312 nucleotides was recorded (upon pairwise comparison) among all four genomic sequences (cf. FIG. 4; introns 1-9). The greatest length variation (>100 nucleotides) was recorded among the sequences of introns, 4 and 8; length variation in the other 7 introns was <50 nucleotides (FIG. 4).

Comparison of the Genomic Organization of Tv-stp1 with those of Related Molecules

Based on the phylogenetic relationships (FIGS. 3A and 3B), the genomic structure of Tv-stp1 was compared with C. elegans genes C47A4.3, C09H5.7, T03F1.5, WO9C3.6, F56C91.2, F29F 11.6 encoding serine threonine phosphatases (accession nos. CAB62794.1, AAB65386.1, AAB42233.1, AAC24414.1, NP_{—}001022616.1 and CAA98273.1, respectively) and the genomic sequence representing O. dentatum Od-mpp1 (accession no. AF496634; Boag et al., 2003) (FIG. 4). Even though C. elegans C47A4.3 was considered to be closest genetically to Tv-stp1.g (FIGS. 3A and 3B), the structure of the latter was more complex (FIG. 4). Comparison of the structures revealed substantial variation in the lengths and numbers of the exons and introns. The C. elegans genes (1222-1603 bp) were usually considerably shorter than those from the parasitic nematodes T. vitrinus (5041-5362 bp) and O. dentatum (10271 bp). The C. elegans genes included had 3-7 exons (2-6 introns) compared with up to 10 for the parasitic nematodes (FIG. 4).

Transcriptional Analysis in Different Developmental Stages and After RNAi in Adult Males of T. vitrinus by Reverse Transcription PCR (RT-PCR)

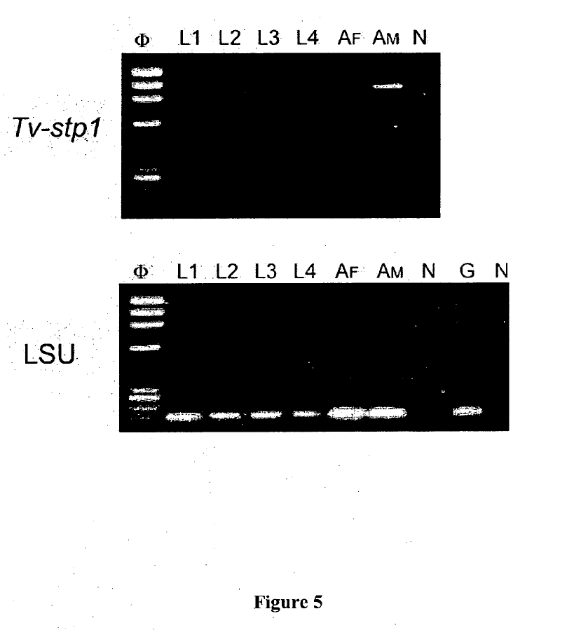

Based on results from a previous microarray analysis (Nisbet and Gasser, 2004), Tv-stp1 was shown to be enriched in adult males of T. vitrinus. The results of RT-PCR analyses (FIG. 5) showed that Tv-stp1 was transcribed in adult males of T. vitrinus. In the samples that were tested, no transcription was detectable in the adult female of T. vitrinus or in any of the larval stages included (L1-L4; mixed sexes) in the analysis under the conditions employed.

### Discussion

Tv-stp1, encoding a serine/threonine phosphatase, has been characterized. This gene and its inferred protein have identity/similarity to a number of molecules from a range of organisms, including nematodes. Interestingly, in spite of some conservation among Tv-STP1, Od-MPP1 and C. elegans PPs (particularly those predicted from C47A4.3 and C09H5.7), there are significant differences among these molecules in their genomic structure, particularly in relation to number and size of exons/introns. For example, the sizes of introns for the C. elegans genes are relatively typical and vary from 45-229 bp, which contrasts the situation for the Tv-stp1 gene, being significantly larger and more akin to the full-length gene Od-mpp1 in terms of exon/intron numbers and size (see FIG. 4). The intron sizes were also more variable (ranging from 57-1175 bp) than those from the C. elegans homologues (FIG. 4).

Testis-specific expression of both protein phosphatases and kinases has been reported for both invertebrates and vertebrates (Smith et al., 1996; Armstrong et al., 1998; Herrmann et al., 1998). In mice, the PP TMDP appears to be involved in the regulation of meiosis and/or differentiation of testicular germ cells (Nakamura et al., 1999). Also, in primates, some PPs are involved in the regulation of sperm motility (Smith et al., 1996). While some biological pathways involving phosphatases may be distinctly different between vertebrates and invertebrates (e.g., nematode sperm do not contain flagella), the pathways of spermatogenesis rely upon the regulation of protein activity via the actions of phosphatases and kinases.

Tv-stp1 from T. vitrinus, Od-mpp1 from O. dentatum and PPs from C. elegans do not contain nuclear-targeting signals or secretion signals, suggesting that they are not localized to the nucleus (and thus do not interact directly with transcription factors) or are secreted from the cell. This information suggests that these proteins are present in the cytoplasm, where they may be involved in signal transduction or the regulation of protein activity.

Following the cloning of a phosphatase gene from T. vitrinus, the present inventors screened known phosphatase inhibitors, and molecules related thereto, for their ability to control invertebrates.

### Example 2

### Preparation of 7-Oxabicyclo(2.2.1)heptane-2,3-dicarboximide derivatives

### General Synthetic Scheme for Dicarboxide Derivatives

Amine (1 eq, 2.97 mmol) was added to a magnetically stirred solution of 7-oxabicyclo(2.2.1)heptane-2,3-dicarboxylic anhydride (1.0 g, 2.97 mmol) and triethylamine (1.5 mL) in toluene (15 mL). This solution was refluxed for 36 h before being cooled, diluted with EtOAc (45 mL), washed with NaHCO₃ (2×5 mL, sat solution), filtered and concentrated under reduced pressure. The resulting crude 7-oxabicyclo(2.2.1)heptane-2,3-dicarboximide was either recrystallised from EtOAc/Hexane or subjected to flash chromatography (˜40% EtOAc/Hexane) to afford the pure 7-oxabicyclo(2.2.1)heptane-2,3-dicarboximide analogue in 30-90% yield (depending on amine).

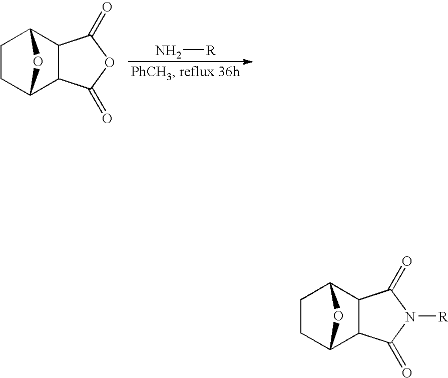

### N-propyl-7-Oxabicyclo(2.2.1)heptane-2,3-dicarboximide (4)

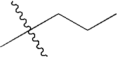

1-Aminopropane (0.176 g, 2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL), washed with NaHCO₃ (2×5 mL, sat solution), filtered and concentrated under reduced pressure. The resulting crude was recrystallised EtOAc/Hexane to afford the pure white solid N-propyl-7-Oxabicyclo(2.2.1)heptane-2,3-dicarboximide in a 80% yield. Mp: 79-80° C.; ¹H NMR (CDCl₃): 0.87 (t, J=7.3 Hz, 3H, CH₃), 1.55-1.61 (m, 4H, 2×CH₂), 1.83-1.86 (m, 2H, CH₂), 2.84 (s, 2H, 2×CH), 3.44 (t, J=7.3 Hz, CH₂), 4.86 (brs, 2H, CH); ¹³C NMR (CDCl₃): 10.5 (CH₃), 20.3 (CH₂), 28.0 (2×CH₂), 40.0 (CH₂), 49.3 (CH), 78.4 (CH₂), 176.6 (2×C═O).

### N-butyl-7-Oxabicyclo(2.2.1)heptane-2,3-dicarboximide (5)

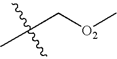

Butyl amine (0.217 g, 2.97 mmol) was added drop wise to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was recrystallised with EtOAc/Hexane to afford the pure N-butyl-7-Oxabicyclo(2.2.1)heptane-2,3-dicarboximide in a 93% yield. Mp: 82-83° C.; ¹H NMR (CDCl₃): 0.90 (t, J=7.2 Hz, 3H, CH₃), 1.28 (sep, J=7.8 Hz, 2H, CH₂), 1.49-1.59 (m, 4H, 2×CH₂), 1.82-1.85 (m, 2H, CH₂), 2.83 (s, 2H, 2×CH), 3.45 (t, J=7.3 Hz, 2H, CH₂), 4.85 (q, J=0.7 Hz, 2H, 2×CH); ¹³C NMR (CDCl₃): 12.9 (CH₃), 19.3 (CH₂), 28.0 (2×CH₂), 29.0 (CH₂), 38.2 (CH₂), 49.3 (2×CH) 78.4 (CH₂), 176.6 (2×C═O).

### N-octyl-7-Oxabicyclo(2.2.1)heptane-2,3-dicarboximide (6)

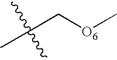

Octyl amine (0.384 g, 2.97 mmol) was added dropwise to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was recrystallised to afford the pure N-octyl-7-Oxabicyclo(2.2.1)heptane-2,3-dicarboximide in a 72% yield. Mp: 36-37° C.; ¹H NMR: (CDCl₃): 0.85 (t, J=6.9 Hz, 3H, CH₃), 1.24 (s, 10H, 5×CH₂), 1.52-1.59 (m, 4H, 2×CH₂), 1.81-1.84 (m, 2H, CH₂), 2.82 (s, 2H, 2×CH), 3.43 (t, J=7.5 Hz, 2H, CH₂), 4.84 (q, J=1.0 Hz, 2H, 2×CH); ¹³C NMR: (CDCl₃): 13.4 (CH₃), 22.0 (CH₂), 21.9 (CH₂), 26.1 (CH₂), 27.0 (CH₂), 28.0 (2×CH₂), 28.4 (CH₂), 28.5 (CH₂), 31.1 (CH₂), 38.5 (CH₂), 49.3 (2×CH) 78.4 (CH₂), 176.5 (2×C═O).

### N-decyl-7-Oxabicyclo(2.2.1)heptane-2,3-dicarboximide (7)

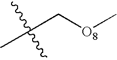

Decyl amine (0.467 g, 2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-decyl-7-Oxabicyclo(2.2.1)heptane-2,3-dicarboximide in a 68% yield. Mp: 30-31° C.; ¹H NMR: (CDCl₃): 0.88 (t, J=6.7 Hz, 3H, CH₃), 1.26 (s, 14H, 7×CH₂), 1.52-1.59 (m, 4H, 2×CH₂), 1.82-1.85 (m, 2H, CH₂), 2.81 (s, 2H, 2×CH), 3.44 (t, J=7.3 Hz, 2H, CH₂), 4.84 (t, J=2.1 Hz, 2H, 2×CH); ¹³C NMR: (CDCl₃): 14.0 (CH₃), 22.7 (CH₂), 26.7 (CH₂), 27.6 (CH₂), 28.7 (2×CH₂), 29.1 (CH₂), 29.3 (CH₂), 29.5 (CH₂), 32.0 (CH₂), 39.2 (CH₂), 50.5 (2×CH), 79.1 (2×CH), 177.2 (2×C═O).

### N-(3-butenyl)-7-Oxabicyclo(2.2.1)heptane-2,3-dicarboximide (8)

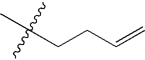

2-Buten-1-amine (2.97 mmol) was added to a stirred solution of 7-oxabicyclo(2.2.1)Heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-(3-butenyl)-7-Oxabicyclo(2.2.1)heptane-2,3-dicarboximide in a 31% yield. Mp: 64-65° C.; ¹H NMR (DMSO-d6): 1.61 (brs, 4H, 2×CH₂), 2.17 (q, J=6.9 Hz, 21-1, CH₂), 3.00 (s, 2H, 2×CH), 3.37 (t, J=6.9 Hz, 2H, CH₂), 4.66 (s, 2H, 1×CH), 4.99 (m, 2H, CH═CH₂), 5.65 (m, 1H, CH═CH₂); ¹³C NMR (DMSO-d6): 28.8 (2×CH₂), 32.2 (CH₂), 38.2 (CH₂), 50.2 (2×CH), 79.2 (CH), 79.2 (CH), 117.9 (CH═CH₂), 135.5 (CH═CH₂), 170.1 (2×C═O).

### N-Butanoic acid-7-Oxabicyclo(2.2.1)heptane-2,3-dicarboximide (13)

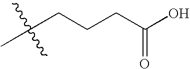

Aminobutanoic acid (2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-Butanoic acid-7-Oxabicyclo(2.2.1)heptane-2,3-dicarboximide in a 45% yield. Mp: 162-164° C.; ¹H NMR (CDCl₃): 4.86 (2H, q J=2.3 Hz), 3.54 (2H, t J=6.8 Hz), 2.86 (2H, s), 2.33 (2H, t J=7.4 Hz), 1.91-1.83 (4H, m), 1.58 (2H, m); ¹³C NMR (CDCl₃): 177.1, 176.7, 78.5, 49.3, 37.5, 30.4, 28.0, 22.0.

### N-Hexanoic acid-7-Oxabicyclo(2.2.1)heptane-2,3-dicarboximide (14)

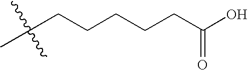

Aminohexanoic acid (2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-Hexanoic acid-7-Oxabicyclo(2.2.1)heptane-2,3-dicarboximide in a 56% yield. M.W.=281.30; M.p: 110-112° C.; ¹H NMR (CDCl₃): 4.86 (2H, q J=2.2 Hz), 3.45 (2H, t J=7.23), 2.86 (2H, s), 2.32 (2H, t J=7.44), 1.83 (2H, m), 1.60 (6H, m), 1.30 (2H, m); ¹³C NMR (CDCl₃): 178.6, 176.8, 78.5, 49.3, 38.2, 33.2, 28.0, 26.6, 25.4, 23.5.

### N-Ethyl alcohol-7-Oxabicyclo(2.2.1)heptane-2,3-dicarboximide (15)

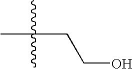

2-Aminoethanol (2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-Ethyl alcohol-7-Oxabicyclo(2.2.1)heptane-2,3-dicarboximide in a 80% yield. Mp: 164-166° C.; ¹H NMR (CDCl₃): 4.86 (2H, q J=2.1 Hz), 3.70 (2H, t J=4.8 Hz), 3.64 (2H, q J=3.6 Hz), 2.89 (2H, s), 3.01 (1H, br OH), 1.84 (2H, m), 1.61 (2H, m); ¹³C NMR (CDCl₃): 177.1, 78.6, 59.5, 49.4, 41.3, 27.9.

### N-2-Methoxypropyl-1-ol-7-Oxabicyclo(2.2.1)heptane-2,3-dicarboximide (16)

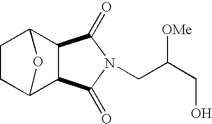

Amine (2.97 mmol) was added to a stirred solution of 7-oxabicyclo(2.2.1)heptane-2,3-dicarboxylic anhydride (1.0 g, 2.97 mmol) and triethylamine (1.5 mL) in toluene (15 mL). The resulting solution was refluxed for 36 h, cooled and diluted with EtOAc (45 mL). Following a standard workup, the resulting crude was purified to afford the pure N-2-Methoxypropyl-1-ol-7-Oxabicyclo(2.2.1)heptane-2,3-dicarboximide in a 91% yield. Mp: 96-97° C.; ¹H NMR (DMSO-d6): 1.60-1.63 (m, 2H, CH₂), 1.85-1.88 (m, 21-1, CH₂), 2.91 (s, 2H, 2×CH), 3.33-3.39 (m, 2H, CH₂), 3.38 (s, 3H, OCH₃), 3.60-3.68 (m, 2H, CH₂), 3.94-3.96 (m, 1H, CH), 4.88-4.89 (m, 2H, 2×CH); ¹³C NMR (DMSO-d6): 28.0 (2×CH₂), 41.7 (CH), 49.4 (2×CH), 58.7 (CH), 67.7 (CH₂), 73.5 (CH₂), 78.6 (2×CH), 177.0 (2×C═O).

### 4-Ethyl-7-Oxabicyclo(2.2.1)heptane-2,3-dicarboximide (9e)

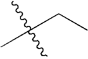

Isolated as a white solid, 81% yield, mp 166-169° C. ¹H NMR (DMSO-d₆): 1.09 (3H, t, J=7.21 Hz), 1.55 (2H, m), 1.80 (2H, m), 2.80 (2H, s), 3.46 (2H, q, J=7.1 Hz), 4.81 (2H, dd, J=2.4, 3.1 Hz). ¹³C NMR (DMSO-d₆): 12.9, 28.5, 33.9, 49.9, 79.0, 176.9.

### 4-Sec-butyl-7-Oxabicyclo(2.2.1)heptane-2,3-dicarboximide (12a)

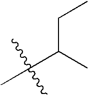

Isolated as a white solid, 98%, mp: 44-46° C. ¹H NMR (DMSO-d₆): 0.79 (3H, t, J=7.5 Hz), 1.30 (3H, d, J=7.0 Hz), 1.64-1.52 (2H, m), 1.80-1.92 (4H, m), 2.77 (2H, m), 4.03 (1H, m), 4.82 (2H, s). ¹³C NMR (DMSO-d₆): 10.9, 17.5, 25.9, 28.6, 49.5, 50.1, 79.2, 177.5.

### 4-Hexyl-7-Oxabicyclo(2.2.1)heptane-2,3-dicarboximide (13a)

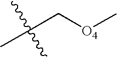

Isolated as a colourless oil 87% yield. ¹H NMR (CDCl₃): δ 0.85 (4H, m), 1.25 (8H, m), 1.57 (4H, m), 1.84 (2H, m), 2.84 (2H, s), 3.43 (2H, t, J=7.1 Hz), 4.85 (2H, q J=2.4 Hz). ¹³C NMR (CDCl₃): δ 13.4, 21.9, 25.7, 26.9, 28.0, 30.7, 38.5, 49.3, 78.5, 176.7.

### 4-Cyclohexyl-7-Oxabicyclo(2.2.1)heptane-2,3-dicarboximide (14a)

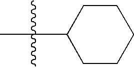

Isolated as a white solid, 82%, mp 97-99° C. ¹H NMR (DMSO-d₆): 1.16 (4H, m), 1.53 (6H, m), 1.77 (4H, m), 2.05 (2H, m), 2.73 (2H, s), 3.86 (1H, m), 4.78 (2H, m). ¹³C NMR (DMSO-d₆): 25.0, 25.8, 28.5, 28.6, 49.4, 51.9, 79.1, 177.3.

### 4-Dodecyl-7-Oxabicyclo(2.2.1)heptane-2,3-dicarboximide (17a)

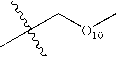

Isolated as a white solid, 82% yield, mp 35-37° C. ¹H NMR (CDCl₃): δ 0.87 (4H, m), 1.24 (20H, s), 1.85-1.82 (2H, m), 1.60-1.50 (4H, m), 2.83 (2H, s), 3.44 (2H, t J=7.3 Hz), 4.85 (2H, q J=2.3 Hz). ¹³C NMR (CDCl₃): δ 13.5, 22.1, 26.1, 27.0, 28.0, 28.5, 28.8, 28.9, 29.0, 38.5, 49.3, 78.5, 176.7.

### 4-Tetradecyl-7-Oxabicyclo(2.2.1)heptane-2,3-dicarboximide (18a)

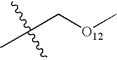

Isolated as a white solid, 79% yield, mp 45-46° C. ¹H NMR (CDCl₃): δ 0.86 (2H, m), 1.23 (20H, s), 1.56 (2H, m), 1.83 (2H, quin, J=4.5 Hz), 2.82 (2H, s), 3.42 (2H, t, J=7.3 Hz), 4.84 (2H, q, J=2.2 Hz). ¹³C NMR (CDCl₃): δ 13.5, 22.1, 26.1, 28.0, 28.5, 29.0, 29.1, 38.5, 49.3, 78.5, 176.7.

### 4-Octadecyl-7-Oxabicyclo(2.2.1)heptane-2,3-dicarboximide (19a)

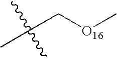

Isolated as a white solid, 80% yield, mp 62-64° C. ¹H NMR (CDCl₃): δ 1.58 (2H, m), 1.83-1.91 (4H, m), 2.86 (2H, s), 2.33 (2H, t, J=7.4 Hz), 3.54 (2H, t, J=6.8 Hz), 4.86 (2H, q, J=2.3 Hz). ¹³C NMR (CDCl₃): δ 22.0, 28.0, 30.4, 37.5, 49.3, 78.5, 176.7, 177.1.

### 4-Allyl-7-Oxabicyclo(2.2.1)heptane-2,3-dicarboximide (20a)

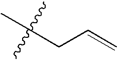

Isolated as a white solid, 75% yield, mp 116-117° C. ¹H NMR (CDCl₃): δ 1.55-1.62 (2H, m), 1.85-1.88 (2H, m), 2.89 (2H, s), 4.08 (2H, dd, J=4.0, 1.3 Hz), 4.88-4.90 (2H, m), 5.16-5.22 (2H, m), 5.71-5.76 (1H, m). ¹³C NMR (DMSO-d₆): δ 28.0 (2C), 40.3, 49.4, 78.4, 116.9, 129.8, 176.0 (2C).

### 4-Oxiranylmethyl-7-Oxabicyclo(2.2.1)heptane-2,3-dicarboximide (22a)

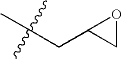

m-Chloroperbenzoic acid (2.15 g, 77% in water, 9.66 mmol) was added in one portion to cooled and magnetically stirred solution 0° C. of allyl-N-norcanthaimide (1.0 g, 4.83 mmol) in CH₂Cl₂ (20 mL). The resulting solution was warmed to room temperature, stirred for 16 h before being diluted with CH₂Cl₂ (30 mL) and washed with NaHCO₃ (3×10 mL, sat solution). The organic layer was dried (Na₂SO₄), filtered and concentrated under reduced pressure to afford a white solid. Flash chromatography (50% EtOAc/Hexane) afforded the norantharimide epoxide (820 mg) as a white solid. 76% yield, mp 83-84° C. ¹H NMR (CDCl₃): δ 1.53-1.59 (2H, m), 1.79-1.82 (2H, m), 2.51-2.54 (1H, m), 2.68 (1H, t, J=4.1 Hz), 2.86 (2H, s), 3.04-3.06 (1H, m), 3.55 (1H, dd, J=14, 4.6 Hz), 3.64 (1H, dd, J=14, 4.6 Hz), 4.80-4.82 (2H, m). ¹³C NMR (CDCl₃): δ 27.9 (2C), 39.8 (2C), 45.2, 47.8, 49.3, 49.4 (2C) 78.4, 176.3 (2C).

### 4-(3-Hydroxypropyl)-7-Oxabicyclo(2.2.1)heptane-2,3-dicarboximide (25a)

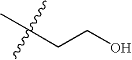

Isolated as a white solid, 80% yield, mp 164-166° C. ¹H NMR (CDCl₃): δ 1.61 (2H, m), 1.84 (2H, m), 2.89 (2H, s), 3.01 (1H, brs), 3.64 (2H, q, J=3.6 Hz), 3.70 (2H, t, J=4.8 Hz), 4.86 (2H, q, J=2.1 Hz). ¹³C NMR (CDCl₃): δ 27.9, 41.3, 49.4, 59.5, 78.6, 177.1.

### 4-(6-Hydroxyhexyl)-7-Oxabicyclo(2.2.1)heptane-2,3-dicarboximide (26a)

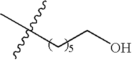

Isolated as a white solid, 62% yield, mp 56-57° C. ¹H NMR (CDCl₃): δ 1.27 (4H, m), L48 (2H, m), 1.57 (2H, m), 2.15 (2H, m), 2.82 (2H, s), 3.41 (2H, m), 3.54 (2H, m), 4.80 (2H, m). ¹³C NMR (CDCl₃): δ 24.5, 25.6, 26.8, 27.9, 31.8, 38.3, 49.3, 61.9, 78.5, 176.8.

### 4-(2-Hydroxy-1-methylethyl)-7-Oxabicyclo(2.2.1)heptane-2,3-dicarboximide (27a)

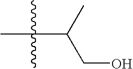

Isolated as a yellow solid, yield 22%, mp 104-107° C. ¹H NMR (DMSO-d₆): 1.28 (3H, d, J=7.1 Hz), 1.58 (2H, m), 1.84 (m), 2.14 (1H, s), 2.83 (2H, s), 3.70 (1H, m), 3.80 (1H, m), 4.26 (1H, m), 4.84 (2H, m). ¹³C NMR (DMSO-d₆): 13.8, 28.5, 28.6, 49.6, 49.7, 50.4, 63.5, 79.3, 177.9.

### Synthesis of 4-(2-Hydroxy-1,1-dimethylethyl)-7-Oxabicyclo(2.2.1)heptane-2,3-dicarboximide (28a)

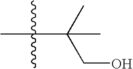

Isolated as a pale yellow solid, yield 46%, mp 134-136° C. ¹H NMR (DMSO-d₆): δ 1.39 (6H, s), 1.52 (2H, m), 1.78 (2H, m), 2.72 (2H, s), 3.52 (1H, bs), 3.71 (2H, s), 4.78 (2H, dd, J=2.3, 3.1 Hz). ¹³C NMR (DMSO-d₆): δ 22.1, 28.4, 49.6, 63.0, 68.9, 79.5, 179.1.

### Synthesis of 4-(1-Hydroxymethylpropyl)-7-Oxabicyclo(2.2.1)heptane-2,3-dicarboximide (29a)

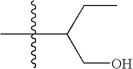

Isolated as a pale yellow oil, yield 49%. ¹H NMR (DMSO-d₆): δ 0.79 (3H, t, J=7.4 Hz), 1.38-1.79 (6H, m), 2.81 (2H, q, J=11.6, 7.0 Hz), 3.01 (1H, bs), 3.63 (1H, m), 3.87 (1H, m), 4.05 (1H, m), 4.79 (2H, s). ¹³C NMR (DMSO-d₆): δ 10.3, 20.7, 28.4, 28.5, 49.4, 49.6, 56.5, 62.1, 79.2, 178.2.

### 4-(3,5-Dioxo-10-oxa-4-azatricyclo[5.2.4]dec-4-yl)-butyric acid (30a)

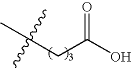

Isolated as a white solid, 45% yield, mp 162-164° C. ¹H NMR (CDCl₃): δ 1.58 (2H, m), 1.83-1.91 (4H, m), 2.33 (2H, t, J=7.4 Hz), 2.86 (2H, s), 3.54 (2H, t, J=6.8 Hz), 4.86 (2H, q, J=2.3 Hz). ¹³C NMR (CDCl₃): δ 22.0, 28.0, 30.4, 37.5, 49.3, 78.5, 176.7, 177.1.

### 6-(3,5-Dioxo-10-oxa-4-azatricyclo[5.2.1]dec-4-yl)-hexanoic acid (31a)

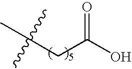

Isolated as a white solid, 56% yield, mp 110-112° C. ¹H NMR (CDCl₃): δ 1.30 (2H, m), 1.60 (6H, m), 1.83 (2H, m), 2.32 (2H, t, J=7.44), 2.86 (2H, s), 3.45 (2H, t, J=7.23), 4.86 (2H, q, J=2.2 Hz). ¹³C NMR (CDCl₃): δ 23.5, 25.4, 26.6, 28.0, 33.2, 38.2, 49.3, 78.5, 176.8, 178.6.

### 4-(2,3-Dihydroxypropyl)-10-oxa-4-azatricyclo[5.2.1]decane-3,5-dione (23a)

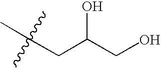

OsO₄ (120 mg of a 2.5% solution in T-BuOH, 0.012 mmol, 0.5 mol %) was added dropwise to a magnetically stirred solution of allyl substituted (500 mg, 2.41 mmol), N-methylmorpholine-N-oxide (310 mg, 2.65 mmol) in acetone/water (5 mL/2 mL). The resulting solution was heated at 80° C. for 16 h before being diluted with ether (100 mL) and washed with water (3×20 mL). The organic layer was concentrated under reduced pressure to afford a brown solid. Flash chromatography (70% EtOAc/hexanes) afforded a pale white solid which was recrystallised from EtOAc to afford a white crystalline solid in a 34% yield. ¹H NMR (DMSO-d₆): δ 1.62 (4H, s), 3.00 (2H, s), 3.23-3.32 (2H, m), 3.63 (1H, q, J=5.7 Hz), 4.51 (1H, t, J=5.7 Hz), 4.66 (2H, s), 4.73 (1H, d, J=4.6 Hz). ¹³C NMR (DMSO-d₆): 27.8 (2C), 41.8, 49.3 (2C) 63.9, 67.9, 78.3 (2C), 177.4.

### 4-(3-Hydroxy-2-methoxypropyl)-10-oxa-4-azatricyclo[5.2.1]decane-3,5-dione (24a)

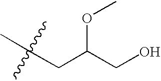

(1S)-(+)-10-Camphorsulfonic acid (15 mg, 0.06 mmol) was added to a magnetically stirred solution of epoxide (220 mg, 0.98 mmol) in methanol (4 mL). The resulting solution was warmed to 35° C. and stirred for 16 h before being concentrated under reduced pressure. The resulting clear oil was subjected to flash chromatography (70% EtOAc/Hexanes) to afford a white solid, 78%, mp 95-96° C. ¹H NMR (DMSO-d₆): δ 1.60-1.63 (2H, m), 1.85-1.88 (2H, m), 2.91 (2H, s), 3.33-3.39 (2H, m), 3.38 (3H, s), 3.60-3.68 (2H, m), 3.94-3.96 (1H, m), 4.88-4.89 (2H, m). ¹³C NMR (DMSO-d₆): δ 28.0 (2C), 41.7, 49.4 (2C), 58.7, 67.7, 73.5, 78.6 (2C), 177.0 (2C).

### 4-Morpholin-4-yl-7-Oxabicyclo(2.2.1)heptane-2,3-dicarboximide (32a)

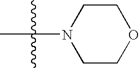

Isolated as a yellow solid, yield 43%, mp 169-171° C. ¹H NMR (DMSO-d₆): δ 1.53 (2H, m), 1.78 (2H, m), 2.73 (2H, s), 3.18 (4H, t, J=4.5 Hz), 3.71 (4H, t, J=4.7 Hz), 4.78 (2H, m); ¹³C NMR (DMSO-d₆): δ 28.5, 47.9, 51.2, 66.7, 79.1, 175.1.

### 4-(2-Morpholin-4-ylethyl)-7-Oxabicyclo(2.2.1)heptane-2,3-dicarboximide dione (33a)

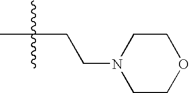

Isolated as an orange brown solid, yield 37%, mp 109-111° C.; ¹H NMR (DMSO-d₆): 1.56 (2H, m), 1.80 (2H, m), 2.43-2.48 (6H, m), 2.84 (2H, s), 3.55 (2H, t, J=4.5 Hz), 3.60 (4H, t, J=4.5 Hz), 4.82 (2H, m). ¹³C NMR (DMSO-d₆): 28.6, 36.6, 49.9, 53.4, 55.1, 67.0, 79.0, 177.1.

### 4-(3-Morpholin-4-ylpropyl)-7-Oxabicyclo(2.2.1)heptane-2,3-dicarboximide (34a)

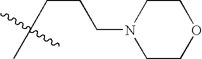

Isolated as an orange/brown oil, 7%. ¹H NMR (DMSO-d₆): δ 1.55 (2H, m), 1.70 (2H, s), 1.81 (2H, m), 2.81 (2H, s), 2.30 (2H, t), 2.37 (4H, m), 3.48 (2H, t, J=4.5 Hz), 3.65 (4H, t, J=4.7 Hz), 4.82 (2H, m). ¹³C NMR (DMSO-d₆): δ 24.2, 28.5, 37.2, 49.9, 51.3, 53.4, 55.8, 66.8, 79.0, 177.2.

### 4-Phenyl-7-Oxabicyclo(2.2.1)heptane-2,3-dicarboximide (35a)

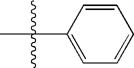

Isolated as a bone coloured solid, 79% yield, mp 171-172° C. ¹H NMR: (CDCl₃): δ 1.63-1.65 (2H, m), 1.87-1.90 (2H, m), 3.01 (2H, s), 4.97 (2H, q, J=1.1 Hz), 7.25-7.28 (2H, m), 7.42-7.45 (2H, m). ¹³C NMR: (CDCl₃): 28.1 (2C), 49.5 (2C), 78.9, 125.0 (2C), 128.1, 128.5, 175.7.

### 4-(4-Hydroxyphenyl)-10-oxa-4-azatricyclo[5.2.1]decane-3,5-dione (36a)

Isolated as a white solid, 45% yield, 172-173° C. ¹H NMR (CDCl₃): δ 1.56-1.62 (4H, m), 3.01 (2H, s), 4.98 (2H, q, J=0.9 Hz), 6.86 (2H, d, J=8.1 Hz), 7.11 (21-1, d, J=8.1 Hz). ¹³C NMR (DMSO-d₆): δ 28.1 (2C), 49.4, 78.9, 115.4 (2C), 127.4 (2C), 181.5 (2C).

### 4-(4-Nitrophenyl)-7-Oxabicyclo(2.2.1)heptane-2,3-dicarboximide (37a)

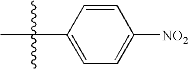

Isolated as an orange yellow solid, 76% yield, mp 207-209° C. ¹H NMR (CDCl₃): δ 1.66-1.69 (2H, m), 1.90-1.94 (2H, m), 3.07 (2H, s), 4.99 (21-1, q, J=0.9 Hz), 7.55 (2H, d, J=7.1 Hz), 8.29 (2H, d, J=7.1 Hz). ¹³C NMR (CDCl₃): δ 28.0 (2C), 49.5 (2C), 79.1, 123.7, 126.4, 136.8, 146.5, 174.9.

### 4-(3,5-Dioxo-10-oxa-4-azatricyclo[5.2.1]dec-4-yl)benzoic acid (38a)

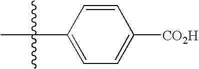

Isolated as a white solid, 36% yield, mp 275-277° C. ¹H NMR (CDCl₃): 1.63 (2H, m), 1.88 (2H, m), 3.00 (2H, s), 4.96 (2H, q, J=1.9 Hz), 7.30 (2H, d, J=8.1 Hz), 8.13 (2H, d, J=8.4 Hz). ¹³C NMR (CDCl₃): 28.0, 49.5, 78.9, 125.3, 129.8, 132.2, 134.3, 160.1, 175.3.

### 4-Benzyl-10-oxa-4-azatricyclo[5.2.1]decane-3,5-dione (39a)

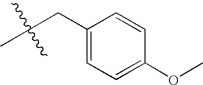

Isolated as a white solid, yield 91%, mp 97-100° C. ¹H NMR (DMSO-d₆): δ 1.21 (2H, m), 1.54 (2H, m), 1.77 (2H, m), 2.81 (2H, s), 4.56 (2H, s), 4.81 (2H, m), 7.22 (5H, m). ¹³C NMR (DMSO-d₆): δ 28.4, 42.3, 49.8, 78.9, 127.5, 127.8, 128.4, 135.3, 176.7.

### 4-(4-Methoxybenzyl)-7-Oxabicyclo(2.2.1)heptane-2,3-dicarboximide (40a)

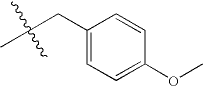

Isolated as a white solid, mp 83-85° C. ¹H NMR (CDCl₃): δ 1.60 (2H, m), 1.85 (2H, m), 2.84 2H, s), 3.77 (3H, s), 4.56 (2H, s), 4.87 (2H, q J=2.1 Hz), 6.81 (2H, d J=6.7 Hz), 7.25 (2H, d, J=6.6 Hz). ¹³C NMR (CDCl₃): δ 28.0, 41.4, 49.5, 54.6, 78.5, 113.4, 127.3, 129.1, 158.7, 176.2.

### 4-(3,4-Dimethoxybenzyl)-7-Oxabicyclo(2.2.1)heptane-2,3-dicarboximide (41a)

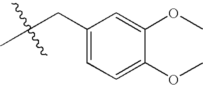

Isolated as a white solid, 76% yield, mp 155-157° C. ¹H NMR (CDCl₃): δ 1.60 (2H, m), 1.84 (2H, m), 2.86 (2H, s), 3.83 (3H, s), 3.84 (3H, s), 4.56 (2H, s), 4.87 (2H, q J=2.4 Hz), 6.77 (1H, m), 6.89 (2H, m). ¹³C NMR (CDCl₃): δ 28.0, 41.7, 49.5, 55.3, 78.5, 110.7, 110.9, 120.1, 127.6, 148.1, 148.5, 176.3.

### 3-[3-(Benzyloxy)pyridin-2-yl]-10-oxa-4-azatricyclo[5.2.1]decane-3,5-dione (77)

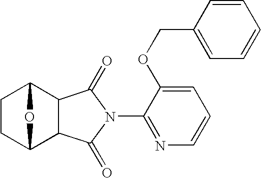

Purification afforded (77) (0.21 g, 20%) as a white crystalline solid. Mp 99-102° C.; ¹H NMR (300 MHz, DMSO-d₆) δ 7.48 (m, 2H), 7.34 (m, 4H), 7.06 (dd, J=7.8, 1.2 Hz, 1H), 6.46 (dd, J=7.7, 5.0 Hz, 1H), 5.10 (s, 2H), 4.65 (t, J=2.4 Hz, 2H), 2.89 (s, 2H), 1.52-1.49 (m, 4H); ¹³C NMR (75 MHz, DMSO-d₆) δ 172.4, 150.8, 140.4, 138.3, 136.8, 128.3, 127.7, 127.4, 116.7, 111.9, 77.6, 68.9, 51.5, 28.5; Calculated for C₂₀H₁₈N₂O₄; Exact Mass: 350.1267; m/e: 350.1267 (100.0%), 351.1300 (22.2%), 352.1334 (2.4%); Elemental C, 68.56; H, 5.18; N, 8.00; O, 18.27.

### 4-{4-[2-(Dimethylamino)ethoxy]phenyl}-10-oxa-4-azatricyclo[5.2.1]decane-3,5-dione (78)

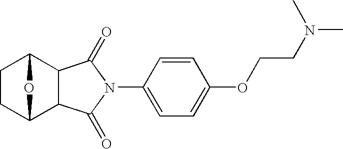

Purification afforded (78) (1.67 g, 76%) as a white crystalline solid. Mp 145-146° C.; ¹H NMR (300 MHz, DMSO-d₆) δ 6.64 (d, J=8.8 Hz, 2H), 6.48 (d, J=8.8 Hz, 2H), 4.65 (t, J=2.4 Hz, 2H), 3.91 (t, 5.8 Hz, 2H), 2.85 (s, 2H), 2.68 (t, J=5.8 Hz, 2H), 2.27 (s, 6H), 1.52-1.49 (m, 4H). ¹³C NMR (75 MHz, DMSO-d₆) δ 172.7, 149.5, 142.5, 115.3, 114.8, 78.2, 65.6, 57.4, 52.0, 45.0, 28.6; Calculated for C₁₈H₂₂N₂O₄; Exact Mass: 330.1580; m/e: 330.1580 (100.0%), 331.1613 (20.0%), 332.1647 (1.9%); Elemental C, 65.44; H, 6.71; N, 8.48; O, 19.37

### 4,{2,[2-(Dimethylamino)ethoxy]phenyl}-10-oxa-4-azatricyclo[5.2.1]decane-3,5-dione (79)

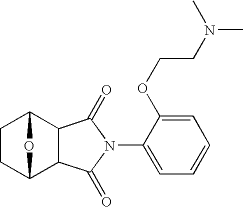

Purification afforded (79) (0.65 g, 48%) as a white crystalline solid. Mp 113-115° C.; ¹H NMR (300 MHz) (DMSO-d₆) δ 7.35 (m, 2H), 7.12 (d, J=8.0 Hz, 1H), 6.98 (t, J=8.1 Hz, 1H), 4.67 (t, J=2.4 Hz, 2H), 4.20 (t, J=5.2 Hz, 2H), 3.96 (s, 2H), 2.71 (t, J=5.2 Hz, 2H), 2.24 (s, 6H), 1.53-1.45 (m, 4H). ¹³C NMR (75 MHz, DMSO-d₆) 8 ¹³C NMR (75 MHz, DMSO-d₆) δ 173.7, 156.5, 130.5, 130.2, 123.1, 120.8, 113.0, 79.2, 65.1, 56.9, 53.1, 44.1, 28.9; Calculated for C₁₈H₂₂N₂O₄; Exact Mass: 330.1580; m/e: 330.1580 (100.0%), 331.1613 (20.0%), 332.1647 (1.9%); Elemental C, 65.44; H, 6.71; N, 8.48; O, 19.37.

### 4-(2-Aminobenzyl)-7-Oxabicyclo(2.2.1)heptane-2,3-dicarboximide (42a)

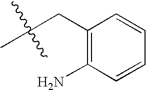

Isolated as a yellow solid, 43% yield, mp 159-160° C. ¹H NMR (DMSO-d₆): δ 1.54-1.46 (4H, m), 2.74 (2H, s), 3.84 (2H, s), 4.71 (2H, s), 6.54 (1H, t), 6.68 (1H, d), 7.09-7.00 (2H, m). ¹³C NMR (DMSO-d₆): δ 29.0, 53.7, 79.8, 115.3, 116.1, 118.7, 128.9, 129.8, 146.8, 174.1.

### Bis-3,6-epoxycyclohexane-1,2-dicarboximido)-trimethylene (Bis-norcantharimide-propyl linker) (43a)

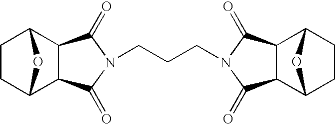

Isolated as a white solid, 80% yield. ¹H NMR (DMSO-d₆): δ 1.58 (2H, quin), 1.85 (4H, m), 2.86 (2H, s), 3.43 (2H, m), 4.86 (2H, m). ¹³C NMR (DMSO-d₆): δ 24.7, 28.0, 35.6, 49.4, 78.5, 176.5.

### Bis-3,6-epoxycyclohexane-1,2-dicarboximido)-dodecylmethyle (Bis-norcantharimide-dodecyl linker) (44a)

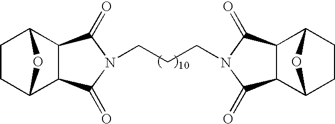

Isolated as a white solid, 62% yield ¹H NMR (CDCl₃): δ 1.22 (10H, s), 1.59-1.51 (4H, m), 1.85 (2H, m), 2.84 (2H, s), 3.43 (2H, t, J=7.3 Hz), 4.85 (2H, q, J=2.2 Hz). ¹³C NMR (CDCl₃): δ 26.0, 27.0, 28.5, 28.8, 28.9, 38.5, 49.3, 78.5, 176.6.

### Example 3

### Preparation of 7-Oxabicyclo(2.2.1)heptane-2-carboxylic Acid Derivatives

### General Synthetic Scheme for Carboxylic Acid Derivatives

The 7-Oxabicyclo(2.2.1)heptane-2-carboxylic acid derivatives were prepared by treating the appropriate anhydrides directly with alcohols or amines. In a typical procedure the parent anhydride (1 mmol) was dissolved in THF (20 mL) and to this the requisite amine (1.1 equiv) added portionwise (for solids) or dropwise (in THF (5 mL) for liquids. After complete reaction (TLC) the solid was filtered, or the solvent evaporated (in those cases when no precipitate was evident) and the product purified by flash chromatography (hexanes:ethyl acetate).

### 3-(Butylcarbamoyl)-7-Oxabicyclo(2.2.1)heptane-2-carboxylic acid (9)

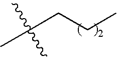

¹H NMR (DMSO-d₆): 0.84 (t, J=7.2 Hz, 3H), 1.26-1.34 (m, 4H), 1.46-1.52 (m, 4H), 2.81 (s, 2H), 2.96 (q, J=5.9 Hz, 2H), 4.45 (d, J=4.0 Hz, 1H), 4.71 (d, J=3.6 Hz, 1H), 7.24 (brs, 1H); ¹³C NMR (DMSO-d₆): 13.5, 19.4, 28.3, 28.7, 31.0, 51.5, 53.1, 76.7, 78.7, 170.2, 172.2; Mp: 101-104° C.

### 3-Propylcarbamoyl-7-oxabicyclo[2.2.1]heptane-2-carboxylic acid (9a)

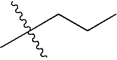

¹H NMR (DMSO-d₆): 0.80 (t, J=7.5 Hz, 3H), 1.34-1.36 (m, 2H), 1.41-1.51 (m, 4H), 2.81 (s, 2H), 2.90 (q, J=5.8 Hz), 4.45 (d, J=3.1 Hz, 1H), 4.71 (d, J=2.0 Hz, 1H), 7.23 (d, 1H); ¹³C NMR (DMSO-d₆): 11.0, 22.2, 28.3, 28.7, 40.3, 51.4, 53.1, 76.7, 78.7, 170.2, 172.2; Mp: 125° C.

### 3-Hexylcarbamoyl-7-oxabicyclo[2.2.1]heptane-2-carboxylic acid (9b)

¹H NMR (CDCl₃): 0.87 (3H, t), 1.27 (8H, m), 1.30 (2H, m), 1.57 (2H, m), 1.80 (2H, m), 3.03 (2H, q), 3.17 (2H, m), 4.67 (1H, d J=4.8 Hz), 5.03 (1H, d J=4.6 Hz), 6.64 (1H, t J=5.5. Hz); ¹³C NMR (CDCl₃): 13.4, 21.9, 25.7, 28.4, 26.9, 28.8, 30.7, 38.5, 51.4, 53.0, 76.7, 78.7, 170.3, 172.3; Mp: 126-128° C.

### 3-Octylcarbamoyl-7-oxabicyclo[2.2.1]heptane-2-carboxylic acid (9c)

¹H NMR (DMSO-d₆): 1.22-1.24 (m, 11H), 1.49-1.60 (m, 6H), 1.82-1.85 (m, 2H), 2.83 (s, 2H), 3.43 (t, J=7.5 Hz), 4.84 (q, J=2.1 Hz, 2H). ¹³C NMR (DMSO-d₆): 26.0 (CH₃), 27.0 (CH₂), 28.0 (2×CH₂), 28.5 (CH₂), 28.8 (CH₂), 28.9 (CH₂), 38.5 (CH₂), 49.3 (CH), 49.3 (CH), 78.5 (CH₂), 176.6 (2×C═O); Mp: 131° C.

### 3-(Decylcarbamoyl)-7-Oxabicyclo(2.2.1)heptane-2-carboxylic acid (10)

Mp: 107-108° C.; ¹H NMR (DMSO-d6): 0.84 (t, J=6.9 Hz, 3H, CH₃), 1.22 (s, 14H, 7×CH₂), 1.31-1.33 (m, 2H, CH₂), 1.46-1.51 (m, 4H, 2×CH₂), 2.81 (s, 2H, 2×CH), 2.94 (q, J=5.3 Hz, CH₂), 4.43 (d, J=3.7 Hz, 1H, CH), 4.69 (d, J=3.7 Hz, 1H, CH), 7.26 (brs, 1H, NH); ¹³C NMR (DMSO-d6): 13.8 (CH₃), 21.9 (CH₂), 26.3 (CH₂), 28.3 (CH₂), 28.6 (CH₂), 28.7 (CH₂), 28.8 (CH₂), 28.9 (CH₂), 31.1 (CH₂), 51.4 (CH), 53.0 (CH), 76.7 (CH), 78.7 (CH), 170.2 (CON), 172.2 (COOH).

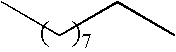

¹H NMR (DMSO-d6): 0.84 (t, J=6.9 Hz, 3H, CH3), 1.22 (s, 14H, 7×CH₂), 1.31-1.33 (m, 2H, CH₂), 1.46-1.51 (m, 4H, 2×CH2), 2.81 (s, 2H, 2×CH), 2.94 (q, J=5.3 Hz, CH₂), 4.43 (d, J=3.7 Hz, 1H, CH), 4.69 (d, J=3.7 Hz, 1H, CH), 7.26 (brs, 1H, NH). ¹³C NMR (DMSO-d6): 13.8 (CH3), 21.9 (CH2), 26.3 (CH2), 28.3 (CH₂), 28.6 (CH2), 28.7 (CH2), 28.8 (CH2), 28.9 (CH2), 31.1 (CH2), 51.4 (CH), 53.0 (CH), 76.7 (CH), 78.7 (CH), 170.2 (CON), 172.2 (COOH).

### 3-Dodecylcarbamoyl-7-oxabicyclo[2.2.1]heptane-2-carboxylic acid (10a)

¹H NMR (CDCl₃): 6.67 (1H, t, J=5.3 Hz), 5.03 (1H, d, J=3.5 Hz), 4.66 (1H, d, J=4.3 Hz), 3.18-3.00 (4H, m), 1.80 (2H, m), 1.57 (2H, m), 1.45 (2H, m), 1.25 (20H, s), 0.87 (3H, t); ¹³C NMR 172.3, 170.3, 78.7, 76.7, 53.0, 51.4, 38.5, 30.7, 29.0, 28.9, 28.8, 28.5 28.6, 28.4, 26.9, 25.7, 21.9, 13.4; Mp: 115-117° C.

### 3-Tetradecylcarbamoyl-7-oxabicyclo[2.2.1]heptane-2-carboxylic acid (10b)

¹H NMR (CDCl₃): 0.87 (3H, t), 1.24 (2H, s), 1.44 (2H, m), 1.54 (2H, m), 1.78 (2H, m), 3.01-3.16 (4H, m), 4.65 (1H, d), 5.01 (1H, d), 6.76 (1H, t); ¹³C NMR (CDCl₃): 13.0, 21.9, 25.7, 26.9, 28.4, 28.5, 28.6, 28.8, 28.9, 29.0, 30.7, 38.5, 51.4, 53.0, 76.7, 78.7, 170.3, 172.3; Mp: 123-125° C.

### 3-(4-Benzylcarbamoyl)-7-Oxabicyclo(2.2.1)heptane-2-carboxylic acid (11)

¹H NMR (DMSO-d6): 1.50-1.65 (m, 4H, CH₂), 2.97 (dd, J=26.4 Hz, 2′-1, CH₂), 439 (d, J=3.9 Hz, 2H, 2×CH), 4.61 (d, J=3.9 Hz, 1H, 2×CH), 4.75 (d, J=2.2 Hz, 1H, 2×CH), 5.07 (brs, 1H, OH), 7.19 (d, J=8.3 Hz, 1H, CH—Ar), 7.45 (d, J=8.3 Hz, 1H, CH—Ar), 9.62 (s, 1H, NH); ¹³C NMR (DMSO-d6): 29.3 (CH₂), 29.9 (CH₂), 52.6 (CH), 54.5 (CH), 63.6 (CH₂), 77.8 (CH), 79.6 (CH), 119.9 (2×CH—Ar), 127.7 (2×CH—Ar), 138.0 (C—Ar), 138.7 (C—Ar), 170.1 (CON), 173.1 (COOH); Mp: 137-138° C.

### 3-(Ethylmorpholine carbamoyl)-7-Oxabicyclo(2.2.1)heptane-2-carboxylic acid (12)

¹H NMR (DMSO-d6): 1.41-1.50 (m, 4H, 2×CH₂), 2.34-2.39 (m, 4H, 2×CH₂), 2.77 (brs, 2H, CH₂), 3.09 (d, J=5.1 Hz, 2H), 3.56 (brs, 4H, 2×CH₂), 4.45 (d, J=2.8 Hz, 1H, CH), 4.71 (s, 1H, CH), 7.20 (s, 1H, NH); ¹³C NMR (DMSO-d6): 28.4 (CH₂), 28.6 (CH₂), 52.9 (2×CH₂), 53.7 (CH₂), 56.8 (CH₂), 65.9 (2×CH₂), 77.1 (CH), 78.4 (CH), 171.2 (CON), 172.9 (COOH); Mp: 93-96° C.

### 3-(methyl-3,4 dimethoxyphenyl carbamoyl)-7-Oxabicyclo(2.2.1)heptane-2-carboxylic acid (17)

¹H NMR (DMSO): 7.82 (1H, t J=5.6 Hz), 6.87-6.84 (2H, m), 6.78-6.75 (1H, m), 4.73 (1H, d J=2.8 Hz), 4.49 (1H, d J=3.1 Hz), 4.15 (2H, dq J=16.6 Hz, J=5.9 Hz) 3.72 (3H, s), 3.70 (3H, s) 2.92 (1H, d J=9.7 Hz), 2.83 (1H, d J=9.7 Hz), 1.52-1.43 (4H, m); ¹³C NMR (DMSO): 172.3, 170.3, 148.6, 147.6, 131.8, 119.3, 111.7, 111.2, 78.8, 76.7, 55.5, 55.3, 52.9, 51.3, 41.9, 28.8, 28.3; Mp: 155-156° C.

### 3-(7-Carboxyheptylcarbamoyl)-7-oxabicyclo[2.2.1]heptane-2-carboxylic acid (18)

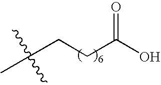

¹H NMR (DMSO-d₆): 1.23 (6H, m), 1.51-1.46 (4H, m), 2.17 (2H, t), 2.81 92H, t), 2.96 (2H, q), 4.45 (1H, d), 4.70 (1H, d), 7.27 (1H, t); ¹³C NMR (DMSO-d₆): 24.4, 26.2, 28.4, 28.7, 28.8, 33.6, 38.4, 51.4, 53.0, 76.7, 78.7, 170.2, 172.3, 174.4; Mp: 125-126° C.

### 3-(Octanoic acid carbamoyl)-7-Oxabicyclo(2.2.1)heptane-2-carboxylic acid (10)

¹H NMR (DMSO): 7.27 (1H, t), 4.70 (1H, d), 4.45 (1H, d), 2.96 (2H, q), 2.81 92H, t), 2.17 (2H, t), 1.51-1.46 (4H, m), 1.23 (6H, m); ¹³C NMR (DMSO): 174.4, 172.3, 170.2, 78.7, 76.7, 53.0, 51.4, 38.4, 33.6, 28.8, 28.7, 28.4, 26.2, 24.4; M.p: 125-126° C.

### 3-(4-hydroxyphenyl)-2-propionic acid)-7-Oxabicyclo(2.2.1)heptane-2,3-dicarboximide (19)

M.W.=331.32, C₁₇H₁₇NO₆; Elemental Analysis: C, 61.63; H, 5.17; N, 4.23; O, 28.97.

### 3-Octadecylcarbamoyl-7-oxabicyclo[2.2.1]heptane-2-carboxylic acid (21)

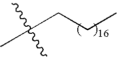

¹H NMR (CDCl₃): 0.87 (3H, t), 1.25 (30H, s), 1.45 (2H, m), 1.55 (2H, m), 1.80 (2H, m), 3.17-3.01 (4H, m), 4.65 (1H, d), 5.00 (1H, d), 6.71 (1H, t); ¹³C NMR (CDCl₃): 13.4, 21.9, 25.7, 26.9, 28.4, 28.5 28.6, 28.8, 28.9, 29.0, 30.7, 38.5, 51.4, 53.0, 76.7, 78.7, 170.3, 172.3; Mp: 124-126° C.

### 3-Allylcarbamoyl-7-oxabicyclo[2.2.1]heptane-2-carboxylic acid (22)

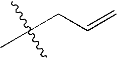

¹H NMR (CDCl₃): 1.46-1.52 (m, 4H), 2.91 (d, J=5.1 Hz, 1H), 2.16 (d, J=5.1 Hz, 1H), 3.72 (m, 2H), 4.67 (t, J=5.2 Hz, 2H), 5.06-5.13 (m, 2H), 5.76-5.82 (m, 1H), 8.14 (brs, 1H); ¹³C NMR (CDCl₃): 24.5, 29.0, 50.0, 51.8, 77.6, 79.6, 115.0, 135.2, 168.9, 173.0, 173.8.

### 3-(3-Carboxpropylcarbamoyl)-7-oxabicyclo[2.2.1]heptane-2-carboxylic acid (23)

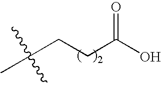

¹H NMR (CDCl₃): 1.52 (4H, m), 2.16 (2H, t), 2.78 (2H, t), 3.02 (2H, m), 4.47 (1H, d), 4.72 (1H, d), 7.25 (1H, t); ¹³C NMR (CDCl₃): 24.8, 28.3, 28.7, 31.7, 37.9, 52.3, 53.3, 76.9, 78.3, 170.9, 172.8, 174.7; Mp: 135-137° C.

### 3-(5-Carboxypentylcarbamoyl)-7-oxabicyclo[2.2.1]heptane-2-carboxylic acid (24)

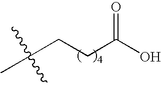

¹H NMR (DMSO-d₆): 1.34 (4H, m), 1.47 (2H, m), 2.16 (2H, t J=7.3 Hz), 2.96 (2H, q J=6.0 Hz), 4.46 (2H, d J=4.0 Hz), 4.71 (1H, d J=3.5 Hz), 7.28 (1H, t); ¹³C NMR (DMSO-d₆): 24.2, 25.9, 28.3, 28.4, 28.6, 38.2, 38.9, 51.6, 53.1, 76.72, 78.6, 170.3, 172.3, 174.5; Mp: 98-100° C.

### 3-(6-Hydroxyhexylcarbamoyl)-7-oxabicyclo[2.2.1]heptane-2-carboxylic acid (26)

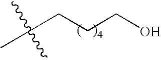

¹H NMR (DMSO-d₆): 1.43 (10H, m), 2.75 (2H, m), 2.94 (2H, quin, J=5.64 Hz), 3.35 (2H, t, J=6.33), 4.43 (1 h, d, J=4.23), 4.67 (1H, d), 7.27 (1H, t, J=5 Hz); ¹³C NMR (DMSO-d₆): 25.1, 25.8, 28.5, 28.7, 28.9, 32.2, 32.4, 38.4, 53.3, 53.54, 60.49, 77.3, 78.4, 171.1, 173.4; Mp: 47-49° C.

### 3-[2-(3H-Imidazol-4-yl)-ethylcarbamoyl]-7-oxa-bicyclo[2.2.1]heptane-2-carboxylic acid (28)

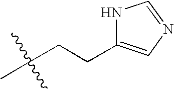

¹H NMR (DMSO-d₆): 1.41-1.46 (4H, m), 2.58 (2H, m), 2.82 (2H, m), 3.18 (2H, m), 4.45 (1H, d), 4.70 (1H, d), 6.77 (1H, t), 7.35 (1H, m), 7.50 (1H, m); ¹³C NMR (DMSO-d₆): 23.9, 27.9, 28.30, 49.3, 51.6, 53.10, 78.27, 78.55, 116.6, 134.7, 143.1, 170.4, 172.4; Mp: 145-147° C.

### 3-(4-Phenylcarbamoyl)-7-Oxabicyclo(2.2.1)heptane-2-carboxylic acid (32)

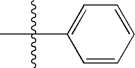

¹H NMR (DMSO-d₆): 1.48-1.59 (m, 4H), 2.92 (d, J=9.4 Hz, 1H), 3.03 (d, J=9.4 Hz, 1H), 4.62 (d, J=3.9 Hz, 4H), 4.76 (s, 1H), 7.01 (t, J=6.7 Hz, 1H), 7.25 (t, J=7.6 Hz, 1H), 7.50 (d, J=7.6 Hz, 1H), 9.64 (s, 1H, NH); ¹³C NMR (DMSO-d₆): 29.3, 29.8, 52.6, 54.4, 77.8, 79.6, 120.1 (2C), 123.8, 129.4 (2C), 140.1, 170.1, 173.1; Mp: 170° C.

### 3-(2-Chlorophenylcarbamoyl)-7-Oxabicyclo(2.2.1)heptane-2-carboxylic acid (33)

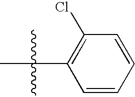

¹H NMR (DMSO-d₆): 1.55-1.66 (m, 4H), 3.12 (dd, J=13.9 and 9.9 Hz, 2H), 4.75 (d, J=4.3 Hz, 1H), 4.88 (s, 1H), 7.08 (t, J=7.6 Hz, 1H), 7.28 (t, J=7.2 Hz, 1H), 7.43 (d, J=7.9 Hz, 1H), 8.03 (d, J=7.9 Hz, 1H), 9.02 (s, 1H); ¹³C NMR (DMSO-d₆): 29.2, 29.4, 52.9, 55.3, 123.7, 124.3, 125.7, 128.3, 130.0, 136.0, 170.6, 173.0; Mp: 135-136° C.

### 3-(3-Chlorophenylcarbamoyl)-7-Oxabicyclo(2.2.1)heptane-2-carboxylic acid (34)

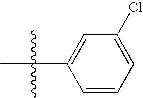

¹H NMR (DMSO-d₆): 1.48-1.64 (m, 4H), 2.94 (d, J=9.6 Hz, 1H), 3.03 (d, J=9.6 Hz, 1H), 4.62 (d, J=3.8 Hz, 1H), 4.76 (s, 1H), 7.06 (dd, 2.0 and 1.6 Hz, 1H), 7.28 (t, J=8.1 Hz, 1H), 7.76 (d, J=1.6 Hz, 1H), 9.86 (s, 1H); ¹³C NMR (DMSO-d₆): 29.3, 29.8, 52.7, 54.3, 77.8, 79.4, 118.4, 119.6, 123.5, 131.1, 133.8, 140.5, 170.6, 173.0; Mp: 163-164° C.

### 3-(4-Bromophenylcarbamoyl)-7-Oxabicyclo(2.2.1)heptane-2-carboxylic acid (35)

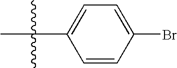

¹H NMR (DMSO-d₆): 1.45-1.60 (4H, m), 2.93 (1H, d J=9.3 Hz), 3.03 (1H, d J=9.6 Hz), 4.63 (1H, d J=3.1 Hz), 4.76 (1H, d J=3.3 Hz), 7.42-7.51 (4H, qJ=8.7 Hz), 9.78 (1H, s), 11.96 (1H, s); ¹³C NMR: (DMSO-d₆): 28.3, 28.9, 51.6, 53.3, 76.8, 78.5, 114.3, 121.0, 131.3, 138.5, 169.4, 172.0; Mp: 187-189° C.

### 3-(3-Iodophenylcarbamoyl)-7-Oxabicyclo(2.2.1)heptane-2-carboxylic acid (36)

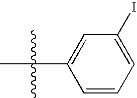

¹H NMR: (DMSO-d₆): 1.48-1.60 (4H, m), 2.91 (1H, d J=9.6 Hz), 3.00 (1H, d J=9.6 Hz), 4.63 (1H, d J=3.9 Hz), 4.76 (1H, d J′=2.7 Hz), 7.10 (1H, t J=8.1 Hz), 7.41-7.34 (2H, m), 8.08 (1H, d J=1.5 Hz), 9.77 (1H, s), 11.96 (1H, s); ¹³C NMR: (DMSO-d₆): 28.3, 28.9, 51.6, 53.3, 76.8, 78.5, 94.3, 118.3, 127.4, 130.5, 131.4, 140.6, 169.5, 172.1; Mp: 192-193° C.

### 3-(4-Iodophenylcarbamoyl)-7-Oxabicyclo(2.2.1)heptane-2-carboxylic acid (37)

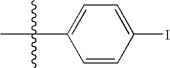

¹H NMR (DMSO-d₆): 1.52-1.56 (m, 4H), 2.92 (d, J=9.6 Hz, 1H), 3.02 (d, J=9.6 Hz, 1H), 4.62 (s, 1H), 4.76 (s, 1H), 7.35 (d, J=8.7 Hz, 1H), 7.59 (d, J=8.7 Hz, 1H), 9.74 (s, 1H), 11.9 (brs, 1H); ¹³C NMR (DMSO-d₆): 28.3, 28.9, 51.6, 53.4, 76.8, 78.5, 86.0, 121.3 (2C), 137.1 (2C), 139.0, 169.4, 172.0; Mp: 184° C.

### 3-(4-Nitrophenylcarbamoyl)-7-Oxabicyclo(2.2.1)heptane-2-carboxylic acid (38)

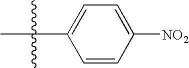

¹H NMR (DMSO-d₆): 1.50-1.58 (m, 4H), 2.99 (d, J=9.3 Hz, 1H), 3.09 (d, J=9.3 Hz, 1H), 4.64 (s, 1H), 4.75 (s, 1H), 7.76 (d, J=8.7 Hz, 2H), 8.17 (d, J=8.7 Hz, 2H), 10.33 (s, 1H), 12.01 (brs, 1H); ¹³C NMR (DMSO-d₆): 29.3, 29.8, 52.8, 54.3, 119.5 (2C), 125.7 (2C), 142.8, 146.4, 171.1, 172.9; Mp: 154° C.

### 3-(2-hydroxyphenylcarbamoyl)-7-Oxabicyclo(2.2.1)heptane-2-carboxylic acid (39)

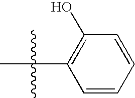

¹H NMR (DMSO-d₆): 1.52-1.65 (m, 4H), 3.04 (d, J=9.9 Hz, 1H), 3.11 (d, J=9.9 Hz, 1H), 4.67 (d, J=4.2 Hz, 1H), 4.84 (s, 1H), 6.71 (t, J=7.8 Hz, 1H), 6.78-6.85 (m, 2H), 7.89 (d, J=7.8 Hz, 1H), 8.88 (s, 1H), 9.65 (brs, 1H), 12.1 (brs, 1H). ¹³C NMR (DMSO-d₆): 28.3, 28.4, 51.9, 54.6, 76.9, 78.9, 114.8, 118.7, 120.3, 123.4, 126.7, 146.6, 169.5, 171.9.

### 3-(4-Hydroxyphenyl carbamoyl)-7-Oxabicyclo(2.2.1)heptane-2-carboxylic acid (40)

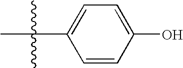

¹H NMR (DMSO-d₆): 1.47-1.56 (m, 4H), 2.89 (d, J=9.6 Hz, 1H), 2.99 (d, J=9.6 Hz, 1H), 4.59 (d, J=3.8 Hz, 1H), 4.75 (s, 1H), 6.63 (d, J=8.7 Hz, 2H), 7.25 (d, J=8.7 Hz, 2H), 9.06 (s, 1H), 9.26 (brs, 1H); ¹³C NMR: (DMSO-d₆): 28.3, 28.9, 51.5, 53.4, 76.7, 78.6, 114.8 (2C), 121.0 (2C), 130.7, 153.1, 168.5, 172.1.

### 3-(4′-Benzoic acid carbamoyl)-7-Oxabicyclo(2.2.1)heptane-2-carboxylic acid (41)

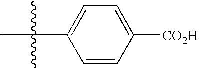

¹H NMR (DMSO-d₆): 1.49-1.60 (4H, m), 2.95 (1H, d J=9.5 Hz), 3.06 (1H, d J=9.5 Hz), 4.63 (1H, d J=3.5 Hz), 4.76 (1H, d J=2.1 Hz), 7.61 (2H, d J=8.5 Hz), 7.85 (2H, d J=8.5 Hz), 9.98 (s, 1H); ¹³C NMR: (DMSO-d₆): 28.3, 28.8, 51.7, 53.4, 76.9, 78.5, 118.3, 124.8, 130.2, 143.2, 166.9, 169.8, 172.0; Mp: 271-273° C.

### 3-(4′-Methoxyphenyl carbamoyl)-7-Oxabicyclo(2.2.1)heptane-2-carboxylic acid (42)

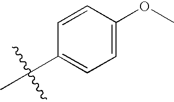

¹H NMR (DMSO-d₆): 1.51-1.56 (m, 4H), 2.90 (d, J=9.6 Hz, 1H), 3.09 (d, J=9.6 Hz, 1H), 3.69 (s, 1H), 4.60 (d, J=3.7 Hz, 1H), 4.76 (d, J=3.7 Hz, 1H), 6.83 (d, J=8.9 Hz, 2H), 7.40 (d, J=8.9 Hz, 2H), 9.39 (s, 1H); ¹³C NMR (DMSO-d₆): 29.3, 29.8, 52.5, 54.4, 56.1, 77.8, 79.6, 114.6, 121.7, 133.3, 156.0, 169.7, 173.1; HRMS Calculated 292.1140, Found 292.1140.

### 3-(4′-Methylthio benzene carbamoyl)-7-Oxabicyclo(2.2.1)heptane-2-carboxylic acid (43)

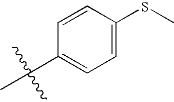

¹H NMR (DMSO-d₆): 1.48-1.60 (m, 4H), 2.42 (s, 3H), 2.92 (d, J=9.6 Hz, 1H), 3.03 (d, J=9.6 Hz, 1H), 4.62 (d, J=3.9 Hz, 1H), 4.84 (d, J=3.9 Hz, 1H), 7.19 (d, J=8.4 Hz, 2H), 7.47 (d, J=8.4 Hz, 2H), 9.61 (s, 1H), 11.9 (s, 1H); ¹³C NMR (DMSO-d₆): 16.7, 29.3, 29.8, 52.6, 54.3, 77.7, 79.5, 120.8 (2C), 128.1 (2C), 132.2, 137.8, 170.1, 173.0; HRMS Calculated 308.0912, Found 308.0947.

### 3-(2′-Methylthio benzene carbamoyl)-7-Oxabicyclo(2.2.1)heptane-2-carboxylic acid (43)

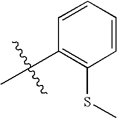

¹H NMR (DMSO-d₆): 1.57 (m, 4H), 2.37 (s, 3H), 3.09 (s, 2H), 4.75 (d, J=4.2 Hz, 1H), 4.88 (s, 1H), 7.08 (t, J=7.5 Hz, 2H), 7.19 (t, J=7.5 Hz, 1H), 7.38 (d, J=7.8 Hz, 1H), 7.84 (d, J=7.8 Hz, 1H), 8.98 (s, 1H), 12.2 (s, 1H); ¹³C NMR (DMSO-d₆): 16.5, 28.3, 28.4, 51.8, 54.6, 77.0, 78.9, 122.2, 124.5, 126.6, 128.2, 129.7, 137.1, 169.6, 172.0; HRMS Calculated: 308.0912, Found: 308.0951.

### 3-(4′-Pentyloxy benzene carbamoyl)-7-Oxabicyclo(2.2.1)heptane-2-carboxylic acid (45)

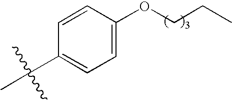

¹H NMR (DMSO-d₆): 0.88 (t, J=6.9 Hz, 3H), 1.34-1.36 (m, 4H), 1.51-1.67 (m, 6H), 2.91 (d, J=9.6 Hz, 1H), 3.01 (d, J=9.6 Hz, 1H), 3.89 (t, J=6.5 Hz, 2H), 4.61 (d, J=3.8 Hz, 1H), 4.84 (d, J=3.8 Hz, 1H), 6.82 (d, J=8.9 Hz, 2H), 7.39 (d, J=8.9 Hz, 1H), 9.41 (s, 1H), 10.85 (brs, 1H); ¹³C NMR (DMSO-d₆): 14.8, 22.8, 28.6, 29.3 (2C), 29.8, 52.5, 54.3, 68.5, 77.7, 79.6, 115.2 (2C), 121.7 (2C), 133.2, 155.4, 169.7, 173.1; HRMS Calculated 348.1800, Found 348.1821; Mp: 156° C.

### 3-(4′-Octylloxy benzene carbamoyl)-7-Oxabicyclo(2.2.1)heptane-2-carboxylic acid (46)

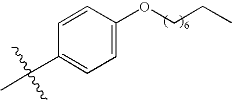

¹H NMR (DMSO-d₆): 0.82 (t, J=7.0 Hz, 3H), 1.24 (brs, 8H), 1.45-1.70 (m, 4H), 1.75 (qn, J=3.0 Hz, 3H), 2.95, 3.58 (qn, J=2.5 Hz, 4H), 3.86 (t, J=6.5 Hz, 2H), 4.59 (d, J=4.0 Hz, 1H), 4.75 (d, J=2.7 Hz, 1H), 6.81 (d, J=9.0 Hz, 1H), 7.38 (d, J=9.0 Hz, 1H), 9.49 (s, 1H); ¹³C NMR (DMSO-d₆): 14.8, 22.9, 26.0, 26.4, 29.5, 29.6, 32.1, 52.5, 54.3, 67.9, 68.5, 77.7, 79.6, 115.3, 121.7, 133.2, 155.4, 169.7, 173.1; HRMS Calculated 390.2236, Found 390.2279; Mp: 132° C.

### 3-(2′-Benzyl alcohol carbamoyl)-7-Oxabicyclo(2.2.1)heptane-2-carboxylic acid (47)

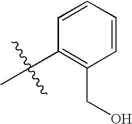

¹H NMR (DMSO-d₆): 1.51-1.62 (m, 4H), 3.03 (t, J=10.3 Hz, 2H), 4.45 (s, 2H), 4.69 (d, J=4.2 Hz, 1H), 4.08 (s, 1H), 5.22 (brs, 1H), 7.06 (t, J=7.1 Hz, 1H), 7.19 (t, J=7.1 Hz, 1H), 7.32 (d, J=7.4 Hz, 1H), 7.63 (d, J=7.9 Hz, 1H), 8.99 (s, 1H); ¹³C NMR (DMSO-d₆): 29.0, 29.1, 52.4, 54.7, 60.7, 77.6, 79.3, 123.5, 124.4, 127.4, 127.9, 133.9, 136.4, 169.9, 172.7; Mp: 145-146° C.; HRMS Calculated 292.1140, Found 292.1175.

### 3-(4′-Benzyl alcohol carbamoyl)-7-Oxabicyclo(2.2.1)heptane-2-carboxylic acid (48)

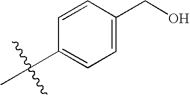

¹H NMR (DMSO-d₆): 1.50-1.65 (m, 4H), 2.97 (dd, J=26.4 Hz, 2H), 4.39 (d, J=3.9 Hz, 2H), 4.61 (d, J=3.9 Hz, 1H), 4.75 (d, J=2.2 Hz, 1H), 5.07 (brs, 1H), 7.19 (d, J=8.3. Hz, 1H), 7.45 (d, J=8.3 Hz, 1H), 9.62 (s, 1H); ¹³C NMR (DMSO-d₆): 29.3, 29.9, 52.6, 54.5, 63.6, 77.8, 79.6, 119.9 (2C), 127.7 (2C), 138.0, 138.7, 170.1, 173.1; HRMS Calculated 292.1140, Found 292.1179; Mp: 137-138° C.

### 3-(3′-Benzyl alcohol carbamoyl)-7-Oxabicyclo(2.2.1)heptane-2-carboxylic acid (49)

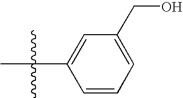

¹H NMR (DMSO-d₆): 1.49-1.60 (m, 4H), 2.92 (d, J=9.6 Hz, 1H), 3.05 (d, J=9.6 Hz, 1H), 4.44 (s, 2H), 4.62 (d, J=3.8 Hz, 1H), 4.75 (d, J=3.8 Hz, 1H), 5.11 (brs, 1H), 7.76 (d, J=7.4 Hz, 1H), 7.19 (t, J=7.7 Hz, 1H), 7.37 (d, J=8.0 Hz, 1H), 7.49 (s, 1H), 9.54 (s, 1H), 10.9 (brs, 1H); ¹³C NMR (DMSO-d₆): 29.3, 29.8, 52.5, 54.5, 63.7, 77.8, 79.6, 118.2, 118.4, 121.9, 129.1, 140.0, 143.9, 170.1, 173.1; HRMS Calculated 292.1140, Found 292.1175.

### 3-(4′-Ethylene benzene carbamoyl)-7-Oxabicyclo(2.2.1)heptane-2-carboxylic acid (50)

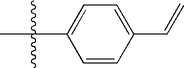

¹H NMR (DMSO-d₆): 1.48-1.60 (m, 4H), 2.92 (d, J=9.6 Hz, 1H), 3.04 (d, J=9.6 Hz, 1H), 4.62 (d, J=3.8 Hz, 1H), 4.76 (s, 1H), 5.13 (d, J=11.1 Hz, 1H), 5.68 (d, J=17.7 Hz, 1H), 6.85 (dd, J=17.7 and 11.1 Hz), 7.36 (d, J=8.5 Hz, 1H), 7.49 (d, J=8.5 Hz, 1H), 9.68 (s, 1H); ¹³C NMR (DMSO-d₆): 29.2, 29.6, 52.6, 54.4, 77.7, 79.3, 113.2, 120.0 (2C), 127.1 (2C), 132.8, 137.0, 139.7, 169.9, 172.7; HRMS Calculated 288.1191, Found 288.1244; Mp: 157° C.

### 3-(3′-Ethynyl benzene carbamoyl)-7-Oxabicyclo(2.2.1)heptane-2-carboxylic acid (51)

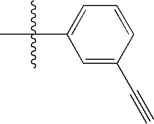

¹H NMR (DMSO-d₆): 1.52-1.54 (m, 4H), 2.93 (d, J=9.6 Hz, 2H), 3.03 (d, J=9.6 Hz, 2H), 4.10 (s, 1H), 4.63 (d, J=3.8, 1H), 4.76 (s, 1H), 7.11 (d, J=7.3 Hz, 1H), 7.27 (t, J=7.8 Hz, 1H), 7.43 (d, J=8.1 Hz, 1H), 7.73 (s, 1H), 9.75 (s, 1H), 10.9 (brs, 1H); ¹³C NMR (DMSO-d₆): 28.3, 28.8, 51.8, 53.5, 77.0, 78.4, 79.4, 83.3, 119.9, 121.8, 122.3, 126.1, 128.8, 139.3, 169.4, 171.9; Mp: 166° C.

### 3-(4′-Benzene acetic acid carbamoyl)-7-Oxabicyclo(2.2.1)heptane-2-carboxylic acid (52)

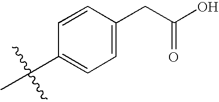

¹H NMR (DMSO-d₆): 1.49-1.57 (4H, m), 2.92 (1H, d J=9.6 Hz), 3.03 (1H, d J=9.6 Hz), 3.47 (2H, s), 4.62 (1H, a J=4.2 Hz), 4.76 (1H, d J=3.1 Hz), 7.13 (2H, d J=8.4 Hz), 7.44 (2H, d J=8.7 Hz), 9.55 (1H, NH), 12.04 (1H, COOH); ¹³C NMR (DMSO-d₆): 28.3, 28.9, 51.6, 53.4, 76.8, 78.6, 119.0, 129.3, 129.4, 137.7, 169.1, 172.1, 172.7; Mp: 268-270° C.

### 3-(3′-Benzene acetic acid carbamoyl)-7-Oxabicyclo(2.2.1)heptane-2-carboxylic acid (53)

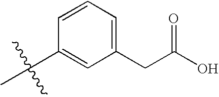

¹H NMR (DMSO-d₆): 1.49-1.57 (4H, t), 2.91 (2H, d J=9.6 Hz), 3.05 (2H, d J=9.6 Hz), 3.49 (2H, s), 4.61 (1H, d J=3.9 Hz), 4.76 (1H, d J=3.0 Hz), 6.89 (1H, d J=7.5 Hz), 7.19 (1H, t J=7.5 Hz), 7.39 (1H, d J=8.1 Hz), 7.46 (1H, s), 9.63 (1H,s NH), 11.99 (1H, COOH); ¹³C NMR (DMSO-d₆): 28.4, 28.9, 40.8, 51.5, 53.5, 76.8, 78.7, 117.4, 119.8, 123.9, 128.4, 135.3, 139.2, 169.2, 172.1, 172.4; Mp: 144-146° C.

### 3-(4′-Benzene propanoic acid carbamoyl)-7-Oxabicyclo(2.2.1)heptane-2-carboxylic acid (54)

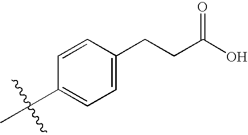

¹H NMR (DMSO-d₆): 1.52-1.56 (4H, m), 2.49 (2H, t), 2.74 (2H, t), 2.93 (1H, d), 3.01 (1H, d), 4.61 (1H, 4.2 Hz), 4.76 (1H, 3.3 Hz), 7.10 (2H, d J=8.4 Hz), 7.39 (2H, d J=8.4 Hz), 9.50 (1H, s NH), 11.97 (2H, COOH); ¹³C NMR (DMSO-d₆): 28.3, 28.8, 29.7, 35.3, 51.6, 53.5, 76.8, 78.6, 119.2, 128.2, 135.4, 137.1, 169.0, 172.1, 173.6; Mp: 189-191° C.

### 3-(4′-Morpholino-benzene carbamoyl)-7-Oxabicyclo(2.2.1)heptane-2-carboxylic acid (55)

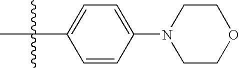

¹H NMR (DMSO-d₆): 1.47-1.56 (m, 4H), 2.88-3.02 (m, 6H), 4.60 (d, J=3.7 Hz, 1H), 4.76 (d, J=2.1 Hz, 1H), 6.85 (d, 8.9 Hz, 1H), 7.36 (d, J=8.9 Hz, 1H), 9.35 (s, 1H), 10.9 (brs, 1H); ¹³C NMR (DMSO-d₆): 29.3, 29.8, 50.0, 52.5, 54.4, 67.0, 77.7, 79.6 (CH), 116.3 (2C), 121.2 (2C), 132.5, 148.0, 169.6, 173.1; HRMS Calculated 347.1562, Found 347.1614.

### 3-(2′-Ethyl-phenylcarbamoyl)-7-oxa-bicyclo[2.2.1]heptane-2-carboxylic acid (57)

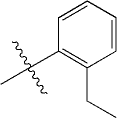

¹H NMR (DMSO-d₆): 1.23 (3H, t J=7.6 Hz), 1.53-1.64 (4H, m), 2.54 (2H, q J=7.5 Hz), 3.07 (2H, q J=5.8 Hz), 4.71 (1H, d J=4.32 Hz), 4.85 (1H, d J=4.12 Hz), 7.02-7.19 (3H, m), 7.65 (1H, d J=7.6 Hz), 8.70 (1H, NH), 11.85 (1H, COOH); ¹³C ¹H NMR (DMSO-d₆): 14.0, 23.5, 28.4, 51.7, 54.1, 77.0, 79.0, 123.2, 124.4, 125.8, 128.2, 135.1, 135.7, 169.3, 172; Mp: 171-172° C.

### 3-(2′,6′-Dimethylphenylcarbamoy07-oxa-bicyclo[2.2.1]heptane-2-carboxylic acid (58)

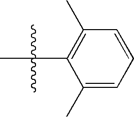

¹H NMR (DMSO-d₆): 1.52-1.59 (4H, m), 2.13 (6H, s), 2.90 (1H, d J=9.6 Hz), 3.15 (1H, d J=9.6 Hz), 4.60 (1H, d J=4.7 Hz), 4.78 (1H, d J=3.6 Hz), 7.02 (3H, s), 8.96 (1H, NH), 11.86 (1H, COOH); ¹³C NMR (DMSO-d₆): 18.1, 28.5, 29.0, 51.0, 52.9, 76.6, 79.3, 126.1, 127.4, 135.1, 135.2, 168.8, 172.2; Mp: 193-195° C.

### 3-(2′,4′-Dimethylphenylcarbamoyl)-7-oxa-bicyclo[2.2.1]heptane-2-carboxylic acid (59)

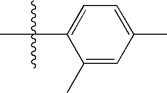

¹H NMR (DMSO-d₆): 1.52-1.68 (m, 4H), 2.12 (s, 3H), 2.29 (s, 3H), 3.01 (d, J=9.7 Hz, 1H), 3.07 (d, J=9.7 Hz, 1H), 4.69 (d, J=3.7 Hz, 1H), 4.82 (s, 1H), 6.92 (m, 2H), 7.46 (d, J=8.0 Hz, 1H), 8.63 (s, 1H); ¹³C NMR (DMSO-d₆): 17.2, 20.3, 28.3, 28.5, 51.7, 53.9, 77.0, 78.9, 123.0, 126.2, 129.4, 130.5, 133.1, 133.9, 169.0, 172.2; HRMS Calculated 290.1348, Found 290.1400.

### 3-(2′,3′-Dimethylphenylcarbamoyl)-7-oxa-bicyclo[2.2.1]heptane-2-carboxylic acid (60)

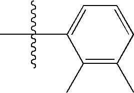

¹H NMR (DMSO-d₆): 1.52-1.59 (m, 4H), 2.05 (s, 3H), 2.22 (s, 3H), 3.01 (d, J=9.7 Hz, 1H), 3.08 (d, J=9.7 Hz, 1H), 4.69 (d, J=4.2 Hz, 1H), 4.81 (s, 1H), 6.92 (d, 5 J=7.2 Hz, 1H), 7.00 (t, J=7.7 Hz, 1H), 7.34 (d, J=7.7 Hz, 1H), 8.78 (s, 1H), 11.1 (brs, 1H); ¹³C NMR (DMSO-d₆): 14.3, 21.1, 29.3, 29.6, 52.7, 54.7, 77.9, 79.9, 122.7, 125.9, 126.0, 129.9, 137.2, 137.4, 170.1, 173.2; Mp: 177° C.; HRMS Calculated 290.1390, Found 290.1392.

### 3-(2′,4′,6′-Trimethylphenylcarbamoyl)-7-oxa-bicyclo[2.2.1]heptane-2-carboxylic acid (61)

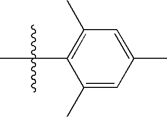

¹H NMR (DMSO-d₆): 1.51-1.66 (m, 4H), 2.08 (s, 6H), 2.19 (s, 3H), 2.88 (d, J=9.4 Hz, 1H), 3.12 (d, J=9.4 Hz, 1H), 4.58 (d, J=4.5 Hz, 1H), 4.77 (s, 1H), 6.82 (s, 2H), 8.87 (s, 1H), 11.85 (brs, 1H); ¹³C NMR (DMSO-d₆): 17.9 (2C), 18.0, 28.5, 29.0, 50.9, 52.9, 76.6, 79.3, 128.0, 132.4, 134.9, 135.0, 168.8, 172.2; HRMS Calculated 304.1504, Found 304.1551.

### 3-(3′,5′-Di-tert-butylphenylcarbamoyl)-7-oxa-bicyclo[2.2.1]heptane-2-carboxylic acid (62)

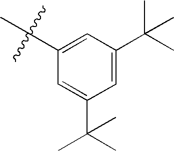

¹H NMR (DMSO-d₆): 1.25 (s, 18H), 1.49-1.62 (m, 4H), 2.89 (d, J=9.5 Hz, 2H), 3.04 (d, J=9.5 Hz, 2H), 4.61 (d, J=4.0 Hz, 1H), 4.84 (s, 1H), 7.05 (s, 1H), 7.39 (s, 2H), 9.50 (s, 1H), 10.9 (brs, 1H); ¹³C NMR (DMSO-d₆): 29.4, 29.8, 32.1 (6C), 35.3, 52.3, 54.6, 77.8, 79.8, 114.3, 117.5, 139.6, 151.4, 169.9 (CON), 173.2; Mp: 182° C.; HRMS Calculated 374.2253, Found 374.2339.

### 3-(2′-Naphthylcarbamoyl)-7-oxa-bicyclo[2.2.1]heptane-2-carboxylic acid (63)

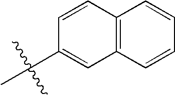

¹H NMR (DMSO-d₆): 1.57-1.68 (m, 4H), 3.09 (d, J=9.6 Hz, 2H), 3.27 (d, J=9.6 Hz, 2H), 4.81 (d, J=3.8 Hz, 1H), 4.87 (s, 1H), 7.46-7.54 (m, 3H), 7.69 (m, 1H), 7.92 (m, 1H), 7.90 (m, 1H), 8.02 (m, 1H), 9.49 (m, 1H); ¹³C NMR (DMSO-d₆): 29.4, 29.6, 52.8, 54.7, 77.1, 79.9, 120.9, 123.0, 125.4, 126.4, 126.7, 126.8, 127.9, 129.0, 134.4, 134.5, 170.7, 173.2; HRMS Calculated 312.1191, Found 312.1237; Mp: 178-179° C.

### Compound (64)

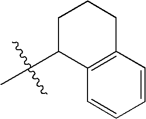

¹H NMR (DMSO-d₆): 2 diasteroisomers; 1.45-1.83 (m, 20H), 2.68 (q, J=6.7 Hz, 4H), 2.82-2.89 (m, 4H), 3.51 (brs, 1H), 4.13 (brs, 1H), 4.51 (dd, J=13.9 and 4.1 Hz, 2H), 4.71 (brs, 2H), 7.05-7.23 (m, 10H), 7.48 (d, J=7.8 Hz, 1H), 7.73 (d, J=7.8 Hz, 1H); ¹³C NMR (DMSO-d₆): 19.6, 20.7, 20.9, 26.5, 29.4, 29.6, 29.7, 30.5, 47.1, 47.2, 52.9, 53.0, 53.7, 54.0, 67.9, 77.8, 77.9, 79.5, 78.8, 126.7, 127.4, 128.0, 129.2, 129.4, 129.5, 129.7, 137.8, 137.9, 138.4, 138.5, 171.0 and 171.1, 173.4 and 173.5; Mp: 150° C.

### 3-[(Pyridin-2-ylmethyl)-carbamoyl]-7-oxa-bicyclo[2.2.1]heptane-2-carboxylic acid (65)

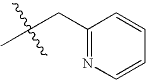

¹H NMR (CDCl₃): 1.45-1.56 (4H, m), 2.87 (1H, d), 2.96 (1H, d), 4.30 (2H, dq), 4.54 (1H, d), 4.73 (1H, d), 7.22 (1H, t), 7.32 (1H, m), 7.71 (1H, m), 8.03 (1H, m), 8.45 (1H, m); ¹³C NMR (CDCl₃): 28.7, 28.9, 53.1, 53.4, 76.9, 78.7, 79.8, 120.9, 122.1, 136.6, 148.5, 158.4, 171.0, 172.6; Mp: 153-155° C.

### 3-(Benzylcarbamoyl)-7-oxa-bicyclo[2.2.1]heptane-2-carboxylic acid (66)

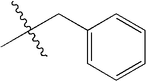

¹H NMR (DMSO-d₆): 1.43-1.56 (4H, m), 2.84 (1H, d J=9.6 Hz), 2.92 (1H, d J=9.7 Hz), 4.21 (2H, sept J=6.1 Hz), 4.50 (1H, d J=2.4 Hz), 4.74 (1H, d J=2.4 Hz), 7.29-7.21 (5H, m), 7.93 (1H, t J=5.7 Hz); ¹³C NMR (DMSO-d₆): 28.3, 28.85, 42.1, 51.2, 52.9, 76.7, 78.7, 126.6, 127.1, 128.1, 139.4, 170.5, 172.3; Mp: 162-163° C.

### 3-(4′-Methoxy-benzylcarbamoyl-7-oxa-bicyclo[2.2.1]heptane-2-carboxylic acid (67)

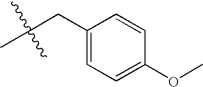

¹H NMR (DMSO-d₆): 1.42-1.52 (4H, m), 2.84, (1H,d J=9.1 Hz), 2.89 (1H, d J=8.9 Hz), 3.71 (3H, s), 4.13 (2H, sept J=6.5 Hz), 4.47 (1H, d J=3.0 Hz), 4.73 (1H, d J=2.9 Hz), 6.85 (2H, d J=8.6 Hz), 7.16 (2H, d J=8.5 Hz), 7.86 (1H, t J=5.6 Hz). ¹³C NMR (DMSO-d₆): 28.3, 28.8, 41.6, 51.2, 52.9, 55.0, 76.7, 78.8, 113.6, 128.5, 131.3, 158.1, 168.9, 172.3; Mp: 144-145° C.

### 3-(3′,4′-Dimethoxy-benzylcarbamoyl)-7-oxa-bicylco[2.2.1]heptane-2-carboxylic acid (68)

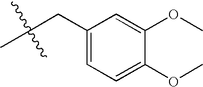

¹H NMR (DMSO-d₆): 1.43-1.52 (4H, m), 2.83 (1H, d J=9.7 Hz), 2.92 (1H, dJ 9.7 Hz), 3.70 (3H, s), 3.72 (3H, s), 4.15 (2H, dq J=16.6 Hz, J=5.9 Hz), 4.49 (1H, d J=3.1 Hz), 4.73 (1H, d J=2.8 Hz), 6.75-6.78 (1H, m), 6.84-6.87 (2H, m), 7.82 (1H, t J=5.6 Hz); ¹³C NMR (DMSO-d₆): 28.3, 28.8, 41.9, 51.3, 52.9, 55.3, 55.5, 76.7, 78.8, 111.2, 111.7, 119.3, 131.8, 147.6, 148.6, 170.3, 172.3; Mp: 155-156° C.

### 3-(4′-Chlorobenzylcaramoyl-7-oxa-bicyclo[2.2.1]heptane-2-carboxylic acid (69)

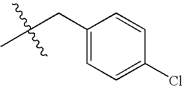

¹H NMR (DMSO-d₆): 1.41-1.56 (4H, m), 2.87 (2H, q J=9.0 Hz), 4.19 (2H, m), 4.50 (1H, d J=4.0 Hz), 4.73 (1H, d J=2.8 Hz), 7.25-7.41 (4H, q), 7.96 (1H, t J=5.6 Hz); ¹³C NMR (DMSO-d₆): 28.4, 28.8, 41.4, 51.4, 52.9, 76.8, 78.7, 128.0, 129.0, 138.6, 170.6, 172.3; Mp: 183-185° C.

### 3-(Phenethylcarbamoyl)-7-oxa-bicyclo[2.2.1]heptane-2-carboxylic acid (70)

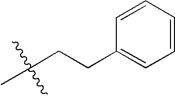

¹H NMR (DMSO-d₆): 1.51 (4H, m), 2.67 (2H, t), 2.82 (2H, s), 3.19 (2H, q), 4.41 (1H, d), 4.72 (1H, d), 7.26-7.17 (5H, m), 7.40 (1H, t NH); ¹³C NMR (DMSO-d₆): 28.4, 28.7, 35.0, 40.12, 51.7, 53.1, 76.8, 78.6, 125.9, 128.2, 128.5, 139.5, 170.5, 172.4; Mp: 144-146° C.

### 3-(Phenyl-propylcarbamoyl)-7-oxa-bicyclo[2.2.1]heptane-2-carboxylic acid (71)

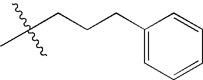

¹H NMR (DMSO-d₆): 1.43-1.56 (4H, m), 1.64 (2H, quin J=7.3 Hz), 2.52 (2H, t J=7.5 Hz), 2.98 (2H, q J=6.2 Hz), 4.47 (1H, d J=4.3 Hz), 4.71 (1H, d J=2.3 Hz), 7.15-7.28 (5H, m), 7.34 (1H, t J=5.3 Hz); ¹³C NMR (DMSO-d₆): 28.6, 28.8, 30.7, 32.5, 38.9, 51.5, 53.0, 76.7, 78.6, 125.6, 128.1, 128.2, 141.7, 170.3, 172.2; Mp: 135-137° C.

### 3-(Phenyl-butylcarbamoyl)-7-oxa-bicyclo[2.2.1]heptane-2-carboxylic acid (72)

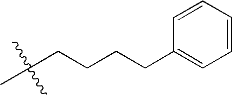

¹H NMR (DMSO-d₆): 1.35-1.60 (8H, m), 2.51 (2H, quin), 2.98 (2H, q), 4.43 91H, d), 4.69 (1H, d), 7.15-7.29 (5H, m), 7.30 (1H, t); ¹³C NMR (DMSO-d₆): 28.3, 28.5, 34.7, 38.2, 49.3, 52.3, 53.3, 77.0, 78.5, 125.6, 128.2, 142.1, 170.7, 172.7; Mp: 105-107° C.

### 3-(4′-Carboxybenzylcarbamoyl)-7-oxa-bicyclo[2.2.1]heptane-2-carboxylic acid (73)

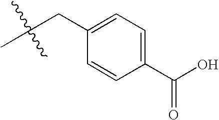

¹H NMR (DMSO-d₆): 1.42-1.61 (4H, m), 2.89 (2H q J=11.7 Hz), 4.26 (2H, dq J=11.7 Hz, J=4.8 Hz), 4.52 (1H, d J=4.2 Hz), 4.73 (1H, d J=2.3 Hz), 7.34 (2H, d J=8.2 Hz), 7.85 (2H, d J=8.2 Hz), 8.02 (1H, t, NH); ¹³C NMR (DMSO-d₆): 28.4, 28.8, 41.9, 51.5, 52.9, 76.8, 78.7, 126.9, 128.1, 129.1, 144.3, 167.4, 170.7, 172.3. M.p 215-217° C.

### Coumpound (74)

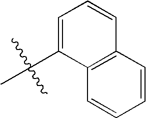

¹H NMR (DMSO-d₆): 1.51-1.62 (m, 4H), 2.96 (d, J=9.9 Hz, 2H), 3.17 (d, J=9.9 Hz, 2H), 4.67 (s, 1H), 4.79 (s, 1H), 7.36-7.53 (m, 3H), 7.74-7.83 (s, 3H), 9.82 (s, 1H), 10.95 (brs, 1H); ¹³C NMR (DMSO-d₆): 29.3, 29.8, 52.7, 54.5, 77.8, 79.6, 116.0, 120.9, 125.3, 127.2, 128.0, 128.3, 129.0, 130.5, 134.3, 137.7, 170.5, 173.1. MP: 237-238° C.

### 3-(1-benzylpiperidin-4-ylcarbamoyl]-7-oxabicyclo[2.2.1]heptane-2-carboxylic acid (75)

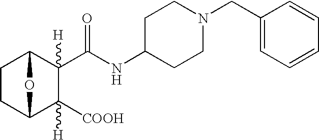

Purification afforded (75) (0.99 g, 87%) as a white crystalline solid. Mp 124-125° C.; ¹H NMR (300 MHz, DMSO-d₆) δ 12.56 (bs, 1H), 7.44-7.39 (m, 5H), 4.90 (d, J=4.5 Hz, 1H), 4.58 (d, J=4.5 Hz, 1H), 4.00 (s, 2H), 3.81-3.72 (m, 1H), 3.35 (s, 1 H), 3.26-3.12 (m, 2H), 2.95 (d, J=9.9 Hz, 1H), 2.90 (d, J=9.9 Hz, 1H), 2.79-2.69 (m, 2H), 2.01-1.94 (m, 2H), 1.75-1.67 (m, 4H), 1.56-1.52 (m, 2H); ¹³C NMR (75 MHz, DMSO-d₆) δ 175.0, 172.6, 131.6, 129.9, 128.1, 127.9, 78.7, 77.8, 60.1, 54.3, 53.8, 50.0, 49.8, 43.7, 28.2, 27.9; Calculated for C₂₀H₂₆N₂O₄; Exact Mass: 358.1893; m/e: 358.1893 (100.0%), 359.1926 (22.2%), 360.1960 (2.4%); Elemental C, 67.02; H, 7.31; N, 7.82; O, 17.85

### 3-(4-Carboxy-3-chlorophenylcarbamoyl)-7-oxabicyclo[2.2.1]heptane-2-carboxylic acid (76)

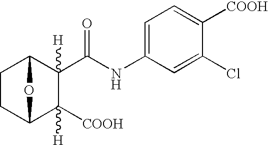

Purification afforded (76) (0.17 g, 17%) as a white crystalline solid. Mp 216-217° C.; ¹H NMR (300 MHz, DMSO-d₆) δ 12.56 (bs, 1H), 10.17 (s, 1H), 7.87 (d, J=1.9 Hz, 1H), 7.80 (d, J=8.6 Hz, 1H), 7.42 (dd, J=8.6, 2.0 Hz, 1H), 4.76 (d, J=2.8 Hz, 1H), 4.65 (d, J=3.6 Hz, 1H); 3.36 (bs, 1H), 3.04 (d, J=9.6 Hz, 1H), 2.96 (d, J=9.6 Hz, 1H), 1.48-1.56 (m, 4H); ¹³C NMR (75 MHz, DMSO-d₆) δ 172.0, 170.1, 165.8, 142.8, 133.0, 132.2, 124.0, 120.1, 116.8, 78.4, 76.9, 53.2, 51.7, 28.8, 28.3; Calculated for C₁₅H₁₄ClNO₆; Exact Mass: 339.0510; m/e: 339.0510 (100.0%), 341.0480 (32.0%), 340.0543 (16.7%), 342.0514 (5.3%), 341.0577 (1.3%), 341.0552 (1.2%); Elemental C, 53.03; H, 4.15; Cl, 10.44; N, 4.12; O, 28.26

### 3-[(1-Methyl-1H-indol-4-yl)methylcarbamoyl]-7-oxabicyclo[2.2.1]heptane-2-carboxylic acid (80)

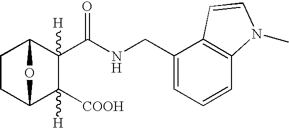

Purification afforded (80) (0.85 g, 76%) as a white crystalline solid. Mp 161° C.; ¹H NMR (300 MHz) (DMSO-d₆) δ 12.08 (bs, 1H), 7.88 (t, J=5.2 Hz, 1H), 7.32 (d, J=7.2 Hz, 1H), 7.29 (d, J=2.9 Hz, 1H), 7.08 (t, J=8.6 Hz, 1H), 6.94 (d, J=7.2 Hz, 1H), 6.48 (d, J=2.9 Hz, 1H), 4.74 (d, J=5.2 Hz, 1H), 4.45 (m, 3H), 3.77 (s, 3H), 2.65 (d, J=9.6 Hz, 1H), 2.83 (d, J=9.6 Hz, 1H), 1.51-1.44 (m, 4H); ¹³C NMR (75 MHz, DMSO-d₆) δ 172.4, 170.2, 136.2, 130.3, 129.2, 126.5, 120.8, 117.6, 108.5, 98.6, 79.0, 76.7, 52.9, 51.1, 40.5, 32.5, 28.8, 28.3; Calculated for C₁₈H₂₀N₂O₄; Exact Mass: 328.1423; m/e: 328.1423 (100.0%), 329.1457 (20.0%), 330.1490 (1.9%); Elemental C, 65.84; H, 6.14; N, 8.53; O, 19.49.

### 3,[(6,7-Dihydro-4H-thieno[3,2-c]pyran-4-yl)methylcarbamoyl]-7-oxabicyclo[2.2.1]heptane-2-carboxylic acid (81)

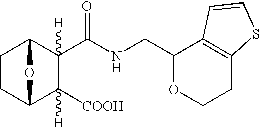

Purification afforded (81) (0.66 g, 56%) as a white crystalline solid. Mp 159-160° C.; ¹H NMR (300 MHz) (DMSO-d₆) δ 11.78 (bs, 1H), 7.64 (t, J=5.6 Hz, 1H), 7.31 (d, J=5.2 Hz, 1H), 6.86 (d, J=5.2 Hz, 1H), 4.71 (d, J=2.9 Hz, 1H), 4.60-4.58 (m, 1H), 4.33 (d, J=3.7 Hz, 1H), 4.11-4.04 (m, 1H), 3.70-3.54 (m, 2H), 3.12-3.03 (m, 1H), 2.91 (d, J=9.7 Hz, 1H), 2.81 (d, J=9.6 Hz, 1H), 2.74 (bs, 1H), 2.48 (dd, J=5.3, 1.7 Hz), 1.51-1.44 (m, 4H); ¹³C NMR (75 MHz, DMSO-d₆) δ 172.3, 170.7, 134.7, 133.3, 123.9, 123.0, 78.8, 76.6, 74.0, 62.3, 52.7, 51.2, 42.6, 28.8, 28.3, 24.9; Calculated for C₁₆H₁₉NO₅S; Exact Mass: 337.0984; m/e: 337.0984 (100.0%), 338.1017 (17.8%), 339.0942 (4.4%), 339.1051 (1.5%), 339.1026 (1.0%); Elemental C, 56.96; H, 5.68; N, 4.15; O, 23.71; S, 9.50.

### 3-(Benzo[d]thiazol-5-ylcarbamoyl)-7-oxabicyclo[2.2.1]heptane-2-carboxylic acid (82)

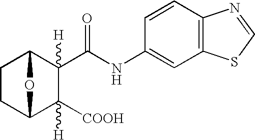

Purification afforded (82) (0.96 g, 88%) as a white crystalline solid. Mp 159-160° C.; ¹H NMR (300 MHz) (DMSO-d₆) δ 12.00 (bs, 1H), 10.0 (bs, 1H), 9.23 (s, 1H), 8.47 (d, J=2.6 Hz, 1H), 7.98 (d, J=8.8 Hz, 1H), 7.52 (dd, J=8.8, 2.6 Hz, 1H), 4.79 (d, J=2.6 Hz, 1H), 4.67 (d, J=2.9 Hz, 1H), 3.11 (d, J=9.6 Hz, 1H), 2.98 (d, J=9.8 Hz, 1H), 1.61-1.49 (m, 4H); ¹³C NMR (75 MHz, DMSO-d₆) δ 172.1, 169.6, 154.3, 148.8, 136.9, 134.1, 122.7, 118.7, 111.4, 78.6, 76.8, 53.3, 51.7, 28.9, 28.3; Calculated for C₁₅H₁₄N₂O₄S; Exact Mass: 318.0674; m/e: 318.0674 (100.0%), 319.0708 (16.7%), 320.0632 (4.4%), 320.0741 (1.3%); Elemental C, 56.59; H, 4.43; N, 8.80; O, 20.10; S, 10.07.

### 3-Octylcarbamoyl-7-oxabicyclo[2.2.1]heptane-2-carboxylic acid (83)

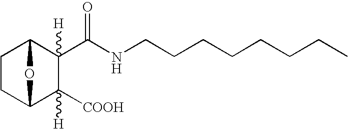

Purification afforded (83) (0.60 g, 35%) as a white crystalline solid. Mp 131° C.; ¹H NMR (300 MHz) (DMSO-d₆) δ 11.81 (bs, 1H), 7.34 (t, J=5.6 Hz, 1H), 4.71 (d, J=2.6 Hz, 1H), 4.43 (d, J=3.9 Hz, 1H), 2.94 (q, J=5.6 Hz, 2H), 2.80 (bs, 2H), 1.51-1.31 (m, 4H), 1.22 (bs, 12H), 0.84 (t, J=5.6 Hz, 3H); ¹³C NMR (75 MHz, DMSO-d₆) δ 172.3, 170.2, 78.7, 76.6, 52.9, 51.3, 38.3, 31.2, 28.9, 28.8, 28.7, 28.6, 28.3, 26.4, 22.0, 13.9; Calculated for C₁₆H₂₇NO₄; Exact Mass: 297.1940; m/e: 297.1940 (100.0%), 298.1974 (17.8%), 299.2007 (1.5%); Elemental C, 64.62; H, 9.15; N, 4.71; O, 21.52.

### 3-Decylcarbamoyl-7-oxabicyclo[2.2.1]heptane-2-carboxylic acid (85)

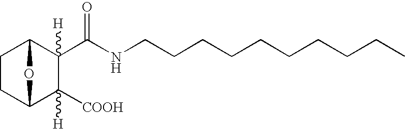

Purification afforded (85) (0.47 g, 49%) as a white crystalline solid. Mp 110-111° C.; ¹H NMR (300 MHz) (DMSO-d₆) δ 12.01 (bs, 1H), 7.33 (t, J=5.6 Hz, 1H), 4.71 (d, J==2.6 Hz, 1H), 4.43 (d, J==3.9 Hz, 1H), 2.94 (t, J=5.6 Hz, 2H), 2.80 (bs, 2H), 1.54-1.28 (m, 4H), 1.22 (bs, 16H), 0.84 (t, J=5.6 Hz, 3H); ¹³C NMR (75 MHz, DMSO-d₆) δ 172.3, 170.2, 78.7, 76.2, 52.9, 51.3, 38.4, 31.2, 28.9, 28.8, 28.7, 28.6, 28.3, 26.4, 22.0, 13.8; Calculated for C₁₈H₃₁NO₄; Exact Mass: 325.2253; m/e: 325.2253 (100.0%), 326.2287 (20.0%), 327.2320 (1.9%); Elemental C, 66.43; H, 9.60; N, 4.30; O, 19.66.

### 3-Allylcarbamoyl-7-oxabicyclo[2.2.1]heptane-2-carboxylic acid (86)

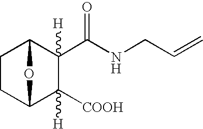

Purification afforded (86) (0.16 g, 13%) as a white crystalline solid. Mp 105-107° C.; ¹H NMR (300 MHz) (DMSO-d₆) δ 12.01 (bs, 1H), 7.49 (t, J=5.6 Hz, 1H), 5.82-5.76 (m, 1H), 5.13-5.06 (m, 2H), 4.67 (d, J=2.6 Hz, 1H), 4.49 (d, J=3.2 Hz, 1H), 3.72 (d, J=2.6 Hz, 2H), 2.82 (d, J=9.6 Hz, 1H), 2.74 (d, J=9.6 Hz, 1H), 1.52-1.46 (m, 4H); ¹³C NMR (75 MHz, DMSO-d₆) δ 173.2, 171.0, 135.4, 118.0, 114.9, 78.4, 77.2, 53.3, 52.8, 28.7, 28.5; Calculated for C₁₁H₁₅NO₄; Exact Mass: 225.1001; m/e: 225.1001 (100.0%), 226.1035 (12.2%); Elemental C, 58.66; H, 6.71; N, 6.22; O, 28.41

### 3-(4-hydroxyphenylcarbamoyl)-7-oxabicyclo[2.2.1]heptane-2-carboxylic acid (87)

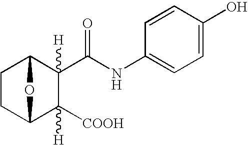

Purification afforded (87) (0.75 g, 82%) as a light purple crystalline solid. Mp 154-156° C.; ¹H NMR (300 MHz) (DMSO-d₆) δ 11.68 (bs, 1H), 9.29 (bs, 1H), 9.08 (bs, 1H), 7.27 (d, J=8.6 Hz, 2H), 6.65 (d, J=8.6 Hz, 2H), 4.76 (d, J=2.8 Hz, 1H), 4.59 (d, J=4.0 Hz, 1H), 2.99 (d, J=9.6 Hz, 1H), 2.90 (d, J=9.6 Hz, 1H), 1.15-1.47 (m, 4H); ¹³C NMR (75 MHz, DMSO-d₆) δ 172.2, 168.6, 153.1, 130.8, 120.9, 114.8, 78.6, 76.7, 53.3, 51.5, 28.9, 28.3; Calculated for C₁₄H₁₅NO₅; Exact Mass: 277.0950; m/e: 277.0950 (100.0%), 278.0984 (15.6%), 279.1017 (1.1%), 279.0993 (1.0%); Elemental C, 60.64; H, 5.45; N, 5.05; O, 28.85.

### 3-(2-Ethylphenylcarbamoyl)-7-oxa-bicyclo[2.2.1]heptane-2-carboxylic acid (88)

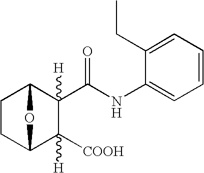

Purification afforded (88) (0.43 g, 42%) as a white crystalline solid. Mp 171-172° C.; ¹H NMR (300 MHz) (DMSO-d₆) δ 12.07 (bs, 1H), 8.72 (bs, 1H), 7.65 (d, J=8.8 Hz, 1H), 7.18-7.01 (m, 3H), 4.84 (d, J=2.8 Hz, 1H), 4.72 (d, J=3.6 Hz, 1 H), 3.10 (d, J=9.6 Hz, 1H), 3.05 (d, J=9.6 Hz, 1H), 2.55 (q, J=5.8 Hz, 2H), 1.67-1.53 (m, 4H), 1.12 (t, J=5.8 Hz, 3H); ¹³C NMR (75 MHz, DMSO-d₆) δ 172.2, 169.4, 135.7, 135.1, 128.2, 125.8, 124.4, 123.2, 79.0, 77.0, 54.1, 51.7, 28.43, 28.38, 23.5, 14.0; Calculated for C₁₆H₁₉NO₄; Exact Mass: 289.1314; m/e: 289.1314 (100.0%), 290.1348 (17.8%), 291.1381 (1.5%); Elemental C, 66.42; H, 6.62; N, 4.84; O, 22.12.

### Coumpound (89)

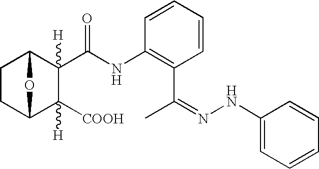

Purification afforded (89) (0.12 g, 9%) as an off-white solid. ¹H NMR (300 MHz) (DMSO-d₆) δ 12.01 (bs, 1H), 10.69 (bs, 1H), 9.22 (bs, 1H), 8.13 (d, J=8.1 Hz, 1H), 7.46 (d, J=7.8 Hz, 1H), 7.26-7.21 (m, 5H), 7.09 (t, J=7.4 Hz, 1H), 6.08-6.77 (m, 1H), 4.71 (d, J=3.1 Hz, 1H), 4.62 (d, J=2.6 Hz, 1H), 2.94 (d, J=9.7 Hz, 1H), 2.85 (d, J=9.7 Hz, 1H), 2.29 (s, 3H), 1.49-1.29 (m, 4H); ¹³C NMR (75 MHz, DMSO-d₆) δ 171.9, 169.1, 145.7, 143.1, 135.8, 128.8, 128.4, 128.2, 127.6, 123.0, 121.5, 119.2, 112.9, 78.2, 76.8, 54.3, 51.9, 30.5, 28.3, 15.9; Calculated for C₂₂H₂₃N₃O₄; Exact Mass: 393.1689; m/e: 393.1689 (100.0%), 394.1722 (24.5%), 395.1756 (2.9%), 394.1659 (1.1%); Elemental C, 67.16; H, 5.89; N, 10.68; O, 16.27

### 3-[4-(Ethoxycarbonyl)-1H-pyrazol-3-ylcarbamoyl]-7-oxabicyclo[2.2.1]heptane-2-carboxylic acid (90)

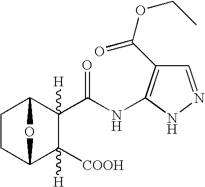

Purification afforded (90) (0.13 g, 12%) as a white crystalline solid. ¹H NMR (300 MHz) (DMSO-d₆) δ 12.07 (bs, 1H), 9.86 (bs, 1H), 7.75 (bs, 1H), 4.90 (d. J=3.6 Hz, 1H), 4.79 (d, J=4.2 Hz, 1H), 4.26-4.18 (m, 2H); 3.16 (bs, 2H), 1.69-1.56 (m, 4H), 1.28 (t, J=7.09 Hz, 3H); ¹³C NMR (75 MHz, DMSO-d₆) δ 171.8, 169.5, 162.5, 141.6, 137.6, 98.8, 78.6, 77.2, 59.5, 54.2, 52.1, 28.5, 28.0, 14.1; Calculated for C₁₄H₁₇N₃O₆; Exact Mass: 323.1117; m/e: 323.1117 (100.0%), 324.1151 (15.6%), 325.1160 (1.2%), 325.1184 (1.1%), 324.1088 (1.1%); Elemental C, 52.01; H, 5.30; N, 13.00; O, 29.69

### 3-(4-tert-Butylphenylcarbamoyl)-7-oxabicyclo[2.2.1]heptane-2-carboxylic acid (91)

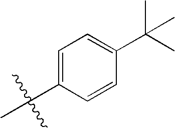

¹H NMR (DMSO-d₆): 11.89 (1H, brs), 9.48 (1H, s, 1H, NH), 7.42 (2H, d, J=8.4 Hz), 7.26 (2H, d, J=8.4 Hz), 4.77 (1H, s, 1H, CH), 4.61 (1H, d, J=3.3 Hz), 3.03 (1H, d, J=9.6 Hz), 2.91 (1H, d, J=9.6 Hz), 1.45-1.60 (4H, m), 1.24 (9H, s). ¹³C NMR (DMSO-d₆): 173.1, 169.9, 146.1, 137.5, 126.0 (2C), 119.8, 119.7, 54.4, 52.5, 34.8, 32.0 (3C), 29.8, 29.3. MP: 179° C. HRMS (M+H) calcd 318.1705, found 318.1699.

### 3-(2,4-Di-tert-Butylphenylcarbamoyl)-7-oxabicyclo[2.1.1]heptane-2-carboxylic acid (92)

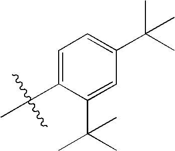

¹H NMR (DMSO-d₆): 1.25 (18H, s), 1.49-1.62 (4H, m), 2.89 (2H, d, J=9.5 Hz), 3.04 (2H, d, J=9.5 Hz), 4.61 (1H, d, J=4.0 Hz, 1H, CH), 4.84 (1H, s, 1H, CH), 7.05 (1H, s, 1H, CH—Ar), 7.39 (2H, s, 2H, 2CH—Ar), 9.50 (1H, s, 1H, NH), 10.9 (1H, brs, 1H, COOH). ¹³C NMR (DMSO-d₆): 173.2, 169.9, 151.4, 139.6, 117.5, 114.3, 79.8, 77.8, 54.6, 52.3, 35.3, 32.1 (6C), 29.8, 29.4. MP: 182° C.

### 3-(2-Dimethylaminoethylcarbamoyl)-7-oxabicyclo[2.2.1]heptane-2-carboxylic acid (93)

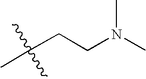

¹H NMR: (CDCl₃): 7.34 (1H, t, J=5.5 Hz), 5.00 (1H, d, J=4.65 Hz), 4.76 (1H, d, J=2.1 Hz) 4.67 (1H, d, J=4.8 Hz), 3.57 (2H, q, J=6.3 Hz) 3.07 (1H, d, J=9.6 Hz), 2.96 (1H, d, J=9.6 Hz), 2.43 (2H, t, J=6.8 Hz), 2.22 (6H, s) 1.58-1.50 (4H, m). ¹³C NMR: (CDCl₃): 172.3, 170.3, 78.7, 76.7, 53.0, 51.4, 49.4, 44.8, 36.3, 28.8, 28.3. MP: 45-47° C. HRMS (M+H) calcd 257.1501, found 257.1509.

### 3-(3-Dimethylaminopropylcarbamoyl)-7-oxabicyclo[2.2.1]heptane-2-carboxylic acid (94)

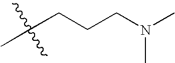

¹H NMR: (CDCl3): 7.34 (1H, t, J=5.5 Hz), 5.02 (1H, d, J=4.5 Hz), 4.80 (1H, d, J=2.0 Hz) 4.70 (1H, d, J=4.2 Hz), 3.57 (2H, q, J=7.4 Hz), 3.10 (1H, d, J=9.6 Hz), 2.96 (1H, d, J=9.6 Hz), 2.60 (2H, t, J=6.7 Hz), 2.22 (6H, s), 1.66 (2H, quin, J=7.4 Hz), 1.58-1.50 (4H, m). ¹³C NMR: (CDCl₃): 175.5, 174.3, 78.5, 76.3, 53.0, 51.2, 49.2. 44.7, 36.7, 28.5, 28.1, 24.9. MP: 42-44° C. HRMS (M+H) calcd 271.1658, found 271.1666.

### 3-(2-Morpholin-4-ylethylcarbamoyl)-7-oxabicyclo[2.2.1]heptane-2-carboxylic acid (95)

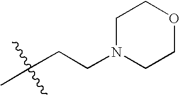

¹H NMR: (DMSO-d6): 4.65 (1H, m), 3.77 (2H, m), 3.51 (2H, m), 3.16 (1H, d, J=9.6 Hz), 3.00 (1H, d, J=9.5 Hz), 2.51 (2H, m), 1.52 (2H, m). ¹³C NMR: (DMSO-d6): 176.1, 173.5, 82.0, 81.5, 56.7, 52.3, 51.8, 47.8, 32.8, 32.2, 30.5, 30.3. MP: 116-119° C. HRMS: C₁₄H₂₂N₂O₅ requires 299.1607; Found 299.1610.

### 3-(3,4-Difluorophenylcarbamoyl)-7-oxabicyclo[2.2.1]heptane-2-carboxylic acid (96)

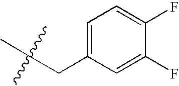

¹H NMR: (DMSO-d₆): 9.88 (1H, s, NH), 7.69 (1H, m), 7.30 (1H, m), 7.19 (1H, m), 4.76 (1H, br), 4.63 (1H, br), 2.97 (2H, m), 1.67-1.47 (4H, m). ¹³C NMR: (DMSO-d₆): 172.0, 169.6, 150.5, 147.3, 143.5, 136.1, 117.2, 108.0, 78.4, 76.9, 53.2, 51.8, 28.8, 28.3. MP: 174-176° C. HRMS (M+H) calcd 298.0891, found 298.0893.

### 3-(4-Trifluoromethylphenylcarbamoyl)-7-oxabicyclo[2.2.1]heptane-2-carboxylic acid (97)

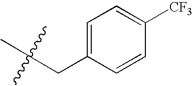

¹H NMR: (DMSO-d₆): 11.97 (1H, brs), 10.01 (1H, s, NH), 7.73 (2H, d, J=8.4 Hz), 7.61 (2H, d, J=8.4 Hz), 4.77 (1H, br), 4.66 (1H, br), 3.07 (1H, d, J=9.6 Hz), 2.96 (1H, d, J=9.6 Hz), 1.58-1.50 (4H, m). ¹³C NMR: (DMSO-d₆): 172.0, 169.8, 142.7, 126.1-123.5 (d, J=894.0 Hz), 125.8 (q, J=15.0 Hz), 123.5, 123.1, 122.7, 122.5, 122.3 (sextet, J=60 Hz), 78.4, 76.9, 53.4, 51.8, 28.8, 28.3. MP: 189-191° C. HRMS (M+H) calcd 330.0953, found 330.0955.

### Compound (103)

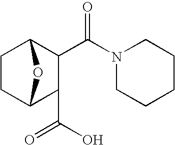

¹H NMR (DMSO): 4.70 (2H, q J=2.4 Hz), 4.63 (2H, m), 4.60 (2H, m), 2.75 (2H, s), 1.62 (2H, m), 1.60-1.49 (8H, m). ¹³C NMR (DMSO): 173.84, 172.7, 77.84, 77.81, 53.58, 53.50, 43.7, 29.0, 28.6, 28.4, 22.3. MP 140-142° C.

### Compound (105)

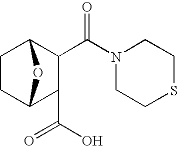

¹H NMR (DMSO-d₆): 1.47-151 (m, 4H, 2×CH₂), 2.49-2.51 (m, 2H, CH₂), 2.52-2.60 (m, 2H, CH₂), 2.99 (d, J=, 9.6 Hz, 1H, CH), 3.14 (d, J=9.6 Hz, 1H, CH), 3.47-3.56 (m, 2H, CH₂), 3.73-3.79 (m, 2H, CH₂), 4.62-4.65 (m, 2H, CH₂). ¹³C NMR (DMSO-d₆): 27.3 (CH₂), 27.4 (CH₂), 29.1 (CH₂), 29.8 (CH₂), 44.7 (CH₂), 48.8 (CH₂), 49.2 (CH), 53.7 (CH), 78.4 (CH), 79.0 (CH), 170.4 (CON), 173.0 (COOH). MP 210° C. dec

### Compound (109)

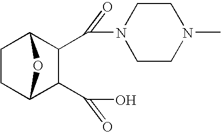

¹H NMR (DMSO): 4.63 (2H, d), 3.84 (2H, m, br), 3.12 (1H, d J=9.5 Hz), 2.98 (1H, d J=9.5 Hz), 2.34 (1H, m br), 2.16 (4H, s), 1.51-1.46 (4H, m). ¹³C NMR (DMSO): 172.1, 169.3, 77.8, 77.6, 53.9, 53.7, 52.7, 48.4, 45.4, 28.7, 28.3. MP 228-230° C.

### Compound (128)

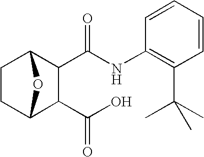

¹H NMR (DMSO-d₆): 1.32 (s, 9H), 1.54-1.60 (m, 4H, 2×CH₂), 3.08 (dd, J=13.6 and 10.0 Hz, 2H, CH₂), 4.69 (d, J=4.5 Hz, 1H, CH), 4.84 (s, 1H, CH), 7.12-7.15 (m, 2H, 2×CH—Ar), 7.28-7.35 (m, 2H, 2×CH—Ar), 8.53 (s, 1H, NH). ¹³C NMR (DMSO-d₆): 29.3 (CH₂), 29.5 (CH₂), 31.3 (3×CH₃), 35.2 (C), 52.6 (CH), 54.7 (CH), 77.9 (CH), 79.3 (CH), 126.6 (CH—Ar), 126.9 (CH—Ar), 127.0 (CH—Ar), 130.0 (CH—Ar), 136.7 (CH—Ar) 144.7 (C—Ar), 170.5 (CON), 173.2 (COOH). MP 163-164° C. HRMS Calculated 318.1661, Found 318.1706

### Example 4

### Testing of Compounds on T. vitrinus L3 Larvae

The ability of the compounds to kill T. vitrinus L3 larvae was tested. The L3 stage is encased in a sheath, which protects the larva from external stresses such as desiccation and chemicals. This makes it more difficult to kill this part of the lifecycle with chemotherapeutic agents. The tests will determine which compounds can kill this lifestage, therefore defining which compounds can penetrate through the sheath and subsequently kill the larva.

The L3 stage of T. vitrinus were collected and stored in water at 4° C. until needed. Each compound was suspended in 100% dimethyl sulphoxide (DMSO) to a final concentration of 100 mM.

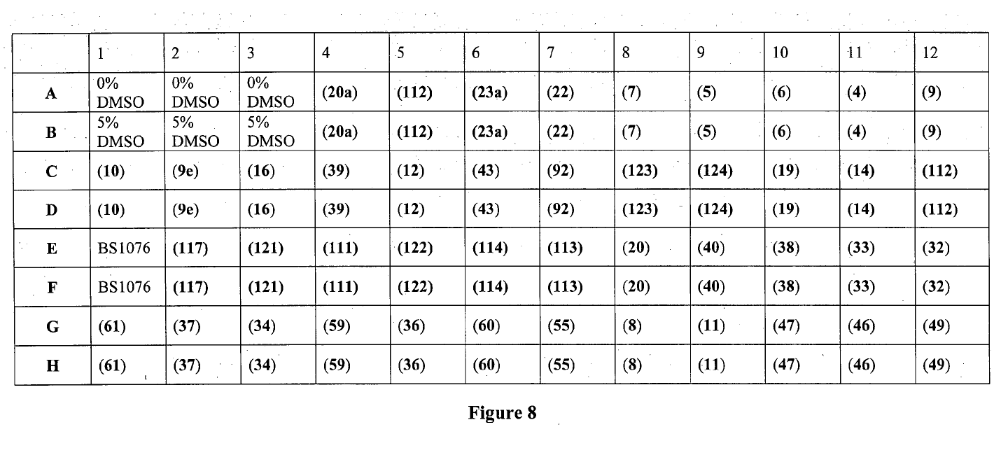

Around fifty L3 larvae were added, in 95 μl of water, to the wells of a 96-well microtitre plate. All compounds were tested initially at a concentration of 5 mM by adding 5 μl of compound in 100% DMSO, as shown in FIG. 8. The plate was incubated overnight RT. Plates were viewed under a binocular microscope and the proportion of dead L3 larvae in the controls and each treatment were scored. Concentrations above 5 mM were not tested as DMSO alone kills larvae when the proportion exceeds 5% of the total volume. Many of the compounds are predicted to have limited solubility in aqueous solution, thus requiring the presence of organic solvent. The appropriate controls included wells with and without various concentrations of DMSO alone.

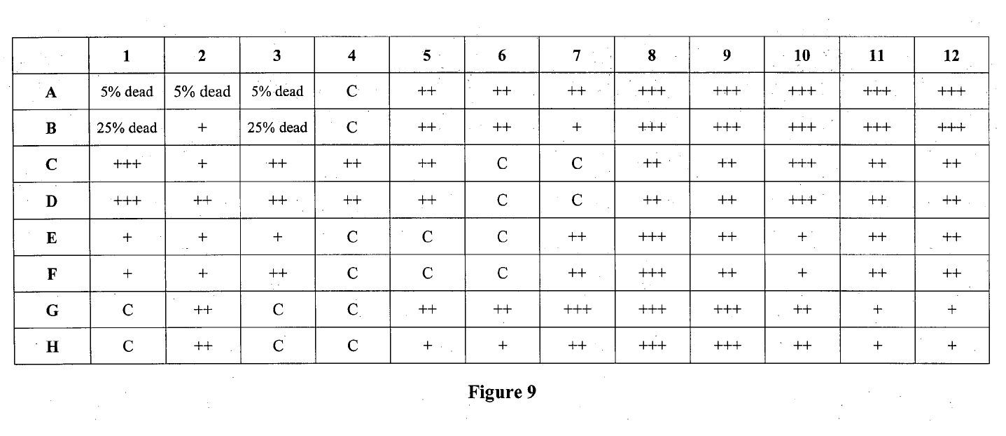

These results indicate that the initial screening plate successfully identified compounds that can kill T. vitrinus L3 larvae and defined the range of lethal concentrations of each (FIG. 9). Those compounds that kill must be able to penetrate the sheath of the larva and at a high enough concentration to be lethal. The main problem with assessing these compounds for mortality is that up to 50% mortality occurs with 5% DMSO alone. This suggests that only those compounds that show 100% mortality should be considered for secondary screening.

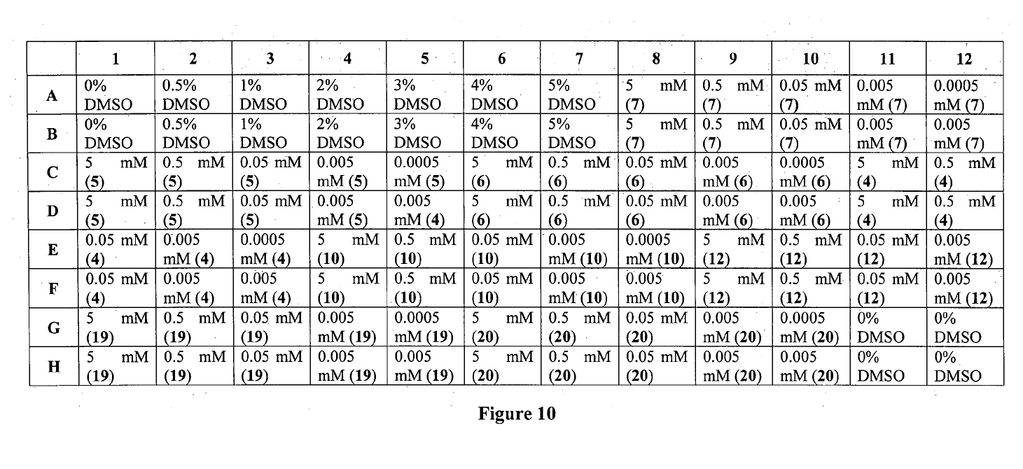

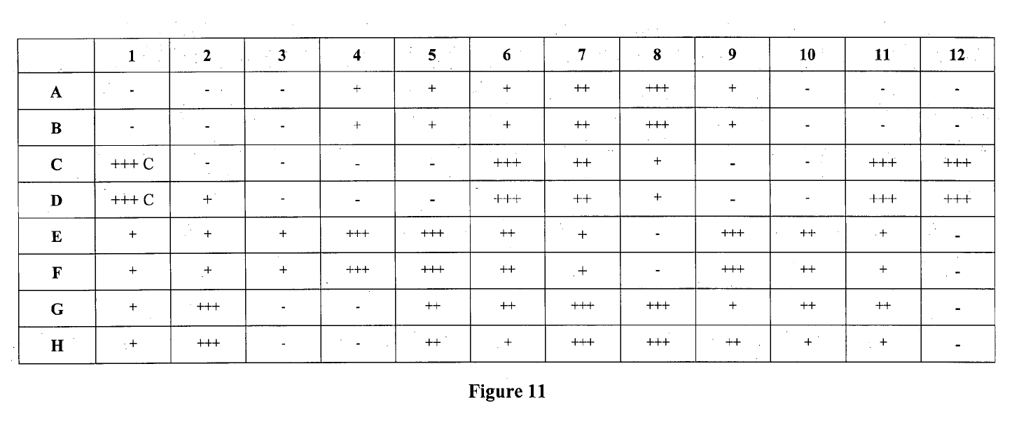

Of the compounds tested, eight compounds showed an ability to kill 100% of the T. vitrinus L3 larvae added ((7), (5), (6), (4), (10), (12), (19) and (20)) and were selected for secondary screening. As shown in FIG. 10, the compounds were tested in serial ten-fold dilutions down to 0.5 μM, confirmed that these compounds could kill all larvae exposed at 5 mM. Three compounds ((4), (10) and (20)) could also kill 100% of larvae exposed at 0.5 mM. None of the compounds showed significant mortality at lower concentrations. This series of experiments clearly demonstrates that the tested compounds can kill T. vitrinus L3 larvae, the most difficult stage of the lifecycle to kill.

As the skilled addressee will appreciate, the inhibitors of the invention may act upon phosphatases other than Tv-stp1 described herein. Furthermore, even though compounds related to those described herein may act upon phosphatases it is possible that they are acting through other invertebrate proteins/molecules.

Further tests can be performed on the other life cycle stages of larvae to determine their ability to kill.

### Example 5

### Testing Compounds on H. contortus L3 Larvae

The L3 stage of H. contortus was collected from the faeces of experimentally infected sheep and stored in water at room temperature until needed. A stock solution of each compound was prepared by suspending the compound in 100% dimethyl sulphoxide (DMSO) to a final concentration of 100 mM.

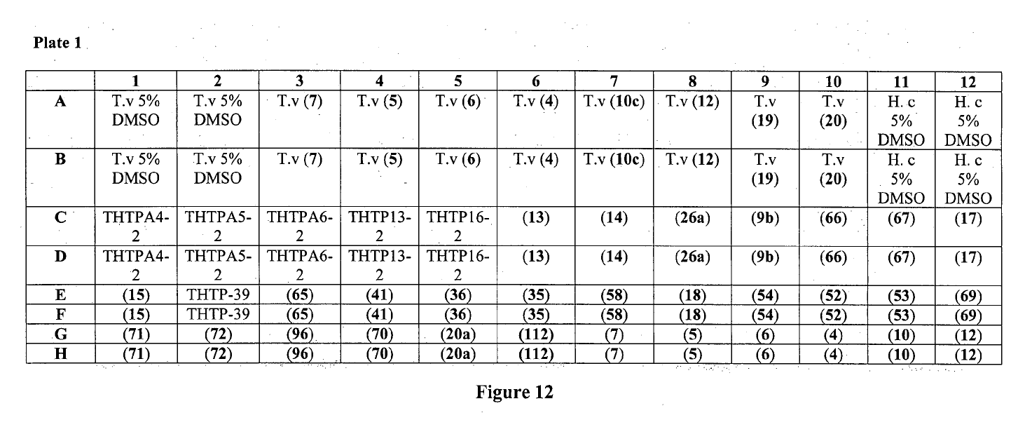

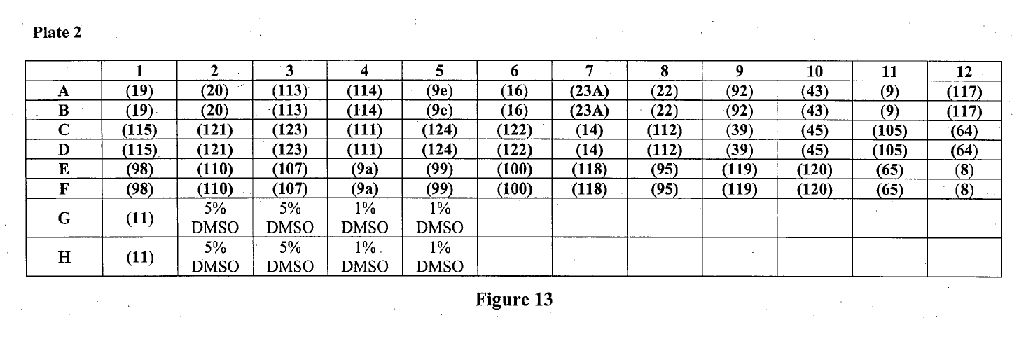

For this set of experiments, Trichostrongylus vitrinus (T. vitrinus) L3 larvae were used as positive controls by testing with the compounds identified in previous experiments as being able to kill all larvae. Approximately 50 L3 larvae of each species were added, in 95 μl of water, to the wells of a 96-well microtitre plate. All compounds were tested initially at a concentration of 5 mM by adding 5 μl of inhibitor in 100% DMSO. The loading of the 96 well microtitre plate is shown in FIGS. 12 and 13.

The plates were incubated overnight at room temperature (RT). Plates were viewed under a binocular microscope and the compounds showing 100% kill of H. contortus larvae were determined. Concentrations above 5 mM were not tested as DMSO alone kills larvae when the proportion exceeds 5% of the total volume. Many of the compounds are predicted to have limited solubility in aqueous solution, thus requiring the presence of organic solvent. Additional controls included wells with and without various concentrations of DMSO alone.

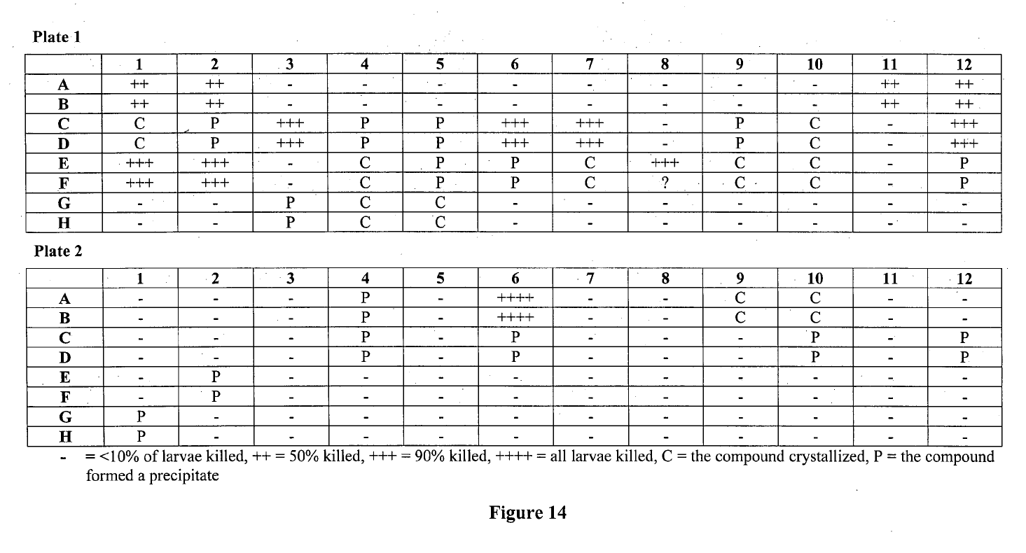

As shown in FIG. 14, eight compounds killed 90-100% of the H. contortus L3 larvae. Of these, only one appeared to kill all larvae in the wells. The compounds that killed H. contortus L3 larvae were: (13), (14), (17), (15), THTP-39, (18) and (16).

These compounds are different from those that were found to kill T. vitrinus. Those compounds found to kill H. contortus L3 larvae were titrated to determine how toxic each chemical structure is.

### Secondary Screening

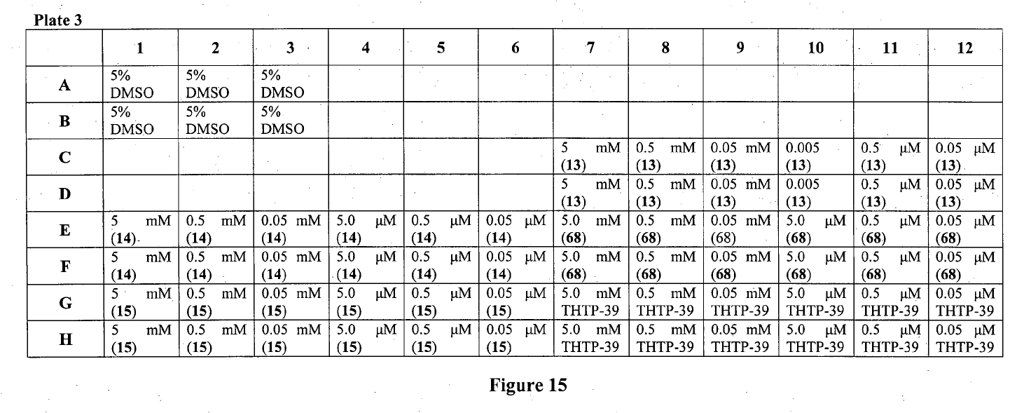

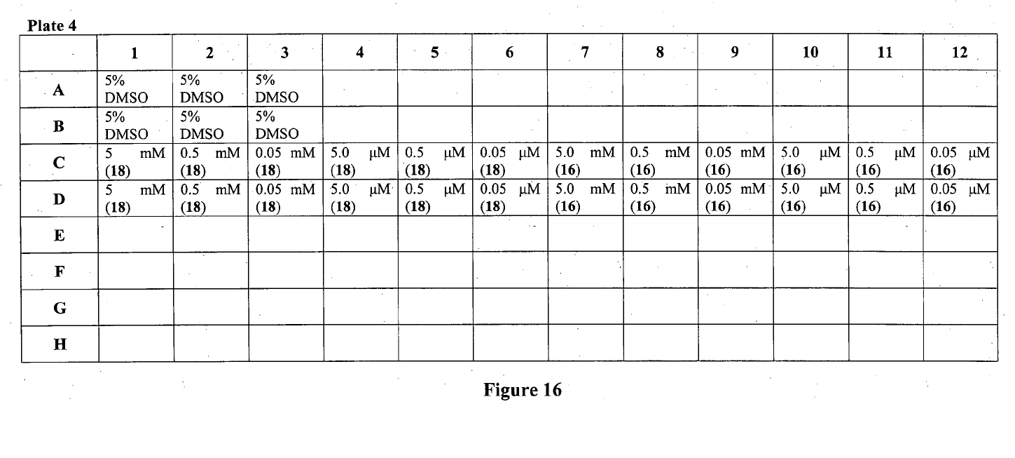

Eight inhibitors, from the primary screening, appeared to kill 90-100% of H. contortus L3 larvae at a concentration of 5 mM. Sequential ten-fold dilutions of 5 mM, down to 0.5 were screened for the ability to kill all larvae added to the well, as shown in FIGS. 15 and 16 for plates 3 and 4. This allowed a direct comparison of toxicity between compounds.

### Results

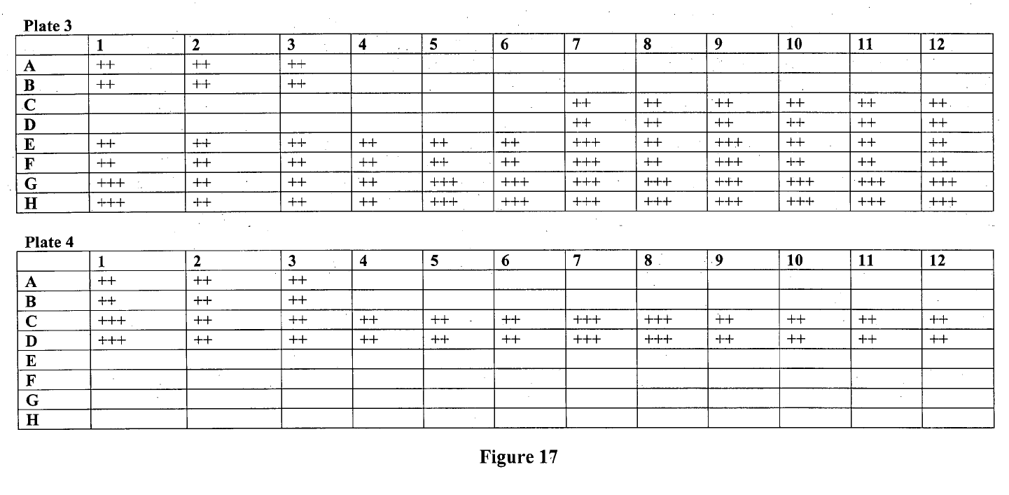

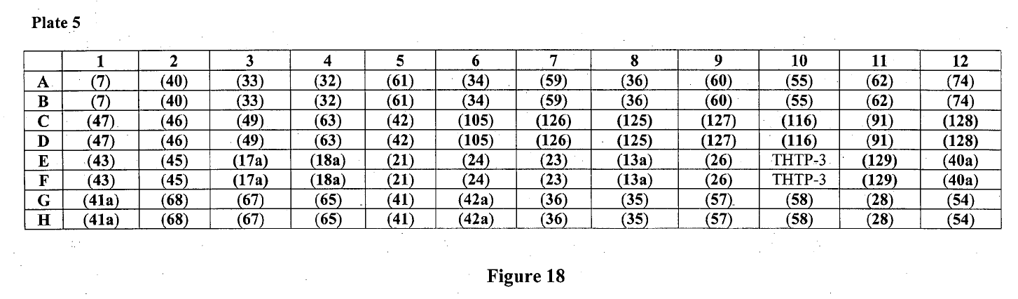

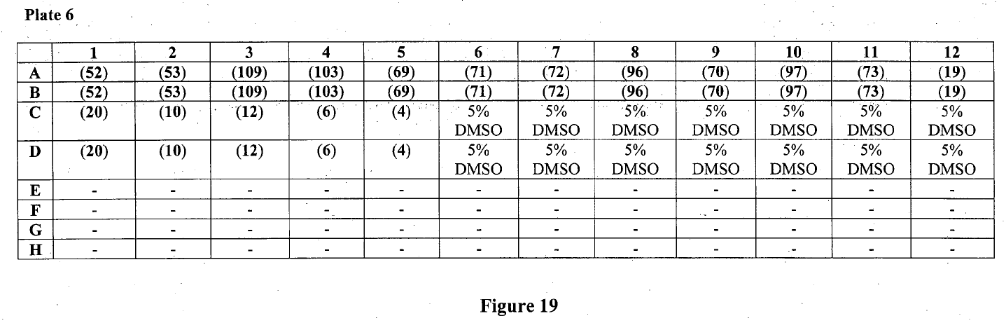

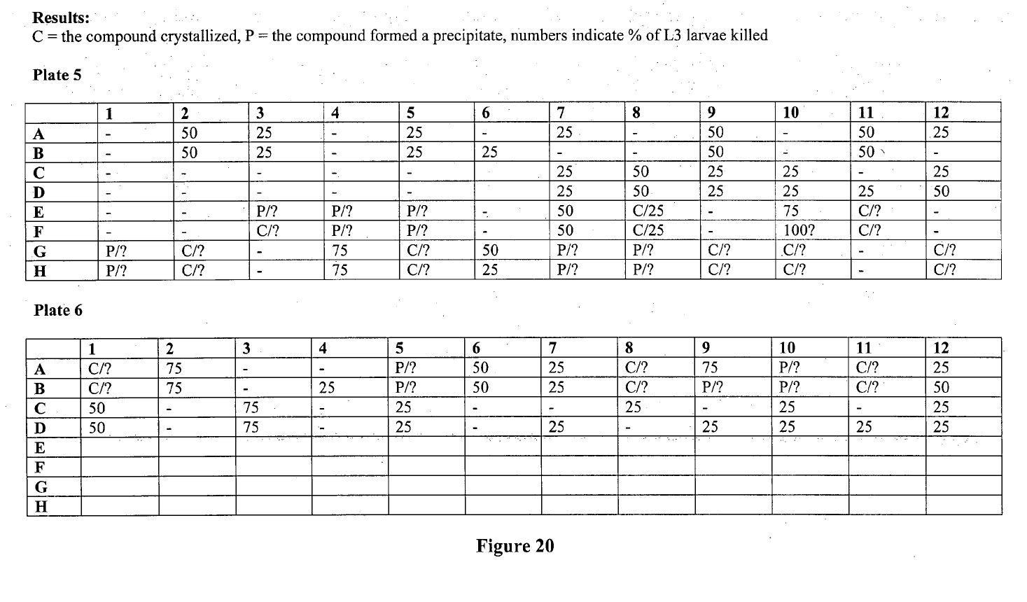

Of the eight inhibitors tested a second time, this time using a dilution series, five appeared to kill 90% of the L3 larvae in the assay, as shown in FIG. 17. Some compounds, such as (15) killed the larvae at the highest and lowest concentrations used. This is probably due to some precipitation at the mid-range concentrations, but good aqueous solubility at the very lowest concentrations makes enough available to be toxic to the larvae.

### Example 6

### Testing of Inhibitors on Trichostrongylus colubriformis L3 Larvae

To test compounds of the invention for their ability to kill T. colubriformis, L3 larvae were used. The L3 stage is encased in a sheath, which protects the larva from external stresses such as desiccation and chemicals. This makes it more difficult to kill this part of the lifecycle with chemotherapeutic agents. The planned tests will determine which compounds can kill this lifestage, therefore defining which compounds can penetrate through the sheath and subsequently kill the larva of this species.

### Materials and Methods:

Larvae: The L3 stage of T. colubriformis was provided by Gareth Hutchinson (Elizabeth Murdoch Agricultural Institute, NSW Dept of Primary Industries, Woodbridge Road, Menangle, NSW 2568)

Chemicals: The compounds to be tested were supplied as dried powders and were resuspended in 100% dimethyl sulphoxide (DMSO) to a final concentration of 100 mM.

Microplate Assay: Approximately 50 L3 larvae of each species were added, in 95 μl of water, to the wells of a 96-well microtitre plate. All compounds were tested initially at a concentration of 5 mM by adding 5 μl of inhibitor in 100% DMSO. The plates were incubated overnight at RT. Plates were viewed under a binocular microscope and the compounds showing 100% kill of T. colubriformis larvae were determined. Concentrations above 5 mM were not tested as DMSO alone kills larvae when the proportion exceeds 5% of the total volume. Many of the compounds are predicted to have limited solubility in aqueous solution, thus requiring the presence of organic solvent.

None of the 65 compounds tested killed all of the larvae exposed, although one compound killed more than 75% of larvae. Five of the compounds killed 75% of the larvae, these compounds, together with those identified in the previous experiment will be re-tested using a dilution series of each compound. It is possible that some of the compounds failed to kill larvae due to poor solubility in aqueous solution (P and C).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida and Allshire (2005) TRENDS Cell Biol 15: 251-258.
Armstrong et al. (1995) J Cell Sci. 108: 3367-3375.
Barford et al. (1998) Ann. Rev. Biophys. Biomol. Struct. 27: 133-164.
Boag et al. (2003) Int J Parasitol. 2003 33:313-325.
Dunbrack et al. (1997). Folding and Design, 2:R27-42.
Egloff et al. (1997) EMBO J. 16, 1876-1887.
Gasser et al. (1993) Nucleic Acids Res. 21: 2525-2526.
Gupta et al. (1997) J. Med. Chem. 40:3199-3206.
Hart et al. (2004) Bioorganic & Medicinal Chemistry Letters 14:1969-1973.
Harayama (1998). Trends Biotechnol 16: 76-82.
Haseloff and Gerlach (1988) Nature 334:585-591.
Herrmann et al. (1998) Chromosoma 107: 440-451.
Hoste et al. (1995) Int. J. Parasitol. 25: 75-80.
Hu et al. (2005) Parasitology 130: 117-129.
Huang (1992) Genomics 14: 18-25.
MAFF (1977) Manual of Veterinary Parasitological Laboratory Techniques.
Technical Bulletin 18. Her Majesty's Stationary Office, London, UK.
Millar and Waterhouse (2005) Funct Integr Genomics 5:129-135.
Nakamura et al. (1999) Biochem. J. 344: 819-825.
Needleman and Wunsch (1970) J. Mol. Biol. 48:443-453.
Nisbet and Gasser (2004) Int. J. Parasitol. 34: 633-643.
Pasquinelli et al. (2005) Curr Opin Genet Develop 15: 200-205.
Perriman et al. (1992) Gene 113: 157-163.
Shippy et al. (1999) Mol. Biotech. 12: 117-129.
Smith et al. (1996) Biol. Reprod. 54: 719-727.
Smith et al. (2000) Nature 407: 319-320.
Thompson et al. (1994) Nucleic Acids Res. 22: 4673-4680.
Waterhouse et al. (1998) Proc. Natl. Acad. Sci. USA 95: 13959-13964.

1. A method of controlling an invertebrate pest, comprising contacting said pest with at least one invertebrate control agent(s) having the Formula (IIIA): 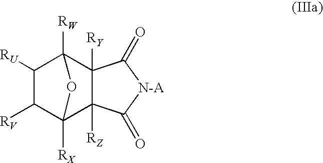
wherein:
A is independently selected from H, a substituted or unsubstituted aliphatic, alicyclic or aryl group, or a substituted or unsubstituted aliphatic, alicyclic or aryl group in which the carbon chain is interrupted by one or more heteroatom(s);
each of R_{U} and R_{V} is independently selected from H, NR⁵R⁶, OR⁷, a substituted or unsubstituted aliphatic, alicyclic or aryl group, or a substituted or unsubstituted aliphatic, alicyclic or aryl group in which the carbon chain is interrupted by one or more heteroatom(s), or together form an epoxide ring,
each of R⁵, R⁶, R⁷, R_{W}, R_{X}, R_{Y} and R_{Z} is independently selected from H, a substituted or unsubstituted aliphatic, or a substituted or unsubstituted aliphatic group in which the carbon chain is interrupted by one or more heteroatom(s);
and a salt thereof, and a solvate thereof.

2. The method of claim 1, wherein each of R_{U}, R_{V}, R_{W}, R_{X}, R_{Y} and R_{Z} is H.

3. (canceled)

4. (canceled)

5. (canceled)

6. (canceled)

7. The method of claim 1, wherein A is a C₃₋₁₀ alkyl group.

8. The method of claim 1, wherein A is an alkyl group selected from the group consisting of —CH₂CH₂CH₃, —CH₂(CH₂)₂CH₃, —CH₂(CH₂)₆CH₃ and —CH₂(CH₂)₈CH₃.

9. (canceled)

10. The method of claim 1, wherein A is an alkyl group substituted with an alicyclic group selected from the group consisting of

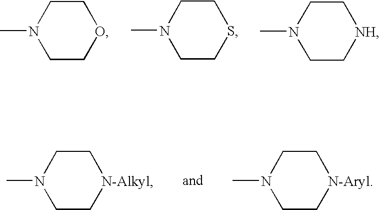

11. (canceled)

12. The method of claim 1, wherein A is a C₃₋₁₀ alkene.

13. (canceled)

14. (canceled)

15. The method of claim 1, wherein A is an alkene is selected from the group consisting of —CH₂CH═CH₂; —CH₂CH₂CH═CH₂, —CH₂(CH₂)₃CH═CH₂ and —CH₂(CH₂)₅CH═CH₂.

16. (canceled)

17. (canceled)

18. (canceled)

19. (canceled)

20. (canceled)

21. (canceled)

22. (canceled)

23. (canceled)

24. (canceled)

25. (canceled)

26. The method of claim 1, wherein the invertebrate pest is a helminth selected from the group consisting of nematodes, cestodes and trematodes.

27. (canceled)

28. The method of claim 1, wherein the invertebrate pest is a nematode is at least one of the group consisting of Trichostrongylus sp, Haemonchus sp., Ostertagia, Cooperia, Oesphagostomum, Nematodirus and Dictyocaulus.

29. (canceled)

30. (canceled)

31. (canceled)

32. (canceled)

33. The method of claim 1, wherein the invertebrate control agent is at least one of the group selected from the group consisting of:
N-propyl-7-Oxabicyclo(2.2.1)heptane-2,3-dicarboximide;
N-butyl-7-Oxabicyclo(2.2.1)heptane-2,3-dicarboximide;
N-octyl-7-Oxabicyclo(2.2.1)heptane-2,3-dicarboximide;
N-decyl-7-Oxabicyclo(2.2.1)heptane-2,3-dicarboximide;
N-(3-butenyl)-7-Oxabicyclo(2.2.1)heptane-2,3-dicarboximide;
N-Butanoic acid-7-Oxabicyclo(2.2.1)heptane-2,3-dicarboximide;
N-Hexanoic acid-7-Oxabicyclo(2.2.1)heptane-2,3-dicarboximide;
N-Ethyl alcohol-7-Oxabicyclo(2.2.1)heptane-2,3-dicarboximide;
N-2-Methoxypropyl-1-ol-7-Oxabicyclo(2.2.1)heptane-2,3-dicarboximide;
4-Ethyl-7-Oxabicyclo(2.2.1)heptane-2,3-dicarboximide;
4-Sec-butyl-7-Oxabicyclo(2.2.1)heptane-2,3-dicarboximide;
4-Hexyl-7-Oxabicyclo(2.2.1)heptane-2,3-dicarboximide;
4-Cyclohexyl-7-Oxabicyclo(2.2.1)heptane-2,3-dicarboximide;
4-Dodecyl-7-Oxabicyclo(2.2.1)heptane-2,3-dicarboximide;
4-Tetradecyl-7-Oxabicyclo(2.2.1)heptane-2,3-dicarboximide;
4-Octadecyl-7-Oxabicyclo(2.2.1)heptane-2,3-dicarboximide;
4-Allyl-7-Oxabicyclo(2.2.1)heptane-2,3-dicarboximide;
4-Oxiranylmethyl-7-Oxabicyclo(2.2.1)heptane-2,3-dicarboximide;
4-(3-Hydroxypropyl)-7-Oxabicyclo(2.2.1)heptane-2,3-dicarboximide;
4-(6-Hydroxyhexyl)-7-Oxabicyclo(2.2.1)heptane-2,3-dicarboximide;
4-(2-Hydroxy-1-methylethyl)-7-Oxabicyclo(2.2.1)heptane-2,3-dicarboximide;
4-(2-Hydroxy-1,1-dimethylethyl)-7-Oxabicyclo(2.2.1)heptane-2,3-dicarboximide;
4-(1-Hydroxymethylpropyl)-7-Oxabicyclo(2.2.1)heptane-2,3-dicarboximide;
4-(2,3-Dihydroxypropyl)-10-oxa-4-azatricyclo[5.2.1]decane-3,5-dione;
4-(3-Hydroxy-2-methoxypropyl)-10-oxa-4-azatricyclo[5.2.1]decane-3,5-dione;
4-Morpholin-4-yl-7-Oxabicyclo(2.2.1)heptane-2,3-dicarboximide;
4-(2-Morpholin-4-ylethyl)-7-Oxabicyclo(2.2.1)heptane-2,3-dicarboximide dione;
4-(3-Morpholin-4-ylpropyl)-7-Oxabicyclo(2.2.1)heptane-2,3-dicarboximide;
4-Phenyl-7-Oxabicyclo(2.2.1)heptane-2,3-dicarboximide;
4-(4-Hydroxyphenyl)-10-oxa-4-azatricyclo[5.2.1]decane-3,5-dione;
4-(4-Nitrophenyl)-7-Oxabicyclo(2.2.1)heptane-2,3-dicarboximide;
4-(3,5-Dioxo-10-oxa-4-azatricyclo[5.2.1]dec-4-yl)benzoic acid;
4-Benzyl-10-oxa-4-azatricyclo[5.2.1]decane-3,5-dione;
4-(4-Methoxybenzyl)-7-Oxabicyclo(2.2.1)heptane-2,3-dicarboximide;
4-(3,4-Dimethoxybenzyl)-7-Oxabicyclo(2.2.1)heptane-2,3-dicarboximide;
4-(2-Aminobenzyl)-7-Oxabicyclo(2.2.1)heptane-2,3-dicarboximide;
Bis-3,6-epoxycyclohexane-1,2-dicarboximido)-trimethylene (Bis-norcantharimide-propyl linker);
Bis-3,6-epoxycyclohexane-1,2-dicarboximido)-dodecylmethyle (Bis-norcantharimide-dodecyl linker);
a salt thereof, and a solvate thereof.

34. (canceled)

35. A compound of formula (IIIa): 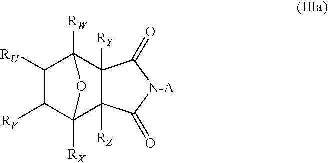
wherein:
A is independently selected from H, a substituted or unsubstituted aliphatic, alicyclic or aryl group, or a substituted or unsubstituted aliphatic, alicyclic or aryl group, in which the carbon chain is interrupted by one or more heteroatom(s);
each of R_{U} and R_{V} is independently selected from H, NR⁵R⁶, OR⁷, a substituted or unsubstituted aliphatic, alicyclic or aryl group, or a substituted or unsubstituted aliphatic, alicyclic or aryl group, in which the carbon chain is interrupted by one or more heteroatom(s), or together form an epoxide ring,
each of R⁵, R⁶, R⁷, R_{W}, R_{X}, R_{Y} and R_{Z} is independently selected from H, a substituted or unsubstituted aliphatic group, or a substituted or unsubstituted aliphatic group in which the carbon chain is interrupted by one or more heteroatom(s);
and a salt thereof, and a solvate thereof;
with the proviso that the compound is not selected from the group consisting of:
3-Propylcarbamoyl-7-oxabicyclo[2.2.1]heptane-2-carboxylic acid;
3-ethylcarbamoyl-7-oxabicyclo[2.2.1]heptane-2-carboxylic acid;
3-methylcarbamoyl-7-oxabicyclo[2.2.1]heptane-2-carboxylic acid;
N-(2-bromoethyl)-7-Oxabicyclo(2.2.1)heptane-2,3-dicarboximide;
N-(2-hydroxypropyl)-exo-(Z)-7-Oxabicyclo(2.2.1)heptane-2,3-dicarboximide;
N-phenyl-exo-(Z)-7-Oxabicyclo(2.2.1)heptane-2,3-dicarboximide;
N-exo-(Z)-7-Oxabicyclo(2.2.1)heptane-2,3-dicarboximide;
N-(o-methylphenyl)-exo-(Z)-7-Oxabicyclo(2.2.1)heptane-2,3-dicarboximide;
N-(p-chlorophenyl)-exo-(Z)-7-Oxabicyclo(2.2.1)heptane-2,3-dicarboximide;
N-(p-fluorophenyl)-exo-(Z)-7-Oxabicyclo(2.2.1)heptane-2,3-dicarboximide;
N-(o-chlorobenzyl)-exo-(Z)-7-Oxabicyclo(2.2.1)heptane-2,3-dicarboximide;
exo-7-oxabicyclo(2.2.1)heptane-2,3-dicarboximide;
N-(o-chlorobenzyl)-exo-(Z)-7-Oxabicyclo(2.2.1)heptane-2,3-dicarboximide;
N-(o-methoxyphenyl)-exo-(Z)-7-Oxabicyclo(2.2.1)heptane-2,3-dicarboximide;
N-(m-fluorophenyl)-exo-(Z)-7-Oxabicyclo(2.2.1)heptane-2,3-dicarboximide;
N-(m-ethoxyphenyl)-exo-(Z)-7-Oxabicyclo(2.2.1)heptane-2,3-dicarboximide;
N-(m-chloro-o-methylphenyl)-exo-(Z)-7-Oxabicyclo(2.2.1)heptane-2,3-dicarboximide;
N-(hydroxymethyl-acetate)-7-Oxabicyclo(2.2.1)heptane-2,3-dicarboximide;
N-(o-fluorophenyl)-exo-(Z)-7-Oxabicyclo(2.2.1)heptane-2,3-dicarboximide;
N-(2,4-dichlorophenyl)-exo-(Z)-7-Oxabicyclo(2.2.1)heptane-2,3-dicarboximide;
2,3-dimethyl-7-Oxabicyclo(2.2.1)heptane-2,3-dicarboxylic anhydride (cantharidin);
(3a-α,4-β,7-β,7a-α)-Hexahydro-3a-methyl-4,7-epoxyisobenzofuran-1,3-dione;
2-methyl-7-Oxabicyclo(2.2.1)heptane-2,3-dicarboxylic anhydride (palasonin);
hexahydro-3a,7a-dimethyl-(3a-α,4-β,7-β,7a-α)-4,7-Epoxybenzo(c)thiophene-1,3-dione;
hexahydro-3a-methyl-,(3a-α,4-β,7-β,7a-α)-4,7-Epoxybenzo(c)thiophene-1,3-dione;
N-[2-hydroxy-3-{4-(2-pyridyl)piperazin-1-yl}propyl]endo-7-oxabicyclo[2,2,1]heptane-2,3-dicarboximide;
N-[2-n-caproyloxy-3-{4-(2-pyridyl)piperazin-1-yl}propyl]endo-7-oxabicyclo[2,2,1]heptane-2,3-dicarboximide;
N-[2-cyclohexylcarbonyloxy-3-{4-(2-pyridyl)piperazin-1-yl}propyl]endo-7-oxa bicyclo[2,2,1]heptane-2,3-dicarboximide;
N-[2-n-hexadecanoyloxy-3-{4-(2-pyridyl)piperazin-1-yl}propyl]endo-7-oxabicyclo[2,2,1]heptane-2,3-dicarboxylmide; and
N-[2-hexadecanoyloxy-3-{4-(3-trifluoromethylphenyl)piperazin-1-yl}propyl]endo-7-oxabicyclo[2,2,1]heptane-2,3-dicarboximide.

36. The compound of claim 35, wherein each of Rᵤ, Rᵥ, R_{w}, Rₓ, R_{y} and R_{z} H.

37. (canceled)

38. (canceled)

39. The compound of claim 35, wherein A is a substituted or unsubstituted alkyl group.

40. The compound of claim 35, wherein A is a C₁₋₂₀ alkyl group.

41. The compound of claim 35, wherein A is a C₃₋₁₀ alkyl group.

42. The compound according to of claim 35, wherein A is selected from the group consisting of —CH₂CH₂CH₃, —CH₂(CH₂)₂CH₃, —CH₂(CH₂)₄—CH₃, —CH₂(CH₂)₆CH₃ and —CH₂(CH₂)₈CH₃.

43. The compound of claim 35, wherein A is a substituted alkyl group.

44. The compound of claim 35, wherein A is an alkyl group substituted with an alicyclic group.

45. The compound of claim 35, wherein A is an alkyl group substituted with an alicyclic group selected from the group consisting of. 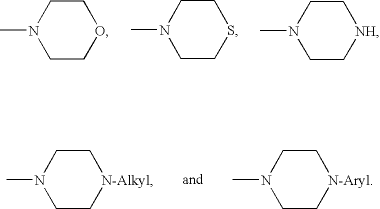
.

46. The compound of claim 35, wherein A is a C₃₋₂₀ alkene.

47. (canceled)

48. (canceled)

49. The compound of claim 35, wherein A is an alkene selected from the group consisting of —CH₂CH═CH₂; —CH₂CH₂CH═CH₂, —CH₂(CH₂)₃CH═CH₂ and —CH₂(CH₂)₅CH═CH₂.

50. The compound of claim 35, wherein A is a substituted or unsubstituted alicyclic group.

51. The compound of claim 35, wherein A is a heterocyclic group.

52. (canceled)

53. The compound of claim 35, wherein A is a heterocyclic ring with 6 ring members.

54. The compound of claim 35, wherein A is a substituted or unsubstituted aryl group, or a substituted or unsubstituted aryl group in which the carbon chain is interrupted by one or more heteroatom(s).

55. The compound of claim 35, wherein A is a substituted or unsubstituted aryl group selected from the group consisting of phenyl, biphenyl, naphthyl, indanyl, and indenyl.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A compound selected from the group consisting of:
N-propyl-7-Oxabicyclo(2.2.1)heptane-2,3-dicarboximide
N-butyl-7-Oxabicyclo(2.2.1)heptane-2,3-dicarboximide;
N-octyl-7-Oxabicyclo(2.2.1)heptane-2,3-dicarboximide;
N-decyl-7-Oxabicyclo(2.2.1)heptane-2,3-dicarboximide;
N-(3-butenyl)-7-Oxabicyclo(2.2.1)heptane-2,3-dicarboximide;
3-(butylcarbamoyl)-7-Oxabicyclo(2.2.1)heptane-2-carboxylic acid;
3-(decylcarbamoyl)-7-Oxabicyclo(2.2.1)heptane-2-carboxylic acid;
3-(4-benzylcarbamoyl)-7-Oxabicyclo(2.2.1)heptane-2-carboxylic acid;
3-(Ethylmorpholine carbamoyl)-7-Oxabicyclo(2.2.1)heptane-2-carboxylic acid;
N-Butanoic acid-7-Oxabicyclo(2.2.1)heptane-2,3-dicarboximide;
N-Hexanoic acid-7-Oxabicyclo(2.2.1)heptane-2,3-dicarboximide;
N-Ethyl alcohol-7-Oxabicyclo(2.2.1)heptane-2,3-dicarboximide;
N-2-Methoxypropyl-1-ol-7-Oxabicyclo(2.2.1)heptane-2,3-dicarboximide;
4-Ethyl-7-Oxabicyclo(2.2.1)heptane-2,3-dicarboximide;
4-Sec-butyl-7-Oxabicyclo(2.2.1)heptane-2,3-dicarboximide;
4-Hexyl-7-Oxabicyclo(2.2.1)heptane-2,3-dicarboximide;
4-Cyclohexyl-7-Oxabicyclo(2.2.1)heptane-2,3-dicarboximide;
4-Dodecyl-7-Oxabicyclo(2.2.1)heptane-2,3-dicarboximide;
4-Tetradecyl-7-Oxabicyclo(2.2.1)heptane-2,3-dicarboximide;
4-Octadecyl-7-Oxabicyclo(2.2.1)heptane-2,3-dicarboximide;
4-Allyl-7-Oxabicyclo(2.2.1)heptane-2,3-dicarboximide;
4-Oxiranylmethyl-7-Oxabicyclo(2.2.1)heptane-2,3-dicarboximide;
4-(3-Hydroxypropyl)-7-Oxabicyclo(2.2.1)heptane-2,3-dicarboximide;
4-(6-Hydroxyhexyl)-7-Oxabicyclo(2.2.1)heptane-2,3-dicarboximide;
4-(2-Hydroxy-1-methylethyl)-7-Oxabicyclo(2.2.1)heptane-2,3-dicarboximide;
4-(2-Hydroxy-1,1-dimethylethyl)-7-Oxabicyclo(2.2.1)heptane-2,3-dicarboximide;
4-(1-Hydroxymethylpropyl)-7-Oxabicyclo(2.2.1)heptane-2,3-dicarboximide;
4-(3,5-Dioxo-10-oxa-4-azatricyclo[5.2.1]dec-4-yl)-butyric acid;
6-(3,5-Dioxo-10-oxa-4-azatricyclo[5.2.1]dec-4-yl)-hexanoic acid;
4-(2,3-Dihydroxypropyl)-10-oxa-4-azatricyclo[5.2.1]decane-3,5-dione;
4-(3-Hydroxy-2-methoxypropyl)-10-oxa-4-azatricyclo[5.2.1]decane-3,5-dione;
4-Morpholin-4-yl-7-Oxabicyclo(2.2.1)heptane-2,3-dicarboximide;
4-(2-Morpholin-4-ylethyl)-7-Oxabicyclo(2.2.1)heptane-2,3-dicarboximide dione;
4-(3-Morpholin-4-ylpropyl)-7-Oxabicyclo(2.2.1)heptane-2,3-dicarboximide;
4-Phenyl-7-Oxabicyclo(2.2.1)heptane-2,3-dicarboximide;
4-(4-Hydroxyphenyl)-10-oxa-4-azatricyclo[5.2.1]decane-3,5-dione;
4-(4-Nitrophenyl)-7-Oxabicyclo(2.2.1)heptane-2,3-dicarboximide;
4-(3,5-Dioxo-10-oxa-4-azatricyclo[5.2.1]dec-4-yl)benzoic acid;
4-Benzyl-10-oxa-4-azatricyclo[5.2.1]decane-3,5-dione;
4-(4-Methoxybenzyl)-7-Oxabicyclo(2.2.1)heptane-2,3-dicarboximide;
4-(3,4-Dimethoxybenzyl)-7-Oxabicyclo(2.2.1)heptane-2,3-dicarboximide;
4-(2-Aminobenzyl)-7-Oxabicyclo(2.2.1)heptane-2,3-dicarboximide;
Bis-3,6-epoxycyclohexane-1,2-dicarboximido)-trimethylene (Bis-norcantharimide-propyl linker);
Bis-3,6-epoxycyclohexane-1,2-dicarboximido)-dodecylmethyle (Bis-norcantharimide-dodecyl linker);
a salt thereof, and a solvate thereof.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. A method of controlling an invertebrate pest, comprising contacting said pest with an effective amount of N-octyl-7-Oxabicyclo(2.2.1)heptane-2,3-dicarboximide, N-decyl-7-Oxabicyclo(2.2.1)heptane-2,3-dicarboximide and combinations thereof.

106. A compound selected from the group consisting of N-octyl-7-Oxabicyclo(2.2.1)heptane-2,3-dicarboximide and N-decyl-7-Oxabicyclo(2.2.1)heptane-2,3-dicarboximide


SEQUENCE LISTING (21 sequences)

SEQ ID NO: 1 - protein, 316 aa, Trichostrongylus vitrinus
MDTTQLITNLLSVGLPDKGLTKTVSENDIMEVLGKAREMFLSQPAMVELDSPVKICGDTHGQYPDLLRLFNKGGFPPLSNYLFLGDYVDRGKQNLEVILLVIAYKLKFPKNFFLLRGNHECANVNRAYGFYDECMRRYQSQRMWQLFQDVFCVMPLTALVGEKILCMHGGLFPHLESFDQLRNIPRPTEAIGATLEMDLLWADPVIGLNGFQANMRGASYGFGPDVLAKYCQTLNIDLVARAHQVVQDGYEFFGGRKLVTIFSAPHYCGQFDNAAAMMTVDENLQCSFEIFRPSVGKPQPKIIPTTVGSPAAPPCQ

SEQ ID NO: 2 - DNA, 951 bp, Trichostrongylus vitrinus
atggacactactcaattgatcactaaccttcttagcgtgggactcccagataaaggacttacaaaaactgtttctgaaaatgatattatggaagtactaggtaaagcacgcgaaatgtttctttctcagccagctatggttgagcttgactcacctgttaaaatatgtggagacactcatggacagtatcctgatttgctaaggcttttcaataagggtggatttcctccattatcgaattacttatttctgggtgattatgtagatagaggaaaacaaaatcttgaagtaatactacttgttattgcctacaagctgaaatttcccaaaaatttctttctgttgcgaggaaatcacgagtgtgctaatgtgaatcgtgcctacggattctatgatgaatgcatgcgaagatatcaaagccaaagaatgtggcagcttttccaggacgttttttgcgtgatgcctttgacagcacttgttggagaaaaaattttgtgcatgcatggaggactatttcctcacttagagtcttttgatcagctcagaaatattcccaggcctaccgaagcgattggagcaaccctggaaatggatttgctttgggctgatccagtaataggattgaatggttttcaggcaaacatgcgaggtgcctcgtatggttttggacccgatgttttggcaaagtactgtcaaacgctcaatattgatctggttgcacgagctcaccaagttgtacaagatggctacgaattttttggaggtcgaaagcttgtgacgattttctcggctcctcactattgtggtcaattcgataatgctgccgcgatgatgaccgttgatgaaaatttgcagtgttcgttcgagatttttcgcccttccgttggaaaacctcaaccgaaaattattcccactactgtagggtctccagcagcgcctccttgtcaatga

SEQ ID NO: 3 - DNA, 1062 bp, Trichostrongylus vitrinus
gccattacggccagggattttcattatttttatgttttaagtgtggtattacgacagacaaataccaaatacgaaatcatggacactactcaattgatcactaaccttcttagcgtgggactcccagataaaggacttacaaaaactgtttctgaaaatgatattatggaagtactaggtaaagcacgcgaaatgtttctttctcagccagctatggttgagcttgactcacctgttaaaatatgtggagacactcatggacagtatcctgatttgctaaggcttttcaataagggtggatttcctccattatcgaattacttatttctgggtgattatgtagatagaggaaaacaaaatcttgaagtaatactacttgttattgcctacaagctgaaatttcccaaaaatttctttctgttgcgaggaaatcacgagtgtgctaatgtgaatcgtgcctacggattctatgatgaatgcatgcgaagatatcaaagccaaagaatgtggcagcttttccaggacgttttttgcgtgatgcctttgacagcacttgttggagaaaaaattttgtgcatgcatggaggactatttcctcacttagagtcttttgatcagctcagaaatattcccaggcctaccgaagcgattggagcaaccctggaaatggatttgctttgggctgatccagtaataggattgaatggttttcaggcaaacatgcgaggtgcctcgtatggttttggacccgatgttttggcaaagtactgtcaaacgctcaatattgatctggttgcacgagctcaccaagttgtacaagatggctacgaattttttggaggtcgaaagcttgtgacgattttctcggctcctcactattgtggtcaattcgataatgctgccgcgatgatgaccgttgatgaaaatttgcagtgttcgttcgagatttttcgcccttccgttggaaaacctcaaccgaaaattattcccactactgtagggtctccagcagcgcctccttgtcaatgatactcacttactcgataaatttgtttgctccat

SEQ ID NO: 4 - protein, 316 aa, Caenorhabditis elegans
MQNNVVDSIIIDVLSASTHEKPLCKVITEERVLKLLDLALGVFKAQKPMVEVNAPIKVCGDIHGQFPDLLRLFHRGGWPPTANYLFLGDYVDRGRFSIETIVLLLAYKVKFPCNFFLLRGNHECEFVNKTYGFYEECQKRYQSVRMYAAFQDVFNWLPLTGLIATKILCMHGGLSPLMTKEFTLDTLRKIERPTEGKEGLVADLLWADPISGLSGFMNNQRGAGCGFGRDSVLNLCSEFQLDLVCRAHQVVQDGYEFFAGRKLVTIFSAPHYCGQFDNCAAFMSCDEKLQCSFEILRPTTGRLEIREKPLLKDETN

SEQ ID NO: 5 - protein, 333 aa, Caenorhabditis elegans
MASGYRSLDGMHRLLTMISFFLLLENFAARLDIDELILKVLSVGHPEKSLTKTVKEHELIQLCQMAKKSFLDQPVMLEVDAPIKVCGDVHGQYSDVIRMFSIARFPPHSNYIFLGDYVDRGRQNLELITLFLCYKIKFYDRFYMLRGNHECPAVNRVYGFYEECNKRYASTRLWLAFQEAFAAMPFTGLISGRILCMHGGLSPKLTNLDVLRDLTRPMDPPSPSLHIDLLWSDPDNSVIDWLPNVRGVSYIFGPNVVKKQIETLGIDLIARAHQVVQDGYEFFAEKKLVTIFSAPHYCGQFDNSAAIMNVDDNLICSFHVLRPSNRKAKQPAN

SEQ ID NO: 6 - protein, 305 aa, Caenorhabditis elegans
MTATIDVDNLMSRLLNVGMSGGRLTTSVNEQELQTCCAVAKSVFASQASLLEVEPPIIVCGDIHGQYSDLLRIFDKNGFPPDINFLFLGDYVDRGRQNIETICLMFCFKIKYPENFFMLRGNHECPAINRVYGFYEECNRRYKSTRLWSIFQDTFNWMPLCGLIGSRILCMHGGLSPHLQTLDQLRQLPRPQDPPNPSIGIDLLWADPDQWVKGWQANTRGVSYVFGQDVVADVCSRLDIDLVARAHQVVQDGYEFFASKKMVTIFSAPHYCGQFDNSAATMKVDENMVCTFVMYKPTPKSLRKG

SEQ ID NO: 7 - protein, 305 aa, Caenorhabditis elegans
MTAPMDVDNLMSRLLNVGMSGGRLTTSVNEQELQTCCAVAKSVFASQASLLEVEPPIIVCGDIHGQYSDLLRIFDKNGFPPDVNFLFLGDYVDRGRQNIETICLMLCFKIKYPENFFMLRGNHECPAINRVYGFYEECNRRYKSTRLWSIFQDTFNWMPLCGLIGSRILCMHGGLSPHLQTLDQLRQLPRPQDPPNPSIGIDLLWADPDQWVKGWQANTRGVSYVFGQDVVADVCSRLDIDLVARAHQVVQDGYEFFASKKMVTIFSAPHYCGQFDNSAATMKVDENMVCTFVMYKPTPKSMRRG

SEQ ID NO: 8 - protein, 333 aa, Caenorhabditis elegans
MDVEKLNLDNIISRLLEVRGSKPGKNVQLTESEIKGLCQKSREIFLSQPILLELEAPLKICGDVHGQYYDLLRLFEYGGFPPESNYLFLGDYVDRGKQSLETICLLLAYKIKYPENFFLLRGNHECASINRIYGFYDECKRRYNIKLWKTFTDCFNCLPVAAIIDEKIFCCHGGLSPDLQSMEQIRRIMRPTDVPDQGLLCDLLWSDPDKDVTGWGENDRGVSFTFGPEVVAKFLHKHDLDLICRAHQVVEDGYEFFAKRQLVTLFSAPNYCGEFDNAGSMMTVDETLMCSFQILKPADKKKYPYGAGGVGSNRPVTPPRNAPAAQPKKGAKK

SEQ ID NO: 9 - protein, 329 aa, Caenorhabditis elegans
MSNDGDLNIDNLITRLLEVRGCRPGKPVTMSEAEIRALCHKSREIFLSQPILLELEAPLKICGDIHGQYNDLLRLFEYGGFPPEANYLFLGDYVDRGKQSLETICLLLAYKVKYPENFFLLRGNHECASINRIYGFYDECKRRFSIKLWKTFTDCFNCLPIAALIDEKIFCCHGGLSPDLQNMEQIRRVMRPTDVPDTGLLCDLLWSDPDKDVTGWGENDRGVSFTFGPDVVAKFLNRHDLDLICRAHQVVEDGYEFFAKRQLVTLFSAPNYCGEFDNAGGMMSVDETLMCSFQILKPSEKKAKYQYQGMNSGRPAVGGGRPGTTAGKK

SEQ ID NO: 10 - protein, 311 aa, Oesophagostomum dentatum
MAQLDVDSLMSRLLNVGMAGGRLTTSVSEQELQQCCFVARQVFISQSSLIECEPPLVVCGDIHGQYSDLLRIFDKNGFPPETNYLFLGDYVDRGRQNIETICLMFCYRIKYPESFFMLRGNHECPAINRIYGFYEECNRRYHSSRLWSSFQDTFNWMPLCGFIAGRILCMHGGLSPQLTSIDQLRNLPRPQDPPNPSMGIDLLWADPDQWVKGWQANTRGVSYVFGQDVVFETCQKLNIDLIARAHQVVQDGYEFFASKKMVTIFSAPHYCGQFDNFAATMKVSEDLVCNFAMYKPTAKALRMAAGVSRAS

SEQ ID NO: 11 - protein, 303 aa, Trypanosoma brucei
MSVDVDAIIDKLLEVRLSKPGKQVSLSENDVKNLVMRSREILLSQPALLELEAPIKICGDIHGQYYDLIRLFDNGGFPPSANYLFLGDYVDRGKQGLETICLVLAFKVKFPENFFILRGNHECASINRIYGFFDECKRRYNIRLWKVFTDTFNCLPVACIIDDKIFCCHGGLSPDLQSMEQIKKIERPCDVADTGLICDLLWSDPEEGLSGWGENDRGVSYTFGQDIVAKFLSRHDFDLIVRAHQVVEDGYQFFATRQLITIFSAPNYCNEFDNSGAVMSVDADLLCSFQILKPSVKKPKYFQ

SEQ ID NO: 12 - protein, 328 aa, Debaryomyces hansenii
MADHQEADIDSIVDRLLEVRGSRPGKQVTLLEHEIRYLCTKAREIFIQQPILLELEAPIKICGDIHGQYYDLLRLFEYGGFPPEANYLFLGDYVDRGKQSLETICLLLAYKIKYPENFFILRGNHECASINRIYGFYDECKRRYNIKLWKTFTDCFNCLPIAAIIDEKIFTMHGGLSPDLNSMEQIRRVMRPTDIPDVGLLCDLLWSDPDKDITGWSENDRGVSFTFGPDVVSRFLQKHDMDLICRAHQVVEDGYEFFSKRQLVTLFSAPNYCGEFDNAGAMMSVDESLLCSFQILKPADKKPRYPQSNNISNNRPVTPPRKPRRGSK

SEQ ID NO: 13 - protein, 317 aa, Nicotiana tabacum
MAQNNEQQQQGQGLIEAGVLDDIINRLLEFRNARTVRQVQLSEAEIRSLCSASREIFLQQPNLLDLKPPIKICGDIHGQYGDLLRLFEYGGFPPEANYLFLGDYVDRGKQSLETICLLLAYKIKYPENFFLLRGNHECASINRIYGFYDECKRRFNVKLWKCFTECFNCLPVAALIDEKILCMHGGLSPVLTNLDQIRNLPRPTDVPDSGLLCDLLWSDPSREVKGWGMNDRGVSYTFGPDKVAEFLMQHDMDLVCRAHQVVEDGYEFFAERQLVTIFSAPNYCGEFDNAGAMMSVDESLMCSFQILKPTDRKPRFL

SEQ ID NO: 14 - protein, 327 aa, Homo sapiens [sequence omitted]

SEQ ID NO: 15 - DNA, 24 bp, Artificial
  oligonucleotide primer
cgaggtgcctcgtatggttttgga

SEQ ID NO: 16 - DNA, 27 bp, Artificial
  oligonucleotide primer
ggctgagaaagaaacatttcgcgtgct

SEQ ID NO: 17 - DNA, 27 bp, Artificial
  oligonucleotide primer
atggacactactcaattgatcactaac

SEQ ID NO: 18 - DNA, 20 bp, Artificial
  oligonucleotide primer
tcattgacaaggaggcgctg

SEQ ID NO: 19 - DNA, 20 bp, Artificial
  oligonucleotide primer
gcataagctctcgcgttacc

SEQ ID NO: 20 - DNA, 20 bp, Artificial
  oligonucleotide primer
gagagggacagcaggttcac

SEQ ID NO: 21 - RNA, 19 nt, Artificial
  siRNA
uaauuuucgguugagguuu